SEQUENCE LISTING (81 sequences)

SEQ ID NO: 1 - protein, 736 aa, adeno-associated AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, Artificial Sequence
  AAV isolate
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, Artificial Sequence
  AAV isolate
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAVDAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 12 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 13 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 1527 bp, Artificial Sequence
  Synthetic polynucleotide
atgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgcc

SEQ ID NO: 15 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 16 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 18 - DNA, 145 bp, Artificial Sequence
  AAV2 5′ ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 19 - DNA, 145 bp, Artificial Sequence
  AAV2 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 20 - DNA, 167 bp, Artificial Sequence
  AAV5 5′ ITR
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 21 - DNA, 167 bp, Artificial Sequence
  AAV5 3′ ITR
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 22 - protein, 621 aa, Artificial Sequence
  AAV2 Rep
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 23 - protein, 509 aa, Homo sapiens
MSMGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 24 - DNA, 1527 bp, Homo sapiens
atgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcc

SEQ ID NO: 25 - DNA, 278 bp, Artificial Sequence
  Synthetic polynucleotide
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 26 - DNA, 106 bp, Artificial Sequence
  5′ ITR
ctgcgcgctcgctcgctcactgaggccgcccgggcaaagcccgggcgtcgggcgacctttggtcgcccggcctcagtgagcgagcgagcgcgcagagagggagtgg

SEQ ID NO: 27 - DNA, 143 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 28 - DNA, 1873 bp, Artificial Sequence
  Synthetic polynucleotide
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 29 - protein, 374 aa, Homo sapiens
MAAPALGLVCGRCPELGLVLLLLLLSLLCGAAGSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD

SEQ ID NO: 30 - DNA, 2718 bp, Artificial Sequence
  Synthetic polynucleotide
atgagcatgggcgcccccagaagcctgttacttgctttagctgctggccttgcagtggcaaggccccctaacatcgtgctgatctttgcagatgacttgggatatggggatcttggttgttatggccacccatcaagcacaactcccaatctggatcagttggctgcaggaggtctgaggtttacagacttttatgttccagtctccctgtgcactccttctcgggctgccctgcttactgggaggctccctgtgagaatgggtatgtaccctggagtgttggtcccatccagcaggggagggctgcccctggaagaggtgacagtggcagaggtgctggcagcacgaggctatctgactggcatggcaggcaagtggcacctgggtgtagggccagagggtgctttcctgcctccccatcagggctttcataggtttctgggaatcccatactctcatgaccaaggaccctgccagaacctcacctgtttcccccctgcaacaccatgtgatgggggctgtgatcaaggtctggttcctataccactgcttgctaatctttcagtggaagctcaaccaccctggctgcctggcttggaggctagatacatggccttcgcacatgatctgatggcagatgcccagagacaagataggcctttcttcctctactatgcatctcaccacacccactatcctcagttctcaggccaatcatttgctgagcgtagtggcaggggcccatttggggacagtttgatggaactggatgccgcagttggtaccctcatgacagcaataggggacttaggtttgctggaggaaacattggtaattttcacagctgataatggccctgagacaatgagaatgtctaggggaggctgctctggtcttctgaggtgtggtaaagggactacatatgagggaggagtgagggaaccagctcttgccttttggccaggtcacatagcccctggagttacacatgaactagcttcttccctggacttgcttcctacactggcagccctggcaggtgcccctctccctaatgtaactttagatggatttgacctctctccactacttttagggacagggaaaagtccaaggcagtccttattcttctatccttcctacccagatgaggtgaggggtgtttttgccgtgaggactgggaaatacaaagctcatttttttacccagggatcagctcattcagacaccacagctgatcctgcctgtcatgccagcagtagcttgacagcacatgagcctcccttactgtatgacctgagcaaggacccaggggagaactataacctgcttgggggggttgctggggccaccccagaagtgcttcaggcactaaagcagctgcaactgcttaaagcacagttggatgctgcagtgacctttggcccttcccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctatggctgccccagccctggggctggtgtgtggcagatgccctgagctgggcctggtgctgcttctcctgctgctgagcctcctgtgtggtgctgctggctctcaggaagcagggacaggagcaggagcaggttctctggctggctcatgcggttgtgggaccccccagaggccaggggctcatgggtcctctgcagctgcccacaggtactcaagggaagcaaatgcccctggccccgtacctggggaaaggcaacttgctcactccaagatggttcctatccctgcaggagtttttactatgggaactgatgaccctcagatcaagcaggatggtgaagcaccagctaggagagtcacaattgatgccttctatatggatgcctatgaagtgtcaaacacagaatttgagaaatttgtaaacagcactggataccttacagaggctgagaaatttggtgacagttttgtttttgaaggcatgctaagtgagcaggtgaagaccaatatccaacaggcagtggctgcagccccctggtggctgcctgttaaaggagccaattggagacacccagagggaccagactcaactatcctccacaggcctgaccaccctgtgctgcatgtgtcctggaatgatgcagtggcatactgcacctgggctgggaaaaggttaccaacagaggcagaatgggagtattcctgccggggtggactgcacaacagactgttcccctggggcaataagctgcaacctaaaggacagcattatgccaatatttggcagggagagttcccagtcacaaacactggtgaggatggcttccagggaactgcccctgtggatgctttcccacccaatggctatgggttgtacaatatagttgggaatgcctgggagtggacttctgactggtggacggtccatcacagtgtggaagagacactgaacccaaaggggcccccctcaggcaaggacagagtcaagaaaggtggctcttatatgtgtcacagaagctattgctacagatataggtgtgctgcaagaagtcagaacacccctgacagctcagctagcaatctgggatttagatgtgcagcagatagactccccaccatggactga

SEQ ID NO: 31 - DNA, 93 bp, Artificial Sequence
  Synthetic polynucleotide
ctctaaggtaaatataaaatttttaagtgtataatgtgttaaactactgattctaattgtttctctcttttagattccaacctttggaactga

SEQ ID NO: 32 - DNA, 1017 bp, Artificial Sequence
  Synthetic polynucleotide
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 33 - protein, 79 aa, Homo sapiens
GDVCQDCIQMVTDIQTAVRTNSTFVQALVEHVKEECDRLGPGMADICKNYISQYSEIAIQMMMHMQPKEICALVGFCDE

SEQ ID NO: 34 - protein, 8 aa, Artificial Sequence
  Synthetic polynucleotide
  MISC_FEATURE (1)..(1): Xaa is D or G
  MISC_FEATURE (2)..(2): Xaa is V or I
  MISC_FEATURE (4)..(4): Xaa is any amino acid
XXEXNPGP

SEQ ID NO: 35 - DNA, 92 bp, Artificial Sequence
  Synthetic polynucleotide
aagaggtaagggtttaagggatggttggttggtggggtattaatgtttaattacctggagcacctgcctgaaatcactttttttcaggttgg

SEQ ID NO: 36 - DNA, 1676 bp, Artificial Sequence
  Synthetic polynucleotide
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 37 - protein, 16 aa, Artificial Sequence
  T2A peptide
EGRGSLLTCGDVEENP

SEQ ID NO: 38 - protein, 16 aa, Artificial Sequence
  P2A peptide
ATNFSLLKQAGDVEEN

SEQ ID NO: 39 - DNA, 540 bp, Artificial Sequence
  Synthetic polynucleotide
cctgcaggctcaccagtgtttgtgactgggaactctccctgccaaatattggcataatgctgtcctttaggttgcagcttattgccccaggggaacagtctgttgtgcagtccaccccggcaggaatactcccattctgcctctgttggtaaccttttcccagcccaggtgcagtatgccactgcatcattccaggacacatgcagcacagggtggtcaggcctgtggaggatagttgagtctggtccctctgggtgtctccaattggctcctttaacaggcagccaccagggggctgcagccactgcctgttggatattggtcttcacctgctcacttagcatgccttcaaaaacaaaactgtcaccaaatttctcagcctctgtaaggtatccagtgctgtttacaaatttctcaaattctgtgtttgacacttcataggcatccatatagaaggcatcaattgtgactctcctagctggtgcttcaccatcctgcttgatctgagggtcatcagttcccatagtaaaaactcctgca

SEQ ID NO: 40 - DNA, 1168 bp, Artificial Sequence
  Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 41 - DNA, 3416 bp, Artificial Sequence
  Synthetic polynucleotide
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctgagattctagagtcgagccgcggactagtaacttgtttattgcagcttataatggttacaaataaagcaatagcatcacaaatttcacaaataaagcatttttttcactgcattctagttgtggtttgtccaaactcatcaatgtatctta

SEQ ID NO: 42 - DNA, 122 bp, Artificial Sequence
  Synthetic polynucleotide
aacttgtttattgcagcttataatggttacaaataaagcaatagcatcacaaatttcacaaataaagcatttttttcactgcattctagttgtggtttgtccaaactcatcaatgtatctta

SEQ ID NO: 43 - DNA, 133 bp, Artificial Sequence
  Synthetic polynucleotide
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 44 - DNA, 3416 bp, Artificial Sequence
  Synthetic polynucleotide
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgcctaagattctagagtcgagccgcggactagtaacttgtttattgcagcttataatggttacaaataaagcaatagcatcacaaatttcacaaataaagcatttttttcactgcattctagttgtggtttgtccaaactcatcaatgtatctta

SEQ ID NO: 45 - DNA, 198 bp, Artificial Sequence
  Synthetic polynucleotide
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 46 - DNA, 3416 bp, Artificial Sequence; identical to SEQ ID NO: 44
  Synthetic polynucleotide

SEQ ID NO: 47 - DNA, 3949 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctgagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 48 - DNA, 3949 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgcctaagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 49 - DNA, 4500 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcgatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgcctaagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttacctgcaggctcaccagtgtttgtgactgggaactctccctgccaaatattggcataatgctgtcctttaggttgcagcttattgccccaggggaacagtctgttgtgcagtccaccccggcaggaatactcccattctgcctctgttggtaaccttttcccagcccaggtgcagtatgccactgcatcattccaggacacatgcagcacagggtggtcaggcctgtggaggatagttgagtctggtccctctgggtgtctccaattggctcctttaacaggcagccaccagggggctgcagccactgcctgttggatattggtcttcacctgctcacttagcatgccttcaaaaacaaaactgtcaccaaatttctcagcctctgtaaggtatccagtgctgtttacaaatttctcaaattctgtgtttgacacttcataggcatccatatagaaggcatcaattgtgactctcctagctggtgcttcaccatcctgcttgatctgagggtcatcagttcccatagtaaaaactcctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 50 - DNA, 6612 bp, Artificial Sequence
  Synthetic polynucleotide
cgccagggttttcccagtcacgacgttgtaaaacgacggccagtgccaagcttgcatgcctgcatttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctgagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatccccgggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggcgaacttttgctgagttgaaggatcagatcacgcatcttcccgacaacgcagaccgttccgtggcaaagcaaaagttcaaaatcagtaaccgtcagtgccgataagttcaaagttaaacctggtgttgataccaacattgaaacgctgatcgaaaacgcgctgaaaaacgctgctgaatgtgcgagcttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgatccgtcgagaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgcattcgccattcaggctgcgcaactgttgggaagggcgatcggtgcgggcctcttcgctattacgccagctggcgaaagggggatgtgctgcaaggcgattaagttgggtaa

SEQ ID NO: 51 - DNA, 5792 bp, Artificial Sequence
  Synthetic polynucleotide
gtcaggtggcacttttcggggaaatgtggcatgcctgcatttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgcctaagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatccccgggtaccgaggacgaattctctagatatcgctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctgagggcccaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgatatcaagcttgaattcgtt

SEQ ID NO: 52 - DNA, 6342 bp, Artificial Sequence
  Synthetic polynucleotide
tttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgatatcaagcttgaattcgtgtcaggtggcacttttcggggaaatgtggcatgcctgcatttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcgatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgcctaagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttacctgcaggctcaccagtgtttgtgactgggaactctccctgccaaatattggcataatgctgtcctttaggttgcagcttattgccccaggggaacagtctgttgtgcagtccaccccggcaggaatactcccattctgcctctgttggtaaccttttcccagcccaggtgcagtatgccactgcatcattccaggacacatgcagcacagggtggtcaggcctgtggaggatagttgagtctggtccctctgggtgtctccaattggctcctttaacaggcagccaccagggggctgcagccactgcctgttggatattggtcttcacctgctcacttagcatgccttcaaaaacaaaactgtcaccaaatttctcagcctctgtaaggtatccagtgctgtttacaaatttctcaaattctgtgtttgacacttcataggcatccatatagaaggcatcaattgtgactctcctagctggtgcttcaccatcctgcttgatctgagggtcatcagttcccatagtaaaaactcctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatccccgggtaccgaggacgaattctctagatatcgctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctgagggcccaaatgtaatcacctggctcaccttcgggtgggcctttctgcgttgctggcgtt

SEQ ID NO: 53 - DNA, 6612 bp, Artificial Sequence
  Synthetic polynucleotide
cgccagggttttcccagtcacgacgttgtaaaacgacggccagtgccaagcttgcatgcctgcatttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgcctaagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatccccgggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggcgaacttttgctgagttgaaggatcagatcacgcatcttcccgacaacgcagaccgttccgtggcaaagcaaaagttcaaaatcagtaaccgtcagtgccgataagttcaaagttaaacctggtgttgataccaacattgaaacgctgatcgaaaacgcgctgaaaaacgctgctgaatgtgcgagcttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgatccgtcgagaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgcattcgccattcaggctgcgcaactgttgggaagggcgatcggtgcgggcctcttcgctattacgccagctggcgaaagggggatgtgctgcaaggcgattaagttgggtaa

SEQ ID NO: 54 - DNA, 918 bp, Artificial Sequence
  Synthetic polynucleotide
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccaagag

SEQ ID NO: 55 - DNA, 953 bp, Artificial Sequence
  Synthetic polynucleotide
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 56 - DNA, 37 bp, Artificial Sequence
  Synthetic polynucleotide
gtagataagtagcatggcgggttaatcattaactaca

SEQ ID NO: 57 - DNA, 180 bp, Artificial Sequence
  3′ ITR
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 58 - DNA, 380 bp, Artificial Sequence
  Synthetic polynucleotide
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 59 - DNA, 1246 bp, Artificial Sequence
  Synthetic polynucleotide
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 60 - DNA, 95 bp, Artificial Sequence
  Synthetic polynucleotide
cctctgctaaccatgttcatgccttcttctttttcctacagctcctgggcaacgtgctggttattgtgctgtctcatcattttggcaaagaattc

SEQ ID NO: 61 - DNA, 1061 bp, Artificial Sequence
  Synthetic polynucleotide
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 62 - DNA, 1527 bp, Artificial Sequence
  Synthetic polynucleotide
atgagcatgggcgcccccagaagcctgttacttgctttagctgctggccttgcagtggcaaggccccctaacatcgtgctgatctttgcagatgacttgggatatggggatcttggttgttatggccacccatcaagcacaactcccaatctggatcagttggctgcaggaggtctgaggtttacagacttttatgttccagtctccctgtgcactccttctcgggctgccctgcttactgggaggctccctgtgagaatgggtatgtaccctggagtgttggtcccatccagcaggggagggctgcccctggaagaggtgacagtggcagaggtgctggcagcacgaggctatctgactggcatggcaggcaagtggcacctgggtgtagggccagagggtgctttcctgcctccccatcagggctttcataggtttctgggaatcccatactctcatgaccaaggaccctgccagaacctcacctgtttcccccctgcaacaccatgtgatgggggctgtgatcaaggtctggttcctataccactgcttgctaatctttcagtggaagctcaaccaccctggctgcctggcttggaggctagatacatggccttcgcacatgatctgatggcagatgcccagagacaagataggcctttcttcctctactatgcatctcaccacacccactatcctcagttctcaggccaatcatttgctgagcgtagtggcaggggcccatttggggacagtttgatggaactggatgccgcagttggtaccctcatgacagcaataggggacttaggtttgctggaggaaacattggtaattttcacagctgataatggccctgagacaatgagaatgtctaggggaggctgctctggtcttctgaggtgtggtaaagggactacatatgagggaggagtgagggaaccagctcttgccttttggccaggtcacatagcccctggagttacacatgaactagcttcttccctggacttgcttcctacactggcagccctggcaggtgcccctctccctaatgtaactttagatggatttgacctctctccactacttttagggacagggaaaagtccaaggcagtccttattcttctatccttcctacccagatgaggtgaggggtgtttttgccgtgaggactgggaaatacaaagctcatttttttacccagggatcagctcattcagacaccacagctgatcctgcctgtcatgccagcagtagcttgacagcacatgagcctcccttactgtatgacctgagcaaggacccaggggagaactataacctgcttgggggggttgctggggccaccccagaagtgcttcaggcactaaagcagctgcaactgcttaaagcacagttggatgctgcagtgacctttggcccttcccaggtggccagaggcgaggatcccgccctgcagatctgctgccacccaggctgcacacccagacctgcctgctgtcactgccccgacccacacgcc

SEQ ID NO: 63 - DNA, 57 bp, Artificial Sequence
  Synthetic polynucleotide
gctactaacttcagcctgctgaagcaggctggagacgtggaggagaaccctggacct

SEQ ID NO: 64 - DNA, 1122 bp, Homo sapiens
atggctgccccagccctggggctggtgtgtggcagatgccctgagctgggcctggtgctgcttctcctgctgctgagcctcctgtgtggtgctgctggctctcaggaagcagggacaggagcaggagcaggttctctggctggctcatgcggttgtgggaccccccagaggccaggggctcatgggtcctctgcagctgcccacaggtactcaagggaagcaaatgcccctggccccgtacctggggaaaggcaacttgctcactccaagatggttcctatccctgcaggagtttttactatgggaactgatgaccctcagatcaagcaggatggtgaagcaccagctaggagagtcacaattgatgccttctatatggatgcctatgaagtgtcaaacacagaatttgagaaatttgtaaacagcactggataccttacagaggctgagaaatttggtgacagttttgtttttgaaggcatgctaagtgagcaggtgaagaccaatatccaacaggcagtggctgcagccccctggtggctgcctgttaaaggagccaattggagacacccagagggaccagactcaactatcctccacaggcctgaccaccctgtgctgcatgtgtcctggaatgatgcagtggcatactgcacctgggctgggaaaaggttaccaacagaggcagaatgggagtattcctgccggggtggactgcacaacagactgttcccctggggcaataagctgcaacctaaaggacagcattatgccaatatttggcagggagagttcccagtcacaaacactggtgaggatggcttccagggaactgcccctgtggatgctttcccacccaatggctatgggttgtacaatatagttgggaatgcctgggagtggacttctgactggtggacggtccatcacagtgtggaagagacactgaacccaaaggggcccccctcaggcaaggacagagtcaagaaaggtggctcttatatgtgtcacagaagctattgctacagatataggtgtgctgcaagaagtcagaacacccctgacagctcagctagcaatctgggatttagatgtgcagcagatagactccccaccatggac

SEQ ID NO: 65 - DNA, 3739 bp, Artificial Sequence
  Synthetic polynucleotide
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccaagaggtaagggtttaagggatggttggttggtggggtattaatgtttaattacctggagcacctgcctgaaatcactttttttcaggttgggccacccgccgccaccatgagcatgggcgcccccagaagcctgttacttgctttagctgctggccttgcagtggcaaggccccctaacatcgtgctgatctttgcagatgacttgggatatggggatcttggttgttatggccacccatcaagcacaactcccaatctggatcagttggctgcaggaggtctgaggtttacagacttttatgttccagtctccctgtgcactccttctcgggctgccctgcttactgggaggctccctgtgagaatgggtatgtaccctggagtgttggtcccatccagcaggggagggctgcccctggaagaggtgacagtggcagaggtgctggcagcacgaggctatctgactggcatggcaggcaagtggcacctgggtgtagggccagagggtgctttcctgcctccccatcagggctttcataggtttctgggaatcccatactctcatgaccaaggaccctgccagaacctcacctgtttcccccctgcaacaccatgtgatgggggctgtgatcaaggtctggttcctataccactgcttgctaatctttcagtggaagctcaaccaccctggctgcctggcttggaggctagatacatggccttcgcacatgatctgatggcagatgcccagagacaagataggcctttcttcctctactatgcatctcaccacacccactatcctcagttctcaggccaatcatttgctgagcgtagtggcaggggcccatttggggacagtttgatggaactggatgccgcagttggtaccctcatgacagcaataggggacttaggtttgctggaggaaacattggtaattttcacagctgataatggccctgagacaatgagaatgtctaggggaggctgctctggtcttctgaggtgtggtaaagggactacatatgagggaggagtgagggaaccagctcttgccttttggccaggtcacatagcccctggagttacacatgaactagcttcttccctggacttgcttcctacactggcagccctggcaggtgcccctctccctaatgtaactttagatggatttgacctctctccactacttttagggacagggaaaagtccaaggcagtccttattcttctatccttcctacccagatgaggtgaggggtgtttttgccgtgaggactgggaaatacaaagctcatttttttacccagggatcagctcattcagacaccacagctgatcctgcctgtcatgccagcagtagcttgacagcacatgagcctcccttactgtatgacctgagcaaggacccaggggagaactataacctgcttgggggggttgctggggccaccccagaagtgcttcaggcactaaagcagctgcaactgcttaaagcacagttggatgctgcagtgacctttggcccttcccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctatggctgccccagccctggggctggtgtgtggcagatgccctgagctgggcctggtgctgcttctcctgctgctgagcctcctgtgtggtgctgctggctctcaggaagcagggacaggagcaggagcaggttctctggctggctcatgcggttgtgggaccccccagaggccaggggctcatgggtcctctgcagctgcccacaggtactcaagggaagcaaatgcccctggccccgtacctggggaaaggcaacttgctcactccaagatggttcctatccctgcaggagtttttactatgggaactgatgaccctcagatcaagcaggatggtgaagcaccagctaggagagtcacaattgatgccttctatatggatgcctatgaagtgtcaaacacagaatttgagaaatttgtaaacagcactggataccttacagaggctgagaaatttggtgacagttttgtttttgaaggcatgctaagtgagcaggtgaagaccaatatccaacaggcagtggctgcagccccctggtggctgcctgttaaaggagccaattggagacacccagagggaccagactcaactatcctccacaggcctgaccaccctgtgctgcatgtgtcctggaatgatgcagtggcatactgcacctgggctgggaaaaggttaccaacagaggcagaatgggagtattcctgccggggtggactgcacaacagactgttcccctggggcaataagctgcaacctaaaggacagcattatgccaatatttggcagggagagttcccagtcacaaacactggtgaggatggcttccagggaactgcccctgtggatgctttcccacccaatggctatgggttgtacaatatagttgggaatgcctgggagtggacttctgactggtggacggtccatcacagtgtggaagagacactgaacccaaaggggcccccctcaggcaaggacagagtcaagaaaggtggctcttatatgtgtcacagaagctattgctacagatataggtgtgctgcaagaagtcagaacacccctgacagctcagctagcaatctgggatttagatgtgcagcagatagactccccaccatggactga

SEQ ID NO: 66 - DNA, 54 bp, Artificial Sequence
  Synthetic polynucleotide
gagggcagaggaagtcttctaacatgcggtgacgtggaggagaatcccggccct

SEQ ID NO: 67 - DNA, 3686 bp, Artificial Sequence
  Synthetic polynucleotide
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaaggctagcgccgccaccatgagcatgggcgcccccagaagcctgttacttgctttagctgctggccttgcagtggcaaggccccctaacatcgtgctgatctttgcagatgacttgggatatggggatcttggttgttatggccacccatcaagcacaactcccaatctggatcagttggctgcaggaggtctgaggtttacagacttttatgttccagtctccctgtgcactccttctcgggctgccctgcttactgggaggctccctgtgagaatgggtatgtaccctggagtgttggtcccatccagcaggggagggctgcccctggaagaggtgacagtggcagaggtgctggcagcacgaggctatctgactggcatggcaggcaagtggcacctgggtgtagggccagagggtgctttcctgcctccccatcagggctttcataggtttctgggaatcccatactctcatgaccaaggaccctgccagaacctcacctgtttcccccctgcaacaccatgtgatgggggctgtgatcaaggtctggttcctataccactgcttgctaatctttcagtggaagctcaaccaccctggctgcctggcttggaggctagatacatggccttcgcacatgatctgatggcagatgcccagagacaagataggcctttcttcctctactatgcatctcaccacacccactatcctcagttctcaggccaatcatttgctgagcgtagtggcaggggcccatttggggacagtttgatggaactggatgccgcagttggtaccctcatgacagcaataggggacttaggtttgctggaggaaacattggtaattttcacagctgataatggccctgagacaatgagaatgtctaggggaggctgctctggtcttctgaggtgtggtaaagggactacatatgagggaggagtgagggaaccagctcttgccttttggccaggtcacatagcccctggagttacacatgaactagcttcttccctggacttgcttcctacactggcagccctggcaggtgcccctctccctaatgtaactttagatggatttgacctctctccactacttttagggacagggaaaagtccaaggcagtccttattcttctatccttcctacccagatgaggtgaggggtgtttttgccgtgaggactgggaaatacaaagctcatttttttacccagggatcagctcattcagacaccacagctgatcctgcctgtcatgccagcagtagcttgacagcacatgagcctcccttactgtatgacctgagcaaggacccaggggagaactataacctgcttgggggggttgctggggccaccccagaagtgcttcaggcactaaagcagctgcaactgcttaaagcacagttggatgctgcagtgacctttggcccttcccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctatggctgccccagccctggggctggtgtgtggcagatgccctgagctgggcctggtgctgcttctcctgctgctgagcctcctgtgtggtgctgctggctctcaggaagcagggacaggagcaggagcaggttctctggctggctcatgcggttgtgggaccccccagaggccaggggctcatgggtcctctgcagctgcccacaggtactcaagggaagcaaatgcccctggccccgtacctggggaaaggcaacttgctcactccaagatggttcctatccctgcaggagtttttactatgggaactgatgaccctcagatcaagcaggatggtgaagcaccagctaggagagtcacaattgatgccttctatatggatgcctatgaagtgtcaaacacagaatttgagaaatttgtaaacagcactggataccttacagaggctgagaaatttggtgacagttttgtttttgaaggcatgctaagtgagcaggtgaagaccaatatccaacaggcagtggctgcagccccctggtggctgcctgttaaaggagccaattggagacacccagagggaccagactcaactatcctccacaggcctgaccaccctgtgctgcatgtgtcctggaatgatgcagtggcatactgcacctgggctgggaaaaggttaccaacagaggcagaatgggagtattcctgccggggtggactgcacaacagactgttcccctggggcaataagctgcaacctaaaggacagcattatgccaatatttggcagggagagttcccagtcacaaacactggtgaggatggcttccagggaactgcccctgtggatgctttcccacccaatggctatgggttgtacaatatagttgggaatgcctgggagtggacttctgactggtggacggtccatcacagtgtggaagagacactgaacccaaaggggcccccctcaggcaaggacagagtcaagaaaggtggctcttatatgtgtcacagaagctattgctacagatataggtgtgctgcaagaagtcagaacacccctgacagctcagctagcaatctgggatttagatgtgcagcagatagactccccaccatggactga

SEQ ID NO: 68 - DNA, 4346 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccaagaggtaagggtttaagggatggttggttggtggggtattaatgtttaattacctggagcacctgcctgaaatcactttttttcaggttgggccacccgccgccaccatgagcatgggcgcccccagaagcctgttacttgctttagctgctggccttgcagtggcaaggccccctaacatcgtgctgatctttgcagatgacttgggatatggggatcttggttgttatggccacccatcaagcacaactcccaatctggatcagttggctgcaggaggtctgaggtttacagacttttatgttccagtctccctgtgcactccttctcgggctgccctgcttactgggaggctccctgtgagaatgggtatgtaccctggagtgttggtcccatccagcaggggagggctgcccctggaagaggtgacagtggcagaggtgctggcagcacgaggctatctgactggcatggcaggcaagtggcacctgggtgtagggccagagggtgctttcctgcctccccatcagggctttcataggtttctgggaatcccatactctcatgaccaaggaccctgccagaacctcacctgtttcccccctgcaacaccatgtgatgggggctgtgatcaaggtctggttcctataccactgcttgctaatctttcagtggaagctcaaccaccctggctgcctggcttggaggctagatacatggccttcgcacatgatctgatggcagatgcccagagacaagataggcctttcttcctctactatgcatctcaccacacccactatcctcagttctcaggccaatcatttgctgagcgtagtggcaggggcccatttggggacagtttgatggaactggatgccgcagttggtaccctcatgacagcaataggggacttaggtttgctggaggaaacattggtaattttcacagctgataatggccctgagacaatgagaatgtctaggggaggctgctctggtcttctgaggtgtggtaaagggactacatatgagggaggagtgagggaaccagctcttgccttttggccaggtcacatagcccctggagttacacatgaactagcttcttccctggacttgcttcctacactggcagccctggcaggtgcccctctccctaatgtaactttagatggatttgacctctctccactacttttagggacagggaaaagtccaaggcagtccttattcttctatccttcctacccagatgaggtgaggggtgtttttgccgtgaggactgggaaatacaaagctcatttttttacccagggatcagctcattcagacaccacagctgatcctgcctgtcatgccagcagtagcttgacagcacatgagcctcccttactgtatgacctgagcaaggacccaggggagaactataacctgcttgggggggttgctggggccaccccagaagtgcttcaggcactaaagcagctgcaactgcttaaagcacagttggatgctgcagtgacctttggcccttcccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctatggctgccccagccctggggctggtgtgtggcagatgccctgagctgggcctggtgctgcttctcctgctgctgagcctcctgtgtggtgctgctggctctcaggaagcagggacaggagcaggagcaggttctctggctggctcatgcggttgtgggaccccccagaggccaggggctcatgggtcctctgcagctgcccacaggtactcaagggaagcaaatgcccctggccccgtacctggggaaaggcaacttgctcactccaagatggttcctatccctgcaggagtttttactatgggaactgatgaccctcagatcaagcaggatggtgaagcaccagctaggagagtcacaattgatgccttctatatggatgcctatgaagtgtcaaacacagaatttgagaaatttgtaaacagcactggataccttacagaggctgagaaatttggtgacagttttgtttttgaaggcatgctaagtgagcaggtgaagaccaatatccaacaggcagtggctgcagccccctggtggctgcctgttaaaggagccaattggagacacccagagggaccagactcaactatcctccacaggcctgaccaccctgtgctgcatgtgtcctggaatgatgcagtggcatactgcacctgggctgggaaaaggttaccaacagaggcagaatgggagtattcctgccggggtggactgcacaacagactgttcccctggggcaataagctgcaacctaaaggacagcattatgccaatatttggcagggagagttcccagtcacaaacactggtgaggatggcttccagggaactgcccctgtggatgctttcccacccaatggctatgggttgtacaatatagttgggaatgcctgggagtggacttctgactggtggacggtccatcacagtgtggaagagacactgaacccaaaggggcccccctcaggcaaggacagagtcaagaaaggtggctcttatatgtgtcacagaagctattgctacagatataggtgtgctgcaagaagtcagaacacccctgacagctcagctagcaatctgggatttagatgtgcagcagatagactccccaccatggactgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 69 - DNA, 4492 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcacctaggtcattctggcctccccctccctcaaggccagtcattctggcctgtccttccccgaaggccagtcattctggcctccccctcccccaaggccagtcattctggccttcccctcccttaaggccagagtactatcgattcacacaaaaaaccaacacactattgcaatgaaaataaatttcctttattaagcttaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaaggctagcgccgccaccatgagcatgggcgcccccagaagcctgttacttgctttagctgctggccttgcagtggcaaggccccctaacatcgtgctgatctttgcagatgacttgggatatggggatcttggttgttatggccacccatcaagcacaactcccaatctggatcagttggctgcaggaggtctgaggtttacagacttttatgttccagtctccctgtgcactccttctcgggctgccctgcttactgggaggctccctgtgagaatgggtatgtaccctggagtgttggtcccatccagcaggggagggctgcccctggaagaggtgacagtggcagaggtgctggcagcacgaggctatctgactggcatggcaggcaagtggcacctgggtgtagggccagagggtgctttcctgcctccccatcagggctttcataggtttctgggaatcccatactctcatgaccaaggaccctgccagaacctcacctgtttcccccctgcaacaccatgtgatgggggctgtgatcaaggtctggttcctataccactgcttgctaatctttcagtggaagctcaaccaccctggctgcctggcttggaggctagatacatggccttcgcacatgatctgatggcagatgcccagagacaagataggcctttcttcctctactatgcatctcaccacacccactatcctcagttctcaggccaatcatttgctgagcgtagtggcaggggcccatttggggacagtttgatggaactggatgccgcagttggtaccctcatgacagcaataggggacttaggtttgctggaggaaacattggtaattttcacagctgataatggccctgagacaatgagaatgtctaggggaggctgctctggtcttctgaggtgtggtaaagggactacatatgagggaggagtgagggaaccagctcttgccttttggccaggtcacatagcccctggagttacacatgaactagcttcttccctggacttgcttcctacactggcagccctggcaggtgcccctctccctaatgtaactttagatggatttgacctctctccactacttttagggacagggaaaagtccaaggcagtccttattcttctatccttcctacccagatgaggtgaggggtgtttttgccgtgaggactgggaaatacaaagctcatttttttacccagggatcagctcattcagacaccacagctgatcctgcctgtcatgccagcagtagcttgacagcacatgagcctcccttactgtatgacctgagcaaggacccaggggagaactataacctgcttgggggggttgctggggccaccccagaagtgcttcaggcactaaagcagctgcaactgcttaaagcacagttggatgctgcagtgacctttggcccttcccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctatggctgccccagccctggggctggtgtgtggcagatgccctgagctgggcctggtgctgcttctcctgctgctgagcctcctgtgtggtgctgctggctctcaggaagcagggacaggagcaggagcaggttctctggctggctcatgcggttgtgggaccccccagaggccaggggctcatgggtcctctgcagctgcccacaggtactcaagggaagcaaatgcccctggccccgtacctggggaaaggcaacttgctcactccaagatggttcctatccctgcaggagtttttactatgggaactgatgaccctcagatcaagcaggatggtgaagcaccagctaggagagtcacaattgatgccttctatatggatgcctatgaagtgtcaaacacagaatttgagaaatttgtaaacagcactggataccttacagaggctgagaaatttggtgacagttttgtttttgaaggcatgctaagtgagcaggtgaagaccaatatccaacaggcagtggctgcagccccctggtggctgcctgttaaaggagccaattggagacacccagagggaccagactcaactatcctccacaggcctgaccaccctgtgctgcatgtgtcctggaatgatgcagtggcatactgcacctgggctgggaaaaggttaccaacagaggcagaatgggagtattcctgccggggtggactgcacaacagactgttcccctggggcaataagctgcaacctaaaggacagcattatgccaatatttggcagggagagttcccagtcacaaacactggtgaggatggcttccagggaactgcccctgtggatgctttcccacccaatggctatgggttgtacaatatagttgggaatgcctgggagtggacttctgactggtggacggtccatcacagtgtggaagagacactgaacccaaaggggcccccctcaggcaaggacagagtcaagaaaggtggctcttatatgtgtcacagaagctattgctacagatataggtgtgctgcaagaagtcagaacacccctgacagctcagctagcaatctgggatttagatgtgcagcagatagactccccaccatggactgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 70 - DNA, 7537 bp, Artificial Sequence
  Synthetic polynucleotide
aaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttagatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtggcatgcctgcatttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccaagaggtaagggtttaagggatggttggttggtggggtattaatgtttaattacctggagcacctgcctgaaatcactttttttcaggttgggccacccgccgccaccatgagcatgggcgcccccagaagcctgttacttgctttagctgctggccttgcagtggcaaggccccctaacatcgtgctgatctttgcagatgacttgggatatggggatcttggttgttatggccacccatcaagcacaactcccaatctggatcagttggctgcaggaggtctgaggtttacagacttttatgttccagtctccctgtgcactccttctcgggctgccctgcttactgggaggctccctgtgagaatgggtatgtaccctggagtgttggtcccatccagcaggggagggctgcccctggaagaggtgacagtggcagaggtgctggcagcacgaggctatctgactggcatggcaggcaagtggcacctgggtgtagggccagagggtgctttcctgcctccccatcagggctttcataggtttctgggaatcccatactctcatgaccaaggaccctgccagaacctcacctgtttcccccctgcaacaccatgtgatgggggctgtgatcaaggtctggttcctataccactgcttgctaatctttcagtggaagctcaaccaccctggctgcctggcttggaggctagatacatggccttcgcacatgatctgatggcagatgcccagagacaagataggcctttcttcctctactatgcatctcaccacacccactatcctcagttctcaggccaatcatttgctgagcgtagtggcaggggcccatttggggacagtttgatggaactggatgccgcagttggtaccctcatgacagcaataggggacttaggtttgctggaggaaacattggtaattttcacagctgataatggccctgagacaatgagaatgtctaggggaggctgctctggtcttctgaggtgtggtaaagggactacatatgagggaggagtgagggaaccagctcttgccttttggccaggtcacatagcccctggagttacacatgaactagcttcttccctggacttgcttcctacactggcagccctggcaggtgcccctctccctaatgtaactttagatggatttgacctctctccactacttttagggacagggaaaagtccaaggcagtccttattcttctatccttcctacccagatgaggtgaggggtgtttttgccgtgaggactgggaaatacaaagctcatttttttacccagggatcagctcattcagacaccacagctgatcctgcctgtcatgccagcagtagcttgacagcacatgagcctcccttactgtatgacctgagcaaggacccaggggagaactataacctgcttgggggggttgctggggccaccccagaagtgcttcaggcactaaagcagctgcaactgcttaaagcacagttggatgctgcagtgacctttggcccttcccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctatggctgccccagccctggggctggtgtgtggcagatgccctgagctgggcctggtgctgcttctcctgctgctgagcctcctgtgtggtgctgctggctctcaggaagcagggacaggagcaggagcaggttctctggctggctcatgcggttgtgggaccccccagaggccaggggctcatgggtcctctgcagctgcccacaggtactcaagggaagcaaatgcccctggccccgtacctggggaaaggcaacttgctcactccaagatggttcctatccctgcaggagtttttactatgggaactgatgaccctcagatcaagcaggatggtgaagcaccagctaggagagtcacaattgatgccttctatatggatgcctatgaagtgtcaaacacagaatttgagaaatttgtaaacagcactggataccttacagaggctgagaaatttggtgacagttttgtttttgaaggcatgctaagtgagcaggtgaagaccaatatccaacaggcagtggctgcagccccctggtggctgcctgttaaaggagccaattggagacacccagagggaccagactcaactatcctccacaggcctgaccaccctgtgctgcatgtgtcctggaatgatgcagtggcatactgcacctgggctgggaaaaggttaccaacagaggcagaatgggagtattcctgccggggtggactgcacaacagactgttcccctggggcaataagctgcaacctaaaggacagcattatgccaatatttggcagggagagttcccagtcacaaacactggtgaggatggcttccagggaactgcccctgtggatgctttcccacccaatggctatgggttgtacaatatagttgggaatgcctgggagtggacttctgactggtggacggtccatcacagtgtggaagagacactgaacccaaaggggcccccctcaggcaaggacagagtcaagaaaggtggctcttatatgtgtcacagaagctattgctacagatataggtgtgctgcaagaagtcagaacacccctgacagctcagctagcaatctgggatttagatgtgcagcagatagactccccaccatggactgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactgg

SEQ ID NO: 71 - DNA, 6335 bp, Artificial Sequence
  Synthetic polynucleotide
gtcaggtggcacttttcggggaaatgtggcatgcctgcatttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcacctaggtcattctggcctccccctccctcaaggccagtcattctggcctgtccttccccgaaggccagtcattctggcctccccctcccccaaggccagtcattctggccttcccctcccttaaggccagagtactatcgattcacacaaaaaaccaacacactattgcaatgaaaataaatttcctttattaagcttaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaaggctagcgccgccaccatgagcatgggcgcccccagaagcctgttacttgctttagctgctggccttgcagtggcaaggccccctaacatcgtgctgatctttgcagatgacttgggatatggggatcttggttgttatggccacccatcaagcacaactcccaatctggatcagttggctgcaggaggtctgaggtttacagacttttatgttccagtctccctgtgcactccttctcgggctgccctgcttactgggaggctccctgtgagaatgggtatgtaccctggagtgttggtcccatccagcaggggagggctgcccctggaagaggtgacagtggcagaggtgctggcagcacgaggctatctgactggcatggcaggcaagtggcacctgggtgtagggccagagggtgctttcctgcctccccatcagggctttcataggtttctgggaatcccatactctcatgaccaaggaccctgccagaacctcacctgtttcccccctgcaacaccatgtgatgggggctgtgatcaaggtctggttcctataccactgcttgctaatctttcagtggaagctcaaccaccctggctgcctggcttggaggctagatacatggccttcgcacatgatctgatggcagatgcccagagacaagataggcctttcttcctctactatgcatctcaccacacccactatcctcagttctcaggccaatcatttgctgagcgtagtggcaggggcccatttggggacagtttgatggaactggatgccgcagttggtaccctcatgacagcaataggggacttaggtttgctggaggaaacattggtaattttcacagctgataatggccctgagacaatgagaatgtctaggggaggctgctctggtcttctgaggtgtggtaaagggactacatatgagggaggagtgagggaaccagctcttgccttttggccaggtcacatagcccctggagttacacatgaactagcttcttccctggacttgcttcctacactggcagccctggcaggtgcccctctccctaatgtaactttagatggatttgacctctctccactacttttagggacagggaaaagtccaaggcagtccttattcttctatccttcctacccagatgaggtgaggggtgtttttgccgtgaggactgggaaatacaaagctcatttttttacccagggatcagctcattcagacaccacagctgatcctgcctgtcatgccagcagtagcttgacagcacatgagcctcccttactgtatgacctgagcaaggacccaggggagaactataacctgcttgggggggttgctggggccaccccagaagtgcttcaggcactaaagcagctgcaactgcttaaagcacagttggatgctgcagtgacctttggcccttcccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctatggctgccccagccctggggctggtgtgtggcagatgccctgagctgggcctggtgctgcttctcctgctgctgagcctcctgtgtggtgctgctggctctcaggaagcagggacaggagcaggagcaggttctctggctggctcatgcggttgtgggaccccccagaggccaggggctcatgggtcctctgcagctgcccacaggtactcaagggaagcaaatgcccctggccccgtacctggggaaaggcaacttgctcactccaagatggttcctatccctgcaggagtttttactatgggaactgatgaccctcagatcaagcaggatggtgaagcaccagctaggagagtcacaattgatgccttctatatggatgcctatgaagtgtcaaacacagaatttgagaaatttgtaaacagcactggataccttacagaggctgagaaatttggtgacagttttgtttttgaaggcatgctaagtgagcaggtgaagaccaatatccaacaggcagtggctgcagccccctggtggctgcctgttaaaggagccaattggagacacccagagggaccagactcaactatcctccacaggcctgaccaccctgtgctgcatgtgtcctggaatgatgcagtggcatactgcacctgggctgggaaaaggttaccaacagaggcagaatgggagtattcctgccggggtggactgcacaacagactgttcccctggggcaataagctgcaacctaaaggacagcattatgccaatatttggcagggagagttcccagtcacaaacactggtgaggatggcttccagggaactgcccctgtggatgctttcccacccaatggctatgggttgtacaatatagttgggaatgcctgggagtggacttctgactggtggacggtccatcacagtgtggaagagacactgaacccaaaggggcccccctcaggcaaggacagagtcaagaaaggtggctcttatatgtgtcacagaagctattgctacagatataggtgtgctgcaagaagtcagaacacccctgacagctcagctagcaatctgggatttagatgtgcagcagatagactccccaccatggactgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatccccgggtaccgaggacgaattctctagatatcgctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctgagggcccaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgatatcaagcttgaattcgtt

SEQ ID NO: 72 - DNA, 1527 bp, Artificial Sequence
  Synthetic polynucleotide
atgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgctgccacccaggctgcacacccagacctgcctgctgtcactgccccgacccacacgcc

SEQ ID NO: 73 - DNA, 237 bp, Homo sapiens
ggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgag

SEQ ID NO: 74 - DNA, 1833 bp, Artificial Sequence
  Synthetic polynucleotide
atgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgagtga

SEQ ID NO: 75 - DNA, 3698 bp, Artificial Sequence
  Synthetic polynucleotide
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgagtgaactagtaacttgtttattgcagcttataatggttacaaataaagcaatagcatcacaaatttcacaaataaagcatttttttcactgcattctagttgtggtttgtccaaactcatcaatgtatctta

SEQ ID NO: 76 - DNA, 4231 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgagtgaactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 77 - DNA, 6073 bp, Artificial Sequence
  Synthetic polynucleotide
tttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaatgatatcaagcttgaattcgtgtcaggtggcacttttcggggaaatgtggcatgcctgcatttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgctgccacccaggctgcacacccagacctgcctgctgtcactgccccgacccacacgccggcagcggagctactaacttcagcctgctgaagcaggctggagacgtggaggagaaccctggacctggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgagtgaactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatccccgggtaccgaggacgaattctctagatatcgctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctgagggcccaaatgtaatcacctggctcaccttcgggtgggcctttctgcgttgctggcgtt

SEQ ID NO: 78 - DNA, 42 bp, Artificial Sequence
  Synthetic nucleic acid sequence
ggaaaaccaataccaaaccctctattaggattggactcaaca

SEQ ID NO: 79 - DNA, 3458 bp, Artificial Sequence
  Synthetic nucleic acid sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgccggaaaaccaataccaaaccctctattaggattggactcaacataagattctagagtcgagccgcggactagtaacttgtttattgcagcttataatggttacaaataaagcaatagcatcacaaatttcacaaataaagcatttttttcactgcattctagttgtggtttgtccaaactcatcaatgtatctta

SEQ ID NO: 80 - DNA, 3991 bp, Artificial Sequence
  Synthetic nucleic acid sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgccggaaaaccaataccaaaccctctattaggattggactcaacataagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 81 - DNA, 6654 bp, Artificial Sequence
  Synthetic nucleic acid sequence
cgccagggttttcccagtcacgacgttgtaaaacgacggccagtgccaagcttgcatgcctgcatttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatgtctatgggggctcctcgctccctgctgctggcactggccgccgggctggctgtcgcaagaccacctaatatcgtcctgatttttgcagacgatctgggatacggcgacctgggatgctatggccacccaagctccaccacacccaacctggaccagctggcagcaggaggcctgcggttcaccgacttctacgtgccagtgagcctgtgcaccccctccagagccgccctgctgacaggcaggctgccagtgcgcatgggcatgtatcctggcgtgctggtgccatctagcaggggcggcctgccactggaggaggtgaccgtggcagaggtgctggcagccagaggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggcggatgcagcggcctgctgagatgtggcaagggaaccacatacgagggaggcgtgcgcgagcctgccctggcattttggccaggacacatcgcacctggagtgacccacgagctggcctcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagcagatccagcatgccacgccagctcctctctgaccgcacacgagccacctctgctgtacgacctgtccaaggatcccggcgagaactataatctgctgggaggagtggcaggagcaacccctgaggtgctgcaggccctgaagcagctgcagctgctgaaggcacagctggacgcagcagtgacattcggcccaagccaggtggccagaggcgaggatcccgccctgcagatctgttgccaccccggctgcaccccaagacctgcctgttgccattgccccgacccacacgccggaaaaccaataccaaaccctctattaggattggactcaacataagattctagagtcgagccgcggactagtaacttgtttattgcagcttataatggttacaaataaagcaatagcatcacaaatttcacaaataaagcatttttttcactgcattctagttgtggtttgtccaaactcatcaatgtatcttaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatccccgggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggcgaacttttgctgagttgaaggatcagatcacgcatcttcccgacaacgcagaccgttccgtggcaaagcaaaagttcaaaatcagtaaccgtcagtgccgataagttcaaagttaaacctggtgttgataccaacattgaaacgctgatcgaaaacgcgctgaaaaacgctgctgaatgtgcgagcttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgatccgtcgagaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgcattcgccattcaggctgcgcaactgttgggaagggcgatcggtgcgggcctcttcgctattacgccagctggcgaaagggggatgtgctgcaaggcgattaagttgggtaa

(19) United States
(12) Patent Application Publication
(10) Pub. No.: US 2022/0204991 A1
(43) Pub. Date: Jun. 30, 2022

(54) Adeno-Associated Virus Compositions for ARSA Gene Transfer and Methods of Use Thereof

(71) Applicant: Homology Medicines, Inc., Bedford, MA (US)

(72) Inventors: Thia Baboval ST. MARTIN, Bedford, MA (US); Albert Barnes SEYMOUR, Bedford, MA (US); Hillard RUBIN, Bedford, MA (US)

(21) Appl. No.: 17/643,631

(22) Filed: Dec. 10, 2021

Related U.S. Application Data
(63) Continuation of application No. PCT/US2020/036846, filed on Jun. 9, 2020.
(60) Provisional application No. 63/010,970, filed on Apr. 16, 2020.
(60) Provisional application No. 62/987,858, filed on Mar. 10, 2020.
(60) Provisional application No. 62/960,487, filed on Jan. 13, 2020.
(60) Provisional application No. 62/915,523, filed on Oct. 15, 2019.
(60) Provisional application No. 62/866,374, filed on Jun. 25, 2019.
(60) Provisional application No. 62/859,539, filed on Jun. 10, 2019.

(51) Int. Cl. C12N 15/86; C12N 9/16; A61P 25/28; A61K 48/00

(52) U.S. Cl. CPC C12N 15/86; C12N 9/16; C12Y 301/06001; A61K 48/005; A61P 25/28

(57) ABSTRACT

Provided herein are adeno-associated virus (AAV) compositions that can express an arylsulfatase A (ARSA) polypeptide in a cell, thereby restoring the ARSA gene function. Also provided are methods of using the AAV compositions, and packaging systems for making the AAV compositions.

### CROSS-REFERENCE TO RELATED APPLICATIONS

This application is a continuation of International Application No. PCT/US2020/036846, filed Jun. 9, 2020, which claims the benefit of U.S. Provisional Application Nos.: 62/859,539, filed Jun. 10, 2019, 62/866,374, filed Jun. 25, 2019, 62/915,523, filed Oct. 15, 2019, 62/960,487, filed Jan. 13, 2020, 62/987,858, filed Mar. 10, 2020, and 63/010,970, filed Apr. 16, 2020, each of which is hereby incorporated by reference in its entirety.

### SEQUENCE LISTING

This application contains a sequence listing which has been submitted electronically in ASCII format and is hereby incorporated by reference in its entirety (said ASCII copy, created on Dec. 10, 2021, is named “HMW-030US_ST25.txt” and is 295,995 bytes in size).

### BACKGROUND

Metachromatic leukodystrophy (MLD) is a fatal lysosomal storage disorder with a high unmet medical need. This neurodegenerative disease occurs in three forms (late infantile, juvenile and adult) and is due to a deficiency in the lysosomal enzyme arylsulfatase-A (ARSA). ARSA is located in cellular structures called lysosomes, where it helps to break down sulfatides. The lack of this enzyme leads to a large accumulation of sulfatides in the brain, spinal cord and peripheral organs, which results in severe damage of myelin, the main protective layer of the nerve fibers. Sulfatide accumulation in myelin-producing cells causes progressive destruction of white matter throughout the nervous system, including in the brain, spinal cord, and the nerves connecting the brain and spinal cord to muscles and sensory cells that detect sensations such as touch, pain, heat, and sound. Accordingly, MLD is characterized by progressive axonal demyelination of the central nervous system, and then the peripheral nervous system. This results in loss of acquired functions and/or skills, hypotonia, ataxia, seizures, blindness, hearing loss, and in untimely death.

In people with metachromatic leukodystrophy, white matter damage causes progressive deterioration of intellectual functions and motor skills, such as the ability to walk. Affected individuals also develop loss of sensation in the extremities, incontinence, seizures, paralysis, an inability to speak, blindness, and hearing loss. Eventually, such individuals lose awareness of their surroundings and become unresponsive. While neurological problems are the primary feature of metachromatic leukodystrophy, effects of sulfatide accumulation on other organs and tissues have been reported, most often involving the gallbladder.

MLD can be managed with several treatments. For example, medications to reduce signs and symptoms of MLD and to relieve associated pain. Hematopoietic stem cell transplants have been shown to delay the progression of MLD by introducing healthy cells to help replace diseased ones. Other treatments include physical, occupational, and speech therapy to promote muscle and joint flexibility and maintain range of motion. However, there is no cure for MLD.

Most individuals with MLD have mutations in the arylsulfatase A (ARSA) gene, and over 110 distinct ARSA mutations have been identified that cause MLD. Carrier mutations have been found in 1 in 100 people, and affect 1 in 40,000 live births in U.S., or 1 in 160,000 worldwide.

Gene therapy provides a unique opportunity to cure MLD. Retroviral vectors, including lentiviral vectors, are capable of integrating nucleic acids into host cell genomes, raising safety concerns due to their non-targeted insertion into the genome. For example, there is a risk of the vector disrupting a tumor suppressor gene or activating an oncogene, thereby causing a malignancy. Indeed, in a clinical trial for treating X-linked severe combined immunodeficiency (SCID) by transducing CD34⁺ bone marrow precursors with a gammaretroviral vector, four out of ten patients developed leukemia (Hacein-Bey-Abina et al; J Clin Invest. (2008) 118(9):3132-42). Non-integrating vectors, on the other hand, often suffer insufficient expression level or inadequate duration of expression in vivo.

Accordingly, there is a need in the art for improved gene therapy compositions and methods that can efficiently and safely restore ARSA gene function in MLD patients.

### SUMMARY

Provided herein are adeno-associated virus (AAV) compositions that can restore ARSA gene function in cells, and methods for using the same to treat diseases associated with reduction of ARSA gene function (e.g., MLD). Also provided are packaging systems for making the adeno-associated virus compositions.

Accordingly, in one aspect, the instant disclosure provides a method for expressing an arylsulfatase A (ARSA) polypeptide in a cell, the method comprising transducing the cell with a recombinant adeno-associated virus (rAAV) comprising: (a) an AAV capsid comprising an AAV capsid protein (e.g., a Clade F capsid protein); and (b) a transfer genome comprising a transcriptional regulatory element operably linked to a silently altered ARSA coding sequence.

In certain embodiments, the cell is a neuron and/or a glial cell. In certain embodiments, the cell is a neuron and/or a glial cell of the central nervous system and/or the peripheral nervous system. In certain embodiments, the cell is a cell of a central nervous system region selected from the group consisting of the spinal cord, the motor cortex, the sensory cortex, the hippocampus, the putamen, the cerebellum optionally the cerebellar nuclei, and any combination thereof. In certain embodiments, the cell is a cell selected from the group consisting of a motor neuron, an astrocyte, an oligodendrocyte, a cell of the cerebral cortex in the central nervous system, a sensory neuron of the peripheral nervous system, a Schwann cell, and any combination thereof. In certain embodiments, the cell is in a mammalian subject and the AAV is administered to the subject in an amount effective to transduce the cell in the subject.

In another aspect, the instant disclosure provides a method for treating a subject having metachromatic leukodystrophy (MLD), the method comprising administering to the subject an effective amount of an rAAV comprising: (a) an AAV capsid comprising an AAV capsid protein (e.g., a Clade F capsid protein); and (b) a transfer genome comprising a transcriptional regulatory element operably linked to a silently altered ARSA coding sequence.

In certain embodiments, the silently altered ARSA coding sequence encodes an amino acid sequence set forth in SEQ ID NO: 23. In certain embodiments, the silently altered ARSA coding sequence comprises the nucleotide sequence set forth in SEQ ID NO: 14, 62, or 72.

In certain embodiments, the transcriptional regulatory element comprises one or more of the elements selected from the group consisting of a cytomegalovirus (CMV) enhancer element, a chicken-β-actin (CBA) promoter, a small chicken-β-actin (SmCBA) promoter, a calmodulin 1 (CALM1) promoter, a proteolipid protein 1 (PLP1) promoter, a glial fibrillary acidic protein (GFAP) promoter, a synapsin 2 (SYN2) promoter, a metallothionein 3 (MT3) promoter, and any combination thereof. In certain embodiments, the transcriptional regulatory element comprises a nucleotide sequence at least 90% identical to a sequence selected from the group consisting of SEQ ID NO: 25, 32, 36, 54, 55, and 58. In certain embodiments, the transcriptional regulatory element comprises a nucleotide sequence selected from the group consisting of SEQ ID NO: 25, 32, 36, 54, 55, and 58. In certain embodiments, the transcriptional regulatory element comprises from 5′ to 3′ the nucleotide sequences set forth in SEQ ID NO: 58, 25, and 32. In certain embodiments, the transcriptional regulatory element comprises the nucleotide sequence set forth in SEQ ID NO: 36.

In certain embodiments, the transfer genome further comprises a polyadenylation sequence 3′ to the silently altered ARSA coding sequence. In certain embodiments, the polyadenylation sequence is an exogenous polyadenylation sequence. In certain embodiments, the exogenous polyadenylation sequence is an SV40 polyadenylation sequence. In certain embodiments, the SV40 polyadenylation sequence comprises the nucleotide sequence set forth in SEQ ID NO: 42.

In certain embodiments, the transfer genome further comprises a stuffer sequence. In certain embodiments, the transfer genome further comprises a stuffer sequence 3′ to the silently altered ARSA coding sequence. In certain embodiments, the stuffer sequence is 3′ to the polyadenylation sequence.

In certain embodiments, the transfer genome comprises a sequence selected from the group consisting of SEQ ID NO: 41, 44, 46, 65, 67, and 75.

In certain embodiments, the transfer genome further comprises a 5′ inverted terminal repeat (5′ ITR) nucleotide sequence 5′ of the genome, and a 3′ inverted terminal repeat (3′ ITR) nucleotide sequence 3′ of the genome. In certain embodiments, the 5′ ITR nucleotide sequence has at least 95% sequence identity to SEQ ID NO: 18, and the 3′ ITR nucleotide sequence has at least 95% sequence identity to SEQ ID NO: 19. In certain embodiments, the 5′ ITR nucleotide sequence has at least 95% sequence identity to SEQ ID NO: 26, and the 3′ ITR nucleotide sequence has at least 95% sequence identity to SEQ ID NO: 27. In certain embodiments, the 5′ ITR nucleotide sequence has at least 95% sequence identity to SEQ ID NO: 18, and the 3′ ITR nucleotide sequence has at least 95% sequence identity to SEQ ID NO: 57.

In certain embodiments, the transfer genome comprises a nucleotide sequence selected from the group consisting of SEQ ID NO: 47, 48, 49, 68, 69, and 76.

In certain embodiments, metachromatic leukodystrophy is associated with an arylsulfatase A (ARSA) gene mutation. In certain embodiments, the subject is a human subject.

In certain embodiments, the capsid protein comprises an amino acid sequence having at least 95% sequence identity with the amino acid sequence of amino acids 203-736 of SEQ ID NO: 2, 3, 4, 6, 7, 10, 11, 12, 13, 15, 16, or 17. In certain embodiments, the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 203-736 of SEQ ID NO: 2, 3, 4, 6, 7, 10, 11, 12, 13, 15, 16, or 17.

In certain embodiments, the capsid protein comprises an amino acid sequence having at least 95% sequence identity with the amino acid sequence of amino acids 138-736 of SEQ ID NO: 2, 3, 4, 5, 6, 7, 9, 10, 11, 12, 13, 15, 16, or 17. In certain embodiments, the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 138-736 of SEQ ID NO: 2, 3, 4, 5, 6, 7, 9, 10, 11, 12, 13, 15, 16, or 17.

In certain embodiments, the capsid protein comprises an amino acid sequence having at least 95% sequence identity with the amino acid sequence of amino acids 1-736 of SEQ ID NO: 2, 3, 4, 5, 6, 7, 8, 9, 10, 11, 12, 13, 15, 16, or 17. In certain embodiments, the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (0 the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 1-736 of SEQ ID NO: 2, 3, 4, 5, 6, 7, 8, 9, 10, 11, 12, 13, 15, 16, or 17.

In another aspect, the instant disclosure provides an rAAV comprising: (a) an AAV capsid comprising an AAV capsid protein (e.g., a Clade F capsid protein); and (b) a transfer genome comprising a transcriptional regulatory element operably linked to a silently altered ARSA coding sequence.

In certain embodiments, the silently altered ARSA coding sequence encodes an amino acid sequence set forth in SEQ ID NO: 23. In certain embodiments, the silently altered ARSA coding sequence comprises the nucleotide sequence set forth in SEQ ID NO: 14. In certain embodiments, the silently altered ARSA coding sequence comprises the nucleotide sequence set forth in SEQ ID NO: 62 or 72.

In certain embodiments, the transcriptional regulatory element comprises one or more of the elements selected from the group consisting of a cytomegalovirus (CMV) enhancer element, a chicken-β-actin (CBA) promoter, a small chicken-β-actin (SmCBA) promoter, a calmodulin 1 (CALM1) promoter, a proteolipid protein 1 (PLP1) promoter, a glial fibrillary acidic protein (GFAP) promoter, a synapsin 2 (SYN2) promoter, a metallothionein 3 (MT3) promoter, and any combination thereof. In certain embodiments, the transcriptional regulatory element comprises a nucleotide sequence at least 90% identical to a sequence selected from the group consisting of SEQ ID NO: 25, 32, 36, 54, 55, and 58. In certain embodiments, the transcriptional regulatory element comprises a nucleotide sequence selected from the group consisting of SEQ ID NO: 25, 32, 36, 54, 55, and 58. In certain embodiments, the transcriptional regulatory element comprises from 5′ to 3′ the nucleotide sequences set forth in SEQ ID NO: 58, 25, and 32. In certain embodiments, the transcriptional regulatory element comprises the nucleotide sequence set forth in SEQ ID NO: 36

In certain embodiments, the transfer genome further comprises a polyadenylation sequence 3′ to the silently altered ARSA coding sequence. In certain embodiments, the polyadenylation sequence is an exogenous polyadenylation sequence. In certain embodiments, the exogenous polyadenylation sequence is an SV40 polyadenylation sequence. In certain embodiments, the SV40 polyadenylation sequence comprises the nucleotide sequence set forth in SEQ ID NO: 42.

In certain embodiments, the transfer genome comprises a sequence selected from the group consisting of SEQ ID NO: 41, 44, 46, 65, 67, and 75.

In certain embodiments, the transfer genome further comprises a 5′ inverted terminal repeat (5′ ITR) nucleotide sequence 5′ of the genome, and a 3′ inverted terminal repeat (3′ ITR) nucleotide sequence 3′ of the genome. In certain embodiments, the 5′ ITR nucleotide sequence has at least 95% sequence identity to SEQ ID NO: 18, and the 3′ ITR nucleotide sequence has at least 95% sequence identity to SEQ ID NO: 19.

In certain embodiments, the transfer genome comprises a nucleotide sequence selected from the group consisting of SEQ ID NO: 47, 48, 49, 68, 69, and 76. In certain embodiments, the nucleotide sequence of the transfer genome consists of a nucleotide sequence selected from the group consisting of SEQ ID NO: 47, 48, 49, 68, 69, and 76. In certain embodiments, the nucleotide sequence of the transfer genome consists of the nucleotide sequence set forth in SEQ ID NO: 48.

In certain embodiments, the capsid protein comprises an amino acid sequence having at least 95% sequence identity with the amino acid sequence of amino acids 203-736 of SEQ ID NO: 2, 3, 4, 6, 7, 10, 11, 12, 13, 15, 16, or 17. In certain embodiments, the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 203-736 of SEQ ID NO: 2, 3, 4, 6, 7, 10, 11, 12, 13, 15, 16, or 17.

In certain embodiments, the capsid protein comprises an amino acid sequence having at least 95% sequence identity with the amino acid sequence of amino acids 138-736 of SEQ ID NO: 2, 3, 4, 5, 6, 7, 9, 10, 11, 12, 13, 15, 16, or 17. In certain embodiments, the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 138-736 of SEQ ID NO: 2, 3, 4, 5, 6, 7, 9, 10, 11, 12, 13, 15, 16, or 17.

In certain embodiments, the capsid protein comprises an amino acid sequence having at least 95% sequence identity with the amino acid sequence of amino acids 1-736 of SEQ ID NO: 2, 3, 4, 5, 6, 7, 8, 9, 10, 11, 12, 13, 15, 16, or 17. In certain embodiments, the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (0 the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 1-736 of SEQ ID NO: 2, 3, 4, 5, 6, 7, 8, 9, 10, 11, 12, 13, 15, 16, or 17.

In another aspect, the instant disclosure provides a pharmaceutical composition comprising an rAAV described herein.

In another aspect, the instant disclosure provides a polynucleotide comprising the nucleic acid sequence set forth in SEQ ID NO: 14, 62, and 72.

In another aspect, the instant disclosure provides a packaging system for preparation of an rAAV, wherein the packaging system comprises (a) a first nucleotide sequence encoding one or more AAV Rep proteins; (b) a second nucleotide sequence encoding a capsid protein of the AAV of any one of claims 41 to 71; and (c) a third nucleotide sequence comprising an rAAV genome sequence of the AAV of any one of claims 41 to 71.

In certain embodiments, the packaging system comprises a first vector comprising the first nucleotide sequence and the second nucleotide sequence, and a second vector comprising the third nucleotide sequence.

In certain embodiments, the packaging system further comprises a forth nucleotide sequence comprising one or more helper virus genes. In certain embodiments, the forth nucleotide sequence is comprised within a third vector. In certain embodiments, the forth nucleotide sequence comprises one or more genes from a virus selected from the group consisting of adenovirus, herpes virus, vaccinia virus, and cytomegalovirus (CMV).

In certain embodiments, the first vector, second vector, and/or the third vector is a plasmid.

In another aspect, the instant disclosure provides a method for recombinant preparation of an rAAV, the method comprising introducing a packaging system described herein into a cell under conditions whereby the rAAV is produced.

In another aspect, the instant disclosure provides an rAAV described herein, for use in a method for expressing an arylsulfatase A (ARSA) polypeptide in a cell as described herein.

In another aspect, the instant disclosure provides an rAAV described herein, for use in a method for treating a subject having metachromatic leukodystrophy (MLD) as described herein.

### BRIEF DESCRIPTION OF THE DRAWINGS

FIGS. 1A, 1B, 1C, and 1D are vector maps of the T-001, pHMI-5000, pHMI-5003, and pHMI-hARSA1-TC-002 vectors, respectively.

FIGS. 2A, 2B, and 2C. FIG. 2A is a graph showing the quantification of total pixel intensity derived from LAMP-1 immunoreactivity investigated by immunohistochemistry using an anti-LAMP-1 antibody in ARSA(−/−) mice treated with vehicle control or pHMI-5000 packaged in AAVHSC15 capsid (dWM: dorsal white matter; vWM: ventral white matter; and vGM: ventral gray matter). FIG. 2B is a graph showing the level of C18:0 sulfatides measured in the brains of control group mice (WT/Het) and ARSA(−/−) mice over time. FIG. 2C is a graph showing the change in the level of sulfatides (as fold over age-matched wild type controls) in ARSA(−/−) mice that were treated with pHMI-hARSA1-TC-002 packaged in AAVHSC15 capsid at a dose of 4e13 vg/kg (Dose-4), or vehicle control. FIG. 2D is a set of graphs showing the change in the levels of C18:0 and C18:1 sulfatide isoforms (as fold over age-matched wild type controls) in the forebrain, midbrain, and hindbrain of ARSA(−/−) mice that were treated with pHMI-5000 packaged in AAVHSC15 capsid at a dose of 4e13 vg/kg or 6e13 vg/kg, or vehicle control. FIG. 2E is a set of graphs showing the change in the levels of C18:0 and C18:1 sulfatide isoforms (as fold over age-matched wild type controls) in the forebrain, midbrain, and hindbrain of ARSA(−/−) mice that were treated with pHMI-5000 packaged in AAVHSC15 capsid at a dose of 4e13 vg/kg, or vehicle control. FIG. 2F is a set of graphs showing the change in the levels of C24:0 and C24:1 sulfatide isoforms (as fold over age-matched wild type controls) in the forebrain, midbrain, and hindbrain of ARSA(−/−) mice that were treated with pHMI-5000 packaged in AAVHSC15 capsid at a dose of 4e13 vg/kg, or vehicle control. FIG. 2G is a set of graphs showing the change in the level of total sulfatide isoforms (as fold over age-matched wild type controls) in the forebrain, midbrain, and hindbrain of ARSA(−/−) mice that were treated with pHMI-5000 packaged in AAVHSC15 capsid at a dose of 4e13 vg/kg, or vehicle control.

FIGS. 3A and 3B. FIG. 3A is a graph showing the level of myelin and lymphocyte protein (MAL) mRNA transcript measured at four weeks in control group mice (WT/Het) and ARSA(−/−) mice. FIG. 3B is a graph showing the level of MAL transcript detected in ARSA(−/−) mice treated with pHMI-5000 packaged in AAVHSC15 capsid at a dose of 4e13 vg/kg (Dose-4) compared to age-matched wild type mice and vehicle treated ARSA(−/−) mice. FIG. 3C is a graph showing the MAL transcript copy number detected in wild type mice or ARSA(−/−) mice, 12 or 52 weeks after administration of 4e13 vg/kg of pHMI-5000 packaged in AAVHSC15 capsid or vehicle control.

FIG. 4 is a plot showing the correlation between the number of vector genomes per transduced cell in the brains of ARSA(−/−) mice, and the number of copies of hARSA per ng of cDNA.

FIG. 5 is a graph showing the number of vector genomes per transduced cell in the brains of ARSA(−/−) mice after intravenous administration of transfer vector pHMI-5000 packaged in either AAV9 or AAVHSC15 capsid, in each case administered at a dose of 2e13 vg/kg.

FIG. 6 is a graph showing the percent of normal human ARSA enzyme activity levels measured in the brain of ARSA(−/−) mice after intravenous administration of transfer vector pHMI-5000 packaged in either AAV9 or AAVHSC15 capsid and administered at the indicated doses.

FIG. 7 is a graph showing the number of vector genomes per cell in the brain in ARSA(−/−) mice intravenously administered transfer vector pHMI-5000 packaged in either AAV9 or AAVHSC15, in each case at a dose of 4e13 vg/kg.

FIG. 8 is a graph showing the percent of normal human ARSA enzyme activity in hindbrain and midbrain following intravenous (IV) or intrathecal (IT) administration of transfer vector pHMI-5000 packaged in AAVHSC15.

FIGS. 9A,9B, 9C, and 9D. FIG. 9A is a graph showing the percentage of normal hARSA activity achieved in the brain after intravenous administration of transfer vector pHMI-5000 packaged in AAVHSC15 capsid to ARSA(−/−) mice at the indicated doses.

FIG. 9B is a graph showing the number of vector genomes per cell in brains of ARSA(−/−) mice after intravenous administration of transfer vector pHMI-5000 packaged in AAVHSC15 capsid at the indicated doses. FIG. 9C is a graph showing the level of hARSA enzyme activity in neonate ARSA(−/−) mice dosed with 4e13 vg/kg of pHMI-5000 packaged in AAVHSC15 capsid over the course of 12 weeks post-dosing. FIG. 9D is a graph showing the level of ARSA enzyme activity (via hARSA transcript analysis) in the brains of adult ARSA(−/−) mice dosed with 4e13 vg/kg of pHMI-5000 packaged in AAVHSC15 capsid.

FIG. 9E is a graph showing the number of vector genomes per ug of genomic DNA in brains of ARSA(−/−) mice administered a single intravenous 4e13 vg/kg dose of pHMI-5000 packaged in AAVHSC15 capsid. FIG. 9F is a graph showing the number of copies of ARSA transcript per ng of RNA in brains of ARSA(−/−) mice administered a single intravenous 4e13 vg/kg dose of pHMI-5000 packaged in AAVHSC15 capsid.

FIGS. 10A and 10B are vector maps of the TC-013.pHMIA2 and TC-015.pKITR vectors, respectively.

FIG. 11 is a graph showing the number of viral genomes transduced per cell in the brains of mice ARSA(−/−) mice administered transfer vectors pHMI-5000 (CBA promoter), TC-013.pHMIA2 (CALM1 promoter), and TC-015.pKITR (smCBA promoter), in each case packaged in AAVHSC15 capsid and administered intravenously at a dose of 4e13 vg/kg.

FIG. 12 is a graph showing the percent of normal human ARSA enzyme activity detected in the brains of mice ARSA(−/−) mice administered transfer vectors pHMI-5000 (CBA promoter) and TC-015.pKITR (smCBA promoter), in each case packaged in AAVHSC15 capsid and administered intravenously at a dose of 4e13 vg/kg.

FIG. 13 are photographs of immunoblots showing the expression of hARSA in brains of mice using an anti-hARSA antibody. ARSA(−/−) mice were administered transfer vectors pHMI-5000 (CBA promoter) and TC-015.pKITR (smCBA promoter), in each case packaged in AAVHSC15 capsid, and administered intravenously at a dose of 4e13 vg/kg and 8e13 vg/kg, respectively (n=5 mice for each vector).

FIG. 14 is a vector map of the transfer vector pHMI-5004.

FIG. 15 is a vector map of the transfer vector pHMI-5005.

FIG. 16 is a graph showing alanine transaminase (ALT) levels in non-human primates treated with pHMI-5005 packaged in AAVHSC15 capsid at the dose indicated doses, or treated with vehicle control.

FIG. 17 is a graph showing ARSA activity in the central nervous system (CNS) and cerebrospinal fluid (CSF) of non-human primates dosed with pHMI-5005 packaged in AAVHSC15 capsid.

### DETAILED DESCRIPTION

Provided herein are adeno-associated virus (AAV) compositions that can restore ARSA gene function in cells, and methods for using the same to treat diseases associated with reduction of ARSA gene function (e.g., MLD). Also provided are packaging systems for making the adeno-associated virus compositions.

### I. DEFINITIONS

As used herein, the term “replication-defective adeno-associated virus” refers to an AAV comprising a genome lacking Rep and Cap genes.

As used herein, the term “ARSA gene” refers to the arylsulfatase A gene. The human ARSA gene is identified by National Center for Biotechnology Information (NCBI) Gene ID 410. An exemplary nucleotide sequence of a ARSA mRNA is provided as SEQ ID NO:14. An exemplary amino acid sequence of a ARSA polypeptide is provided as SEQ ID NO:23.

As used herein, the term “transfer genome” refers to a recombinant AAV genome comprising a coding sequence operably linked to an exogenous transcriptional regulatory element that mediates expression of the coding sequence when the transfer genome is introduced into a cell. In certain embodiments, the transfer genome does not integrate in the chromosomal DNA of the cell. The skilled artisan will appreciate that the portion of a transfer genome comprising the transcriptional regulatory element operably linked to an ARSA coding sequence can be in the sense or antisense orientation relative to direction of transcription of the ARSA coding sequence.

As used herein, the term “Clade F capsid protein” refers to an AAV VP1, VP2, or VP3 capsid protein that has at least 90% identity with the VP1, VP2, or VP3 amino acid sequences set forth, respectively, in amino acids 1-736, 138-736, and 203-736 of SEQ ID NO: 1 herein.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Only internal gaps are included in the length, not gaps at the sequence ends.

As used herein, the term “a disease or disorder associated with an ARSA gene mutation” refers to any disease or disorder caused by, exacerbated by, or genetically linked with mutation of an ARSA gene. In certain embodiments, the disease or disorder associated with an ARSA gene mutation is metachromatic leukodystrophy (MLD).

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may either be wild-type or codon-altered. An exemplary wild-type ARSA coding sequence is set forth in SEQ ID NO:24.

As used herein, the term “silently altered” refers to alteration of a coding sequence or a stuffer-inserted coding sequence of a gene (e.g., by nucleotide substitution) without changing the amino acid sequence of the polypeptide encoded by the coding sequence or stuffer-inserted coding sequence. Such silent alteration is advantageous in that it may increase the translation efficiency of a coding sequence, and/or prevent recombination with a corresponding sequence of an endogenous gene when a coding sequence is transduced into a cell.

In the instant disclosure, nucleotide positions in an ARSA gene are specified relative to the first nucleotide of the start codon. The first nucleotide of a start codon is position 1; the nucleotides 5′ to the first nucleotide of the start codon have negative numbers; the nucleotides 3′ to the first nucleotide of the start codon have positive numbers. An exemplary nucleotide 1 of the human ARSA gene is nucleotide 374 of the NCBI Reference Sequence: NG 009260.2 (Region: 5028-10426), and an exemplary nucleotide 3 of the human ARSA gene is nucleotide 376 of the NCBI Reference Sequence: NG 009260.2 (Region: 5028-10426). The nucleotide adjacently 5′ to the start codon is nucleotide-1.

In the instant disclosure, exons and introns in an ARSA gene are specified relative to the exon encompassing the first nucleotide of the start codon, which is nucleotide 374 of the NCBI Reference Sequence: NG 009260.2 (Region: 5028-10426). The exon encompassing the first nucleotide of the start codon is exon 1. Exons 3′ to exon 1 are from 5′ to 3′: exon 2, exon 3, etc. Introns 3′ to exon 1 are from 5′ to 3′: intron 1, intron 2, etc. Accordingly, the ARSA gene comprises from 5′ to 3′: exon 1, intron 1, exon 2, intron 2, exon 3, etc. An exemplary exon 1 of the human ARSA gene is nucleotides 374-597 of the NCBI Reference Sequence: NG 009260.2 (Region: 5028-10426). An exemplary intron 1 of the human ARSA gene is nucleotides 598-746 of the NCBI Reference Sequence: NG 009260.2 (Region: 5028-10426).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer elements. The coding sequence is “operably linked” to the TRE if the transcription of the coding sequence is controlled or influenced by the TRE. The promoter and enhancer elements of the TRE may be in any orientation and/or distance from the coding sequence, as long as the desired transcriptional activity is obtained. In certain embodiments, the TRE is upstream from the coding sequence.

As used herein, the term “ribosomal skipping element” refers to a nucleotide sequence encoding a short peptide sequence capable of causing generation of two peptide chains from translation of one mRNA molecule. In certain embodiments, the ribosomal skipping element encodes a peptide comprising a consensus motif of X₁X₂EX₃NPGP, wherein X₁ is D or G, X₂ is V or I, and X₃ is any amino acid (SEQ ID NO: 34). In certain embodiments, the ribosomal skipping element encodes Thosea asigna virus 2A peptide (T2A), porcine teschovirus-1 2A peptide (P2A), foot-and-mouth disease virus 2A peptide (F2A), equine rhinitis A virus 2A peptide (E2A), cytoplasmic polyhedrosis virus 2A peptide (BmCPV 2A), or flacherie virus of B. mori 2A peptide (BmIFV 2A). Exemplary amino acid sequences of T2A peptide and P2A peptide are set forth in SEQ ID NO: 37 and 38, respectively. Exemplary nucleotide sequences of T2A element and P2A element are set forth in SEQ ID NO: 66 and 63, respectively. In certain embodiments, the ribosomal skipping element encodes a peptide that further comprises a sequence of Gly-Ser-Gly at the N terminus, optionally wherein the sequence of Gly-Ser-Gly is encoded by the nucleotide sequence of GGCAGCGGA. While not wishing to be bound by theory, it is hypothesized that ribosomal skipping elements function by: terminating translation of the first peptide chain and re-initiating translation of the second peptide chain; or by cleavage of a peptide bond in the peptide sequence encoded by the ribosomal skipping element by an intrinsic protease activity of the encoded peptide, or by another protease in the environment (e.g., cytosol).

As used herein, the term “ribosomal skipping peptide” refers to a peptide encoded by a ribosomal skipping element.

As used herein, the term “polyadenylation sequence” refers to a DNA sequence that when transcribed into RNA constitutes a polyadenylation signal sequence. The polyadenylation sequence can be native (e.g., from the ARSA gene) or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of an ARSA gene (e.g., human ARSA gene). In certain embodiments, an exogenous polyadenylation sequence is a polyadenylation sequence of a non-ARSA gene in the same species (e.g., human). In certain embodiments, an exogenous polyadenylation sequence is a polyadenylation sequence of a different species (e.g., a virus).

As used herein, the term “effective amount” in the context of the administration of an AAV to a subject refers to the amount of the AAV that achieves a desired prophylactic or therapeutic effect.

### II. ADENO-ASSOCIATED VIRUS COMPOSITIONS

In one aspect, provided herein are novel recombinant AAV (e.g., replication-defective AAV) compositions useful for expressing an ARSA polypeptide in cells with reduced or otherwise defective ARSA gene function. In certain embodiments, the rAAV disclosed herein comprise: an AAV capsid comprising a capsid protein (e.g., a Clade F capsid protein); and a transfer genome comprising a transcriptional regulatory element operably linked to an ARSA coding sequence (e.g., a silently altered ARSA coding sequence), allowing for extrachromosomal expression of ARSA in a cell transduced with the AAV.

A capsid protein from any capsid known the art can be used in the rAAV compositions disclosed herein, including, without limitation, a capsid protein from an AAV1, AAV2, AAV3, AAV4, AAV5, AAV6, AAV7, AAV8, or AAV9 serotype. For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 203-736 of SEQ ID NO: 2, 3, 4, 6, 7, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 138-736 of SEQ ID NO: 2, 3, 4, 5, 6, 7, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 2 of SEQ ID NO: 16 is T, and the amino acid in the capsid protein corresponding to amino acid 312 of SEQ ID NO: 16 is Q. In certain embodiments, the amino acid in the capsid protein corresponding to amino acid 65 of SEQ ID NO: 16 is I, and the amino acid in the capsid protein corresponding to amino acid 626 of SEQ ID NO: 16 is Y. In certain embodiments, the amino acid in the capsid protein corresponding to amino acid 77 of SEQ ID NO: 16 is R, and the amino acid in the capsid protein corresponding to amino acid 690 of SEQ ID NO: 16 is K. In certain embodiments, the amino acid in the capsid protein corresponding to amino acid 119 of SEQ ID NO: 16 is L, and the amino acid in the capsid protein corresponding to amino acid 468 of SEQ ID NO: 16 is S. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 1-736 of SEQ ID NO: 2, 3, 4, 5, 6, 7, 8, 9, 10, 11, 12, 13, 15, 16, or 17.

In certain embodiments, the AAV capsid comprises two or more of: (a) a capsid protein comprising the amino acid sequence of amino acids 203-736 of SEQ ID NO: 2, 3, 4, 6, 7, 10, 11, 12, 13, 15, 16, or 17; (b) a capsid protein comprising the amino acid sequence of amino acids 138-736 of SEQ ID NO: 2, 3, 4, 5, 6, 7, 9, 10, 11, 12, 13, 15, 16, or 17; and (c) a capsid protein comprising the amino acid sequence of amino acids 1-736 of SEQ ID NO: 2, 3, 4, 5, 6, 7, 8, 9, 10, 11, 12, 13, 15, 16, or 17. In certain embodiments, the AAV capsid comprises: (a) a capsid protein having an amino acid sequence consisting of amino acids 203-736 of SEQ ID NO: 2, 3, 4, 6, 7, 10, 11, 12, 13, 15, 16, or 17; (b) a capsid protein having an amino acid sequence consisting of amino acids 138-736 of SEQ ID NO: 2, 3, 4, 5, 6, 7, 9, 10, 11, 12, 13, 15, 16, or 17; and (c) a capsid protein having an amino acid sequence consisting of amino acids 1-736 of SEQ ID NO: 2, 3, 4, 5, 6, 7, 8, 9, 10, 11, 12, 13, 15, 16, or 17.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 8; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 8; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 8. In certain embodiments, the AAV capsid comprises one or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises two or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises: (a) a capsid protein having an amino acid sequence consisting of amino acids 203-736 of SEQ ID NO: 8; (b) a capsid protein having an amino acid sequence consisting of amino acids 138-736 of SEQ ID NO: 8; and (c) a capsid protein having an amino acid sequence consisting of amino acids 1-736 of SEQ ID NO: 8.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 11; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 11; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 11. In certain embodiments, the AAV capsid comprises one or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises two or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises: (a) a capsid protein having an amino acid sequence consisting of amino acids 203-736 of SEQ ID NO: 11; (b) a capsid protein having an amino acid sequence consisting of amino acids 138-736 of SEQ ID NO: 11; and (c) a capsid protein having an amino acid sequence consisting of amino acids 1-736 of SEQ ID NO: 11.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 13; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 13; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 13. In certain embodiments, the AAV capsid comprises one or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises two or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises: (a) a capsid protein having an amino acid sequence consisting of amino acids 203-736 of SEQ ID NO: 13; (b) a capsid protein having an amino acid sequence consisting of amino acids 138-736 of SEQ ID NO: 13; and (c) a capsid protein having an amino acid sequence consisting of amino acids 1-736 of SEQ ID NO: 13.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 16; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 16; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 16. In certain embodiments, the AAV capsid comprises one or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises two or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises: (a) a capsid protein having an amino acid sequence consisting of amino acids 203-736 of SEQ ID NO: 16; (b) a capsid protein having an amino acid sequence consisting of amino acids 138-736 of SEQ ID NO: 16; and (c) a capsid protein having an amino acid sequence consisting of amino acids 1-736 of SEQ ID NO: 16.

Transfer genomes useful in the AAV compositions disclosed herein generally comprise a transcriptional regulatory element (TRE) operably linked to an ARSA coding sequence. In certain embodiments, the transfer genome comprises a 5′ inverted terminal repeat (5′ ITR) nucleotide sequence 5′ of the TRE and ARSA coding sequence, and a 3′ inverted terminal repeat (3′ ITR) nucleotide sequence 3′ of the TRE and ARSA coding sequence.

In certain embodiments, the ARSA coding sequence comprises all or substantially all of a coding sequence of an ARSA gene. In certain embodiments, the transfer genome comprises a nucleotide sequence encoding SEQ ID NO: 23 and can optionally further comprise an exogenous polyadenylation sequence 3′ to the ARSA coding sequence. In certain embodiments, the nucleotide sequence encoding SEQ ID NO: 23 is wild-type (e.g., having the sequence set forth in SEQ ID NO: 24). In certain embodiments, the nucleotide sequence encoding SEQ ID NO: 23 is silently-altered (e.g., having the sequence set forth in SEQ ID NO: 14, 62, or 72).

In certain embodiments, the ARSA coding sequence encodes a polypeptide comprising all or substantially all of the amino acids sequence of an ARSA protein. In certain embodiments, the ARSA coding sequence encodes the amino acid sequence of a wild-type ARSA protein (e.g., human ARSA protein). In certain embodiments, the ARSA coding sequence encodes the amino acid sequence of a mutant ARSA protein (e.g., human ARSA protein), wherein the mutant ARSA polypeptide is a functional equivalent of the wild-type ARSA polypeptide, i.e., can function as a wild-type ARSA polypeptide. In certain embodiments, the functionally equivalent ARSA polypeptide further comprises at least one characteristic not found in the wild-type ARSA polypeptide, e.g., the ability to resist protein degradation.

In certain embodiments, transfer genomes useful in the AAV compositions disclosed herein generally comprise a transcriptional regulatory element (TRE) operably linked to a coding sequence encoding for ARSA and/or SUMF1. The sulfatase modifying factor 1 (SUMF1) gene encodes an enzyme that catalyzes the hydrolysis of sulfate esters by oxidizing a cysteine residue in the substrate sulfatase to an active site 3-oxoalanine residue, which is also known as C-alpha-formylglycine. Diseases associated with SUMF1 include multiple sulfatase deficiency and metachromatic leukodystrophy.

In certain embodiments, the SUMF1 coding sequence comprises all or substantially all of a coding sequence of a SUMF1 gene. In certain embodiments, the transfer genome comprises a nucleotide sequence encoding SEQ ID NO: 29 and can optionally further comprise an exogenous polyadenylation sequence 3′ to the SUMF1 coding sequence. In certain embodiments, the nucleotide sequence encoding SEQ ID NO: 29 is wild-type (e.g., having the sequence set forth in SEQ ID NO: 64). In certain embodiments, the nucleotide sequence encoding SEQ ID NO: 29 is silently-altered.

In certain embodiments, the SUMF1 coding sequence encodes a polypeptide comprising all or substantially all of the amino acids sequence of an SUMF1 protein. In certain embodiments, the SUMF1 coding sequence encodes the amino acid sequence of a wild-type SUMF1 protein (e.g., human SUMF1 protein (hSUMF1)). In certain embodiments, the SUMF1 coding sequence encodes the amino acid sequence of a mutant SUMF1 protein (e.g., human SUMF1 protein), wherein the mutant SUMF1 polypeptide is a functional equivalent of the wild-type SUMF1 polypeptide, i.e., can function as a wild-type SUMF1 polypeptide. In certain embodiments, the functionally equivalent SUMF1 polypeptide further comprises at least one characteristic not found in the wild-type SUMF1 polypeptide, e.g., the ability to resist protein degradation.

In certain embodiments, the transfer genome is designed to express both hARSA and hSUMF1, and comprises a nucleotide sequence that comprises a first coding sequence encoding for hARSA, and a second coding sequence encoding for hSUMF1. In certain embodiments, the first coding sequence encoding for hARSA and the second coding sequence encoding for hSUMF1 is separated by a ribosomal skipping element. Any ribosomal skipping element known in the art may be used, for example, the ribosomal skipping elements described elsewhere herein. In certain embodiments, the nucleotide sequence that comprises a first coding sequence encoding for hARSA and a second coding sequence encoding for hSUMF1 comprises the nucleotide sequence set forth in SEQ ID NO: 30.

In certain embodiments, transfer genomes useful in the AAV compositions disclosed herein generally comprise a transcriptional regulatory element (TRE) operably linked to a coding sequence encoding for ARSA and/or SapB. The Prosaposin (PSAP) gene encodes a highly conserved preproprotein that is proteolytically processed to generate four main cleavage products including saposins A, B, C, and D. Each domain of the precursor protein is approximately 80 amino acid residues long with nearly identical placement of cysteine residues and glycosylation sites. Saposins A-D localize primarily to the lysosomal compartment where they facilitate the catabolism of glycosphingolipids with short oligosaccharide groups. The precursor protein exists both as a secretory protein and as an integral membrane protein and has neurotrophic activities. Mutations in this gene have been associated with Gaucher disease and metachromatic leukodystrophy. Saposin B (SapB) has been shown to stimulate the hydrolysis of galacto-cerebroside sulfate by ARSA, GM1 gangliosides by beta-galactosidase, and globotriaosylceramide by alpha-galactosidase A. SapB has been shown to form a solubilizing complex with the substrates of the sphingolipid hydrolases.

In certain embodiments, the SapB coding sequence comprises all or substantially all of a coding sequence of a SapB gene. In certain embodiments, the transfer genome comprises a nucleotide sequence encoding SEQ ID NO: 33 and can optionally further comprise an exogenous polyadenylation sequence 3′ to the SapB coding sequence. In certain embodiments, the nucleotide sequence encoding SEQ ID NO: 33 is wild-type (e.g., having the sequence set forth in SEQ ID NO: 73). In certain embodiments, the nucleotide sequence encoding SEQ ID NO: 33 is silently-altered.

In certain embodiments, the SapB coding sequence encodes a polypeptide comprising all or substantially all of the amino acids sequence of an SapB protein. In certain embodiments, the SapB coding sequence encodes the amino acid sequence of a wild-type SapB protein (e.g., human SapB protein (hSapB)). In certain embodiments, the SapB coding sequence encodes the amino acid sequence of a mutant SapB protein (e.g., human SapB protein), wherein the mutant SapB polypeptide is a functional equivalent of the wild-type SapB polypeptide, i.e., can function as a wild-type SapB polypeptide. In certain embodiments, the functionally equivalent SapB polypeptide further comprises at least one characteristic not found in the wild-type SapB polypeptide, e.g., the ability to resist protein degradation.

In certain embodiments, the transfer genome is designed to express both hARSA and hSapB, and comprises a nucleotide sequence that comprises a first coding sequence encoding for hARSA, and a second coding sequence encoding for hSapB. In certain embodiments, the first coding sequence encoding for hARSA and the second coding sequence encoding for hSapB is separated by a ribosomal skipping element. Any ribosomal skipping element known in the art may be used, for example, the ribosomal skipping elements described elsewhere herein. In certain embodiments, the nucleotide sequence that comprises a first coding sequence encoding for hARSA and a second coding sequence encoding for hSapB comprises the nucleotide sequence set forth in SEQ ID NO: 74.

The transfer genome can be used to express ARSA, SUMF1, and/or SapB in any mammalian cells (e.g., human cells). Thus, the TRE can be active in any mammalian cells (e.g., human cells). In certain embodiments, the TRE is active in a broad range of human cells. Such TREs may comprise constitutive promoter and/or enhancer elements including cytomegalovirus (CMV) promoter/enhancer (e.g., comprising a nucleotide sequence at least 90%, 91%, 92%, 93%, 94%, 95%, 96%, 97%, 98%, 99%, or 100% identical to SEQ ID NO: 58), SV40 promoter, chicken beta actin (CBA) promoter (e.g., comprising a nucleotide sequence at least 90%, 91%, 92%, 93%, 94%, 95%, 96%, 97%, 98%, 99%, or 100% identical to SEQ ID NO: 59 or 25), smCBA promoter (e.g., comprising a nucleotide sequence at least 90%, 91%, 92%, 93%, 94%, 95%, 96%, 97%, 98%, 99%, or 100% identical to SEQ ID NO: 55), human elongation factor 1 alpha (EF1α) promoter (e.g., comprising a nucleotide sequence at least 90%, 91%, 92%, 93%, 94%, 95%, 96%, 97%, 98%, 99%, or 100% identical to SEQ ID NO: 40), minute virus of mouse (MVM) intron which comprises transcription factor binding sites (e.g., comprising a nucleotide sequence at least 90%, 91%, 92%, 93%, 94%, 95%, 96%, 97%, 98%, 99%, or 100% identical to SEQ ID NO: 35), human phosphoglycerate kinase (PGK1) promoter, human ubiquitin C (Ubc) promoter, human beta actin promoter, human neuron-specific enolase (ENO2) promoter, human beta-glucuronidase (GUSB) promoter, a rabbit beta-globin element (e.g., comprising a nucleotide sequence at least 90%, 91%, 92%, 93%, 94%, 95%, 96%, 97%, 98%, 99%, or 100% identical to SEQ ID NO: 60), human calmodulin 1 (CALM1) promoter (e.g., comprising a nucleotide sequence at least 90%, 91%, 92%, 93%, 94%, 95%, 96%, 97%, 98%, 99%, or 100% identical to SEQ ID NO: 54), and/or human Methyl-CpG Binding Protein 2 (MeCP2) promoter. Any of these TREs can be combined in any order to drive efficient transcription. For example, a transfer genome may comprise a CMV enhancer, a CBA promoter, and the splice acceptor from exon 3 of the rabbit beta-globin gene, collectively called a CAG promoter (e.g., comprising a nucleotide sequence at least 90%, 91%, 92%, 93%, 94%, 95%, 96%, 97%, 98%, 99%, or 100% identical to SEQ ID NO: 28). For example, a transfer genome may comprise a hybrid of CMV enhancer and CBA promoter followed by a splice donor and splice acceptor, collectively called a CASI promoter region (e.g., comprising a nucleotide sequence at least 90%, 91%, 92%, 93%, 94%, 95%, 96%, 97%, 98%, 99%, or 100% identical to SEQ ID NO: 63).

Alternatively, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, which is incorporated by reference herein in its entirety.

In certain embodiments, the transfer genome comprises two or more TREs, optionally comprising at least one of the TREs disclosed above. A skilled person in the art would appreciate that any of these TREs can be combined in any order, and combinations of a constitutive TRE and a tissue-specific TRE can drive efficient and tissue-specific transcription.

In certain embodiments, the transfer vector further comprises a non-coding stuffer sequence (e.g., comprising a nucleotide sequence at least 90%, 91%, 92%, 93%, 94%, 95%, 96%, 97%, 98%, 99%, or 100% identical to SEQ ID NO: 39). Non-coding stuffer sequences may be employed to maintain the size of a vector within appropriate limits for efficient DNA packaging, and as such may be employed to increase the efficacy of DNA packaging. Those of skill in the art will recognize that the nature of the stuffer sequence may have an effect on the function of the vector, and will accordingly, select the most suitable stuffer sequence for use.

In certain embodiments, the transfer vector further comprises an intron 5′ to or inserted in the ARSA coding sequence. Such introns can increase transgene expression, for example, by reducing transcriptional silencing and enhancing mRNA export from the nucleus to the cytoplasm. In certain embodiments, the transfer genome comprises from 5′ to 3′: a non-coding exon, an intron, and the ARSA coding sequence. In certain embodiments, an intron sequence is inserted in the ARSA coding sequence, optionally wherein the intron is inserted at an internucleotide bond that links two native exons. In certain embodiments, the intron is inserted at an internucleotide bond that links native exon 1 and exon 2.

The intron can comprise a native intron sequence of the ARSA gene, an intron sequence from a different species or a different gene from the same species, and/or a synthetic intron sequence. A skilled worker will appreciate that synthetic intron sequences can be designed to mediate RNA splicing by introducing any consensus splicing motifs known in the art (e.g., in Sibley et al., (2016) Nature Reviews Genetics, 17, 407-21, which is incorporated by reference herein in its entirety). Exemplary intron sequences are provided in Lu et al. (2013) Molecular Therapy 21(5): 954-63, and Lu et al. (2017) Hum. Gene Ther. 28(1): 125-34, which are incorporated by reference herein in their entirety. In certain embodiments, the transfer genome comprises an SV40 intron (e.g., comprising a nucleotide sequence at least 90%, 91%, 92%, 93%, 94%, 95%, 96%, 97%, 98%, or 99% identical to SEQ ID NO: 31) or a minute virus of mouse (MVM) intron (e.g., comprising a nucleotide sequence at least 90%, 91%, 92%, 93%, 94%, 95%, 96%, 97%, 98%, or 99% identical to SEQ ID NO: 35). In certain embodiments, the transfer genome comprises an SV40 intron (e.g., comprising the nucleotide sequence set forth in SEQ ID NO: 31) or a minute virus of mouse (MVM) intron (e.g., comprising the nucleotide sequence set forth in SEQ ID NO: 35). In certain embodiments, the transfer genome comprises a chimeric intron sequence comprising a combination of chicken and rabbit sequences, comprising partially the untranscribed chicken ACTB (cACTB) promoter, all of cACTB exon 1, partially cACTB intron 1, partially rabbit HBB2 (rHBB2) intron 2, and partially rHBB2 exon 3 (e.g., SEQ ID NO: 32). In certain embodiments, the transfer genome comprises a chimeric intron sequence (e.g., comprising a nucleotide sequence at least 90%, 91%, 92%, 93%, 94%, 95%, 96%, 97%, 98%, or 99% identical to SEQ ID NO: 32). In certain embodiments, the transfer genome comprises a chimeric intron sequence (e.g., comprising the nucleotide sequence set forth in SEQ ID NO: 32).

In certain embodiments, the transfer genome comprises a TRE comprising a CMV enhancer, a CBA promoter, and a chimeric intron sequence (e.g., comprising a nucleotide sequence at least 90%, 91%, 92%, 93%, 94%, 95%, 96%, 97%, 98%, or 99% identical to SEQ ID NO: 36). In certain embodiments, the transfer genome comprises a TRE comprising SEQ ID NO: 36.

In certain embodiments, the transfer genome disclosed herein further comprises a transcription terminator (e.g., a polyadenylation sequence). In certain embodiments, the transcription terminator is 3′ to the ARSA coding sequence. The transcription terminator may be any sequence that effectively terminates transcription, and a skilled artisan would appreciate that such sequences can be isolated from any genes that are expressed in the cell in which transcription of the ARSA coding sequence is desired. In certain embodiments, the transcription terminator comprises a polyadenylation sequence. In certain embodiments, the polyadenylation sequence is identical or substantially identical to the endogenous polyadenylation sequence of the human ARSA gene. In certain embodiments, the polyadenylation sequence is an exogenous polyadenylation sequence. In certain embodiments, the polyadenylation sequence is an SV40 polyadenylation sequence (e.g., comprising the nucleotide sequence set forth in SEQ ID NO: 31, 42, 43, or 45, or a nucleotide sequence complementary thereto). In certain embodiments, the polyadenylation sequence comprises the sequence set forth in SEQ ID NO: 42.

In certain embodiments, the transfer genome comprises from 5′ to 3′: a TRE, an ARSA coding sequence, and a polyadenylation sequence. In certain embodiments, the TRE has at least 90%, 91%, 92%, 93%, 94%, 95%, 96%, 97%, 98%, or 99% sequence identity to any one of SEQ ID NO: 25, 32, 36, 54, 55, and/or 58; the ARSA coding sequence has at least 90%, 91%, 92%, 93%, 94%, 95%, 96%, 97%, 98%, or 99% sequence identity to SEQ ID NO: 14, 24, 62, or 72; and/or the polyadenylation sequence has at least 90%, 91%, 92%, 93%, 94%, 95%, 96%, 97%, 98%, or 99% sequence identity to any one of SEQ ID NO: 42, 43, and 45.

In certain embodiments, the TRE comprises the sequence set forth in SEQ ID NO: 36; the ARSA coding sequence comprises the sequence set forth in SEQ ID NO: 14; and/or the polyadenylation sequence comprises the sequence set forth in SEQ ID NO: 42. In certain embodiments, the TRE comprises from 5′ to 3′ the sequence set forth in SEQ ID NO: 58, the sequence set forth in SEQ ID NO: 25, and the sequence set forth in SEQ ID NO: 32.

In certain embodiments, the TRE comprises the sequence set forth in SEQ ID NO: 54; the ARSA coding sequence comprises the sequence set forth in SEQ ID NO: 62; and/or the polyadenylation sequence comprises the sequence set forth in SEQ ID NO: 42. In certain embodiments, the TRE comprises the sequence set forth in SEQ ID NO: 55; the ARSA coding sequence comprises the sequence set forth in SEQ ID NO: 62; and/or the polyadenylation sequence comprises the sequence set forth in SEQ ID NO: 42.

In certain embodiments, the TRE comprises the sequence set forth in SEQ ID NO: 36; the ARSA coding sequence comprises the sequence set forth in SEQ ID NO: 72; and/or the polyadenylation sequence comprises the sequence set forth in SEQ ID NO: 42. In certain embodiments, the TRE comprises from 5′ to 3′ the sequence set forth in SEQ ID NO: 58, the sequence set forth in SEQ ID NO: 25, and the sequence set forth in SEQ ID NO: 32.

In certain embodiments, the transfer genome further comprises a hSUMF1 coding sequence. In certain embodiments, the transfer genome comprises from 5′ to 3′: a TRE, an ARSA coding sequence, a 2A element, and a hSUMF1 coding sequence. In certain embodiments, the TRE has at least 90%, 91%, 92%, 93%, 94%, 95%, 96%, 97%, 98%, or 99% sequence identity to SEQ ID NO: 25, 32, 36, 54, 55, and/or 58; the ARSA coding sequence has at least 90%, 91%, 92%, 93%, 94%, 95%, 96%, 97%, 98%, or 99% sequence identity to SEQ ID NO: 62; the 2A element has at least 90%, 91%, 92%, 93%, 94%, 95%, 96%, 97%, 98%, or 99% sequence identity to SEQ ID NO: 63; and the hSUMF1 sequence has at least 90%, 91%, 92%, 93%, 94%, 95%, 96%, 97%, 98%, or 99% sequence identity to SEQ ID NO: 64. In certain embodiments, a transfer genome that further comprises a hSUMF1 coding sequence comprises from 5′ to 3′: a TRE comprising the sequence set forth in SEQ ID NO: 54 or 55, a hARSA coding sequence comprising the sequence set forth in SEQ ID NO: 62, a 2A element comprising the sequence set forth in SEQ ID NO: 63, and a hSUMF1 coding sequence comprising the sequence set forth in SEQ ID NO: 64. In certain embodiments, the hARSA-2A-hSUMF1 coding sequence comprises the sequence set forth in SEQ ID NO: 30.

In certain embodiments, the transfer genome further comprises a hSapB coding sequence. In certain embodiments, the transfer genome comprises from 5′ to 3′: a TRE, an ARSA coding sequence, a 2A element, and a hSapB coding sequence. In certain embodiments, the TRE has at least 90%, 91%, 92%, 93%, 94%, 95%, 96%, 97%, 98%, or 99% sequence identity to SEQ ID NO: 25, 32, 36, 54, 55, and/or 58; the ARSA coding sequence has at least 90%, 91%, 92%, 93%, 94%, 95%, 96%, 97%, 98%, or 99% sequence identity to SEQ ID NO: 72; the 2A element has at least 90%, 91%, 92%, 93%, 94%, 95%, 96%, 97%, 98%, or 99% sequence identity to SEQ ID NO: 63; and the hSapB sequence has at least 90%, 91%, 92%, 93%, 94%, 95%, 96%, 97%, 98%, or 99% sequence identity to SEQ ID NO: 73. In certain embodiments, a transfer genome that further comprises a hSapB coding sequence comprises from 5′ to 3′: a TRE comprising the sequence set forth in SEQ ID NO: 36, a hARSA coding sequence comprising the sequence set forth in SEQ ID NO: 72, a 2A element comprising the sequence set forth in SEQ ID NO: 63, and a hSapB coding sequence comprising the sequence set forth in SEQ ID NO: 74. In certain embodiments, the hARSA-2A-hSapB coding sequence comprises the sequence set forth in SEQ ID NO: 74.

In certain embodiments, the transfer genome comprises a sequence at least 80%, 85%, 90%, 91%, 92%, 93%, 94%, 95%, 96%, 97%, 98%, or 99% identical to SEQ ID NO: 41, 44, 46, 65, 67, or 75. In certain embodiments, the transfer genome comprises the nucleotide sequence set forth in SEQ ID NO: 41, 44, 46, 65, 67, or 75. In certain embodiments, the nucleotide sequence of the transfer genome consists of the nucleotide sequence set forth in SEQ ID NO: 41, 44, 46, 65, 67, or 75. In certain embodiments, the transfer genome comprises the nucleotide sequence set forth in SEQ ID NO: 44. In certain embodiments, the nucleotide sequence of the transfer genome consists of the nucleotide sequence set forth in SEQ ID NO: 44.

In certain embodiments, the transfer genomes disclosed herein further comprise a 5′ inverted terminal repeat (5′ ITR) nucleotide sequence 5′ of the TRE, and a 3′ inverted terminal repeat (3′ ITR) nucleotide sequence 3′ of the ARSA coding sequence. ITR sequences from any AAV serotype or variant thereof can be used in the transfer genomes disclosed herein. The 5′ and 3′ ITR can be from an AAV of the same serotype or from AAVs of different serotypes. Exemplary ITRs for use in the transfer genomes disclosed herein are set forth in SEQ ID NO: 18-21, 26, and 27 herein.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 90% (e.g., at least 95%, at least 96%, at least 97%, at least 98%, at least 99%, or 100%) sequence identity to SEQ ID NO: 18, or the 3′ ITR nucleotide sequence has at least 90% (e.g., at least 95%, at least 96%, at least 97%, at least 98%, at least 99%, or 100%) sequence identity to SEQ ID NO: 19. In certain embodiments, the 5′ ITR nucleotide sequence has at least 90% (e.g., at least 95%, at least 96%, at least 97%, at least 98%, at least 99%, or 100%) sequence identity to SEQ ID NO: 18, and the 3′ ITR nucleotide sequence has at least 90% (e.g., at least 95%, at least 96%, at least 97%, at least 98%, at least 99%, or 100%) sequence identity to SEQ ID NO: 19. In certain embodiments, the transfer genome comprises a nucleotide sequence set forth in any one of SEQ ID NO: 41, 44, 46, 65, 67, or 75, a 5′ ITR nucleotide sequence having the sequence of SEQ ID NO: 18, and a 3′ ITR nucleotide sequence having the sequence of SEQ ID NO: 19.

In certain embodiments, the 5′ ITR or 3′ ITR are from AAV5. In certain embodiments, both the 5′ ITR and 3′ ITR are from AAV5. In certain embodiments, the 5′ ITR nucleotide sequence has at least 90% (e.g., at least 95%, at least 96%, at least 97%, at least 98%, at least 99%, or 100%) sequence identity to SEQ ID NO: 20, or the 3′ ITR nucleotide sequence has at least 90% (e.g., at least 95%, at least 96%, at least 97%, at least 98%, at least 99%, or 100%) sequence identity to SEQ ID NO: 21. In certain embodiments, the 5′ ITR nucleotide sequence has at least 90% (e.g., at least 95%, at least 96%, at least 97%, at least 98%, at least 99%, or 100%) sequence identity to SEQ ID NO: 20, and the 3′ ITR nucleotide sequence has at least 90% (e.g., at least 95%, at least 96%, at least 97%, at least 98%, at least 99%, or 100%) sequence identity to SEQ ID NO: 21. In certain embodiments, the transfer genome comprises a nucleotide sequence set forth in any one of SEQ ID NO: 46-50, a 5′ ITR nucleotide sequence having the sequence of SEQ ID NO: 20, and a 3′ ITR nucleotide sequence having the sequence of SEQ ID NO: 21.

In certain embodiments, the 5′ ITR nucleotide sequence and the 3′ ITR nucleotide sequence are substantially complementary to each other (e.g., are complementary to each other except for mismatch at 1, 2, 3, 4, or 5 nucleotide positions in the 5′ or 3′ ITR).

In certain embodiments, the 5′ ITR or the 3′ ITR is modified to reduce or abolish resolution by Rep protein (“non-resolvable ITR”). In certain embodiments, the non-resolvable ITR comprises an insertion, deletion, or substitution in the nucleotide sequence of the terminal resolution site. Such modification allows formation of a self-complementary, double-stranded DNA genome of the AAV after the transfer genome is replicated in an infected cell. Exemplary non-resolvable ITR sequences are known in the art (see e.g., those provided in U.S. Pat. Nos. 7,790,154 and 9,783,824, which are incorporated by reference herein in their entirety). In certain embodiments, the 5′ ITR comprises a nucleotide sequence at least 95%, 96%, 97%, 98%, or 99% identical to SEQ ID NO: 26. In certain embodiments, the 5′ ITR consists of a nucleotide sequence at least 95%, 96%, 97%, 98%, or 99% identical to SEQ ID NO: 26. In certain embodiments, the 5′ ITR consists of the nucleotide sequence set forth in SEQ ID NO: 26. In certain embodiments, the 3′ ITR comprises a nucleotide sequence at least 95%, 96%, 97%, 98%, or 99% identical to SEQ ID NO: 27. In certain embodiments, the 5′ ITR consists of a nucleotide sequence at least 95%, 96%, 97%, 98%, or 99% identical to SEQ ID NO: 27. In certain embodiments, the 3′ ITR consists of the nucleotide sequence set forth in SEQ ID NO: 27. In certain embodiments, the 5′ ITR consists of the nucleotide sequence set forth in SEQ ID NO: 26, and the 3′ ITR consists of the nucleotide sequence set forth in SEQ ID NO: 27. In certain embodiments, the 5′ ITR consists of the nucleotide sequence set forth in SEQ ID NO: 26, and the 3′ ITR consists of the nucleotide sequence set forth in SEQ ID NO: 19.

In certain embodiments, the 3′ ITR is flanked by an additional nucleotide sequence derived from a wild-type AAV2 genomic sequence. In certain embodiments, the 3′ ITR is flanked by an additional 37 bp sequence derived from a wild-type AAV2 sequence that is adjacent to a wild-type AAV2 ITR. See, e.g., Savy et al., Human Gene Therapy Methods (2017) 28(5): 277-289 (which is hereby incorporated by reference herein in its entirety). In certain embodiments, the additional 37 bp sequence is internal to the 3′ ITR. In certain embodiments, the 37 bp sequence consists of the sequence set forth in SEQ ID NO: 56. In certain embodiments, the 3′ ITR comprises a nucleotide sequence at least 95%, 96%, 97%, 98%, or 99% identical to SEQ ID NO: 57. In certain embodiments, the 3′ ITR comprises the nucleotide sequence set forth in SEQ ID NO: 57. In certain embodiments, the nucleotide sequence of the 3′ ITR consists of a nucleotide sequence at least 95%, 96%, 97%, 98%, or 99% identical to SEQ ID NO: 57. In certain embodiments, the nucleotide sequence of the 3′ ITR consists of the nucleotide sequence set forth in SEQ ID NO: 57.

In certain embodiments, the transfer genome comprises from 5′ to 3′: a 5′ ITR; an internal element comprising from 5′ to 3′: a TRE, optionally a non-coding exon and an intron, an ARSA coding sequence, and a polyadenylation sequence, as disclosed herein; a non-resolvable ITR; a nucleotide sequence complementary to the internal element; and a 3′ ITR. Such transfer genome can form a self-complementary, double-stranded DNA genome of the AAV after infection and before replication.

In certain embodiments, the transfer genome comprises from 5′ to 3′: a 5′ ITR, a TRE, an ARSA coding sequence, a polyadenylation sequence, and a 3′ ITR. In certain embodiments, the 5′ ITR has at least 90%, 91%, 92%, 93%, 94%, 95%, 96%, 97%, 98%, or 99% sequence identity to SEQ ID: 18, 20, or 26; the TRE has at least 90%, 91%, 92%, 93%, 94%, 95%, 96%, 97%, 98%, or 99% sequence identity to any one of SEQ ID NO: 25, 32, 36, 54, 55, and/or 58; the ARSA coding sequence has at least 90%, 91%, 92%, 93%, 94%, 95%, 96%, 97%, 98%, or 99% sequence identity to SEQ ID NO: 14, 24, 62, or 72; the polyadenylation sequence has at least 90%, 91%, 92%, 93%, 94%, 95%, 96%, 97%, 98%, or 99% sequence identity to any one of SEQ ID NO: 42, 43, and 45; and/or the 3′ ITR has at least 90%, 91%, 92%, 93%, 94%, 95%, 96%, 97%, 98%, or 99% sequence identity to SEQ ID: 19, 21, 27, or 57. In certain embodiments, the 5′ ITR comprises or consists of a nucleotide sequence selected from the group consisting of SEQ ID NO: 18, 20, and 26; the TRE comprises a nucleotide sequence selected from the group consisting of SEQ ID NO: 25, 32, 36, 54, 55, and/or 58; the ARSA coding sequence comprises the sequence set forth in SEQ ID NO: 14, 24, 62, or 72; the polyadenylation sequence comprises a nucleotide sequence selected from the group consisting of SEQ ID NO: 42, 43, and 45; and/or the 3′ ITR comprises or consists of a nucleotide sequence selected from the group consisting of SEQ ID NO: 19, 21, 27, or 57.

In certain embodiments, the 5′ ITR comprises or consists of the sequence set forth in SEQ ID NO: 18; the TRE comprises the sequence set forth in SEQ ID NO: 36; the ARSA coding sequence comprises the sequence set forth in SEQ ID NO: 14, 24, 62, or 72; the polyadenylation sequence comprises the sequence set forth in SEQ ID NO: 42; and/or the 3′ ITR comprises or consists of the sequence set forth in SEQ ID NO: 19.

In certain embodiments, the transfer genome comprises a sequence at least 80%, 85%, 90%, 91%, 92%, 93%, 94%, 95%, 96%, 97%, 98%, or 99% identical to SEQ ID NO: 47, 48, 49, 68, 69, or 76. In certain embodiments, the transfer genome comprises the nucleotide sequence set forth in SEQ ID NO: 47, 48, 49, 68, 69, or 76. In certain embodiments, the nucleotide sequence of the transfer genome consists of the nucleotide sequence set forth in SEQ ID NO: 47, 48, 49, 68, 69, or 76. In certain embodiments, the transfer genome comprises the nucleotide sequence set forth in SEQ ID NO: 48. In certain embodiments, the nucleotide sequence of the transfer genome consists of the nucleotide sequence set forth in SEQ ID NO:

48.

In certain embodiments, the rAAV comprises: (a) an AAV capsid protein comprising the amino acid sequence of amino acids 203-736 of SEQ ID NO: 16, and a transfer genome comprising 5′ to 3′ following genetic elements: a 5′ ITR element (e.g., the 5′ ITR of SEQ ID NO: 18), an enhancer element (e.g., the enhancer element of SEQ ID NO: 58), a promoter sequence (e.g., the promoter sequence of SEQ ID NO: 25), a chimeric intron sequence (e.g., the chimeric intron sequence of SEQ ID NO: 32), a silently altered human ARSA coding sequence (e.g., the hARSA coding sequence of SEQ ID NO: 14), an SV40 polyadenylation sequence (e.g., the SV40 polyadenylation sequence of SEQ ID NO: 42), and a 3′ ITR element (e.g., the 3′ ITR of SEQ ID NO: 19); (b) an AAV capsid protein comprising the amino acid sequence of amino acids 138-736 of SEQ ID NO: 16, and a transfer genome comprising 5′ to 3′ following genetic elements: a 5′ ITR element (e.g., the 5′ ITR of SEQ ID NO: 18), an enhancer element (e.g., the enhancer element of SEQ ID NO: 58), a promoter sequence (e.g., the promoter sequence of SEQ ID NO: 25), a chimeric intron sequence (e.g., the chimeric intron sequence of SEQ ID NO: 32), a silently altered human ARSA coding sequence (e.g., the hARSA coding sequence of SEQ ID NO: 14), an SV40 polyadenylation sequence (e.g., the SV40 polyadenylation sequence of SEQ ID NO: 42), and a 3′ ITR element (e.g., the 3′ ITR of SEQ ID NO: 19); and/or (c) an AAV capsid protein comprising the amino acid sequence of SEQ ID NO: 16, and a transfer genome comprising 5′ to 3′ following genetic elements: a 5′ ITR element (e.g., the 5′ ITR of SEQ ID NO: 18), an enhancer element (e.g., the enhancer element of SEQ ID NO: 58), a promoter sequence (e.g., the promoter sequence of SEQ ID NO: 25), a chimeric intron sequence (e.g., the chimeric intron sequence of SEQ ID NO: 32), a silently altered human ARSA coding sequence (e.g., the hARSA coding sequence of SEQ ID NO: 14), an SV40 polyadenylation sequence (e.g., the SV40 polyadenylation sequence of SEQ ID NO: 42), and a 3′ ITR element (e.g., the 3′ ITR of SEQ ID NO: 19).

In certain embodiments, the rAAV comprises: (a) an AAV capsid protein comprising the amino acid sequence of amino acids 203-736 of SEQ ID NO: 16, and a transfer genome comprising the nucleotide sequence set forth in any one of SEQ ID NO: 41, 44, 46, 47, 48, 49, 65, 67, 68, 69, 75, or 76; (b) an AAV capsid protein comprising the amino acid sequence of amino acids 138-736 of SEQ ID NO: 16, and a transfer genome comprising the nucleotide sequence set forth in any one of SEQ ID NO: 41, 44, 46, 47, 48, 49, 65, 67, 68, 69, 75, or 76; and/or (c) an AAV capsid protein comprising the amino acid sequence of SEQ ID NO: 16, and a transfer genome comprising the nucleotide sequence set forth in any one of SEQ ID NO: 41, 44, 46, 47, 48, 49, 65, 67, 68, 69, 75, or 76.

In another aspect, provided herein is a polynucleotide comprising a nucleic acid sequence that is at least 80% (e.g., at least 85%, 90%, 91%, 92%, 93%, 94%, 95%, 96%, 97%, 98%, or 99%) identical to the nucleic acid sequence set forth in SEQ ID NO: 41, 44, 46, 47, 48, 49, 65, 67, 68, 69, 75, or 76. In certain embodiments, the polynucleotide comprises the nucleic acid sequence set forth in SEQ ID NO: 41, 44, 46, 47, 48, 49, 65, 67, 68, 69, 75, or 76. In certain embodiments, the polynucleotide consists of the nucleic acid sequence set forth in SEQ ID NO: 41, 44, 46, 47, 48, 49, 65, 67, 68, 69, 75, or 76. In certain embodiments, the polynucleotide comprises the nucleic acid sequence set forth in SEQ ID NO: 44 or 48. In certain embodiments, the polynucleotide consists of the nucleic acid sequence set forth in SEQ ID NO: 44 or 48.

Also provided herein is a polynucleotide comprising a nucleic acid sequence that is at least 80% (e.g., at least 85%, 90%, 91%, 92%, 93%, 94%, 95%, 96%, 97%, 98%, or 99%) identical to the nucleic acid sequence set forth in SEQ ID NO: 14, 62, or 72. In certain embodiments, the polynucleotide comprises the nucleic acid sequence set forth in SEQ ID NO: 14, 62, or 72. In certain embodiments, the polynucleotide consists of the nucleic acid sequence set forth in SEQ ID NO: 14, 62, or 72. In certain embodiments, the polynucleotide comprises the nucleic acid sequence set forth in SEQ ID NO: 14. In certain embodiments, the polynucleotide consists of the nucleic acid sequence set forth in SEQ ID NO: 14.

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al, 7th ed., Lippincott, Williams, & Wilkins; and Handbook of Pharmaceutical Excipients (2000) A. H. Kibbe et al, 3rd ed. Amer. Pharmaceutical Assoc.

In another aspect, the instant disclosure provides a polynucleotide comprising a coding sequence encoding a human ARSA protein or a fragment thereof, wherein the coding sequence has been silently-altered to have less than 100% (e.g., less than 95%, 90%, 85%, 80%, 75%, 70%, 65%, 60%, 55%, or 50%) identical to a wild-type human ARSA gene. In certain embodiments, the polynucleotide comprises the sequence set forth in SEQ ID NO: 14, 62, or 72. In certain embodiments, the polynucleotide consists of the sequence set forth in SEQ ID NO: 14, 62, or 72. The polynucleotide can comprise DNA, RNA, modified DNA, modified RNA, or a combination thereof. In certain embodiments, the polynucleotide is an expression vector.

### III. METHODS OF USE

In another aspect, the instant disclosure provides methods for expressing an ARSA polypeptide in a cell. The methods generally comprise transducing the cell with a rAAV as disclosed herein. Such methods are highly efficient at restoring ARSA expression. Accordingly, in certain embodiments, the methods disclosed herein involve transducing the cell with a rAAV as disclosed herein.

The methods disclosed herein can be applied to any cell harboring a mutation in the ARSA gene. The skilled worker will appreciate that cells that require active endogenous ARSA are of particular interest. Accordingly, in certain embodiments, the methods are applied to any cell that has lost endogenous ARSA activity. In certain embodiments, the method is applied to a neuron and/or a glial cell. In certain embodiments, of particular interest are neurons and/or glial cells that require active endogenous ARSA. In certain embodiments, the method is applied to cells of the central nervous system, and/or cells of the peripheral nervous system. In certain embodiments, of particular interest are cells of the central nervous system and/or of the peripheral nervous system that require active endogenous ARSA. In certain embodiments, of particular interest are cells in the forebrain, midbrain, hindbrain, spinal cord, and any combination thereof. In certain embodiments, of particular interest are cells of a central nervous system region selected from the group consisting of the spinal cord, the motor cortex, the sensory cortex, the thalamus, the hippocampus, the putamen, the cerebellum (e.g., the cerebellar nuclei), and any combination thereof. In certain embodiments, of particular interest are cells of the pons and medulla in the brain, ascending fasciculus of the spinal cord, and any combination thereof. In certain embodiments, of particular interest are cells of a central nervous system region selected from the group consisting of the spinal cord, the motor cortex, the sensory cortex, the thalamus, the hippocampus, the putamen, the cerebellum (e.g., the cerebellar nuclei), and any combination thereof, that require active endogenous ARSA. In certain embodiments, of particular interest are motor neurons and astrocytic profiles in the central nervous system (CNS), oligodendrocytes (ascending fibers) in the CNS, cellular populations of the cerebral cortex in the CNS, and sensory neurons of the peripheral nervous system (PNS). In certain embodiments, of particular interest are oligodendrocytes, such as those in the dorsal fasciculus of the spinal cord. In certain embodiments, of particular interest are glial profiles in the central nervous system, including but not limited to, astrocytes, oligodendrocytes, Schwann cells, and any combination thereof. In certain embodiments, of particular interest are motor neurons, astrocytes, oligodendrocytes, cells of the cerebral cortex in the central nervous system, sensory neurons of the peripheral nervous system, glial cells of the peripheral nervous system (e.g., Schwann cells), and any combination thereof.

The methods disclosed herein can be performed in vitro for research purposes or can be performed ex vivo or in vivo for therapeutic purposes.

In certain embodiments, the cell to be transduced is in a mammalian subject and the AAV is administered to the subject in an amount effective to transduce the cell in the subject. Accordingly, in certain embodiments, the instant disclosure provides a method for treating a subject having a disease or disorder associated with an ARSA gene mutation, the method generally comprising administering to the subject an effective amount of a rAAV as disclosed herein. The subject can be a human subject, a non-human primate subject (e.g., a cynomolgus), or a rodent subject (e.g., a mouse) with an ARSA mutation. Any disease or disorder associated with an ARSA gene mutation can be treated using the methods disclosed herein. Suitable diseases or disorders include, without limitation, metachromatic leukodystrophy.

In certain embodiments, the foregoing methods employ a rAAV comprising: (a) an AAV capsid protein comprising the amino acid sequence of amino acids 203-736 of SEQ ID NO: 16, and a transfer genome comprising 5′ to 3′ following genetic elements: a 5′ ITR element (e.g., the 5′ ITR of SEQ ID NO: 18), an enhancer element (e.g., the enhancer element of SEQ ID NO: 58), a promoter sequence (e.g., the promoter sequence of SEQ ID NO: 25), a chimeric intron sequence (e.g., the chimeric intron sequence of SEQ ID NO: 32), a silently altered human ARSA coding sequence (e.g., the hARSA coding sequence of SEQ ID NO: 14), an SV40 polyadenylation sequence (e.g., the SV40 polyadenylation sequence of SEQ ID NO: 42), and a 3′ ITR element (e.g., the 3′ ITR of SEQ ID NO: 19); (b) an AAV capsid protein comprising the amino acid sequence of amino acids 138-736 of SEQ ID NO: 16, and a transfer genome comprising 5′ to 3′ following genetic elements: a 5′ ITR element (e.g., the 5′ ITR of SEQ ID NO: 18), an enhancer element (e.g., the enhancer element of SEQ ID NO: 58), a promoter sequence (e.g., the promoter sequence of SEQ ID NO: 25), a chimeric intron sequence (e.g., the chimeric intron sequence of SEQ ID NO: 32), a silently altered human ARSA coding sequence (e.g., the hARSA coding sequence of SEQ ID NO: 14), an SV40 polyadenylation sequence (e.g., the SV40 polyadenylation sequence of SEQ ID NO: 42), and a 3′ ITR element (e.g., the 3′ ITR of SEQ ID NO: 19); and/or (c) an AAV capsid protein comprising the amino acid sequence of SEQ ID NO: 16, and a transfer genome comprising 5′ to 3′ following genetic elements: a 5′ ITR element (e.g., the 5′ ITR of SEQ ID NO: 18), an enhancer element (e.g., the enhancer element of SEQ ID NO: 58), a promoter sequence (e.g., the promoter sequence of SEQ ID NO: 25), a chimeric intron sequence (e.g., the chimeric intron sequence of SEQ ID NO: 32), a silently altered human ARSA coding sequence (e.g., the hARSA coding sequence of SEQ ID NO: 14), an SV40 polyadenylation sequence (e.g., the SV40 polyadenylation sequence of SEQ ID NO: 42), and a 3′ ITR element (e.g., the 3′ ITR of SEQ ID NO: 19).

In certain embodiments, the foregoing methods employ a rAAV comprising: (a) an AAV capsid protein comprising the amino acid sequence of amino acids 203-736 of SEQ ID NO: 16, and a transfer genome comprising the nucleotide sequence set forth in any one of SEQ ID NO: 41, 44, 46, 47, 48, 49, 65, 67, 68, 69, 75, or 76; (b) an AAV capsid protein comprising the amino acid sequence of amino acids 138-736 of SEQ ID NO: 16, and a transfer genome comprising the nucleotide sequence set forth in any one of SEQ ID NO: 41, 44, 46, 47, 48, 49, 65, 67, 68, 69, 75, or 76; and/or (c) an AAV capsid protein comprising the amino acid sequence of SEQ ID NO: 16, and a transfer genome comprising the nucleotide sequence set forth in any one of SEQ ID NO: 41, 44, 46, 47, 48, 49, 65, 67, 68, 69, 75, or 76.

The methods disclosed herein are particularly advantageous in that they are capable of expressing an ARSA protein in a cell with high efficiency both in vivo and in vitro. In certain embodiments, the expression level of the ARSA protein is at least 10%, 20%, 30%, 40%, 50%, 60%, 70%, 80%, 90%, or 100% of the expression level of the endogenous ARSA protein in a cell of the same type that does not have a mutation in the ARSA gene. In certain embodiments, the expression level of the ARSA protein is at least 1.1, 1.2, 1.3, 1.4, 1.5, 1.6, 1.7, 1.8, 1.9, 2, 3, 4, 5, 6, 7, 8, 9, or 10 fold higher than the expression level of the endogenous ARSA protein in a cell of the same type that does not have a mutation in the ARSA gene. Any methods of determining the expression level of the ARSA protein can be employed including, without limitation, ELISA, Western blotting, immunostaining, and mass spectrometry.

In certain embodiments, transduction of a cell with an AAV composition disclosed herein can be performed as provided herein or by any method of transduction known to one of ordinary skill in the art. In certain embodiments, the cell may be contacted with the AAV at a multiplicity of infection (MOI) of 50,000; 100,000; 150,000; 200,000; 250,000; 300,000; 350,000; 400,000; 450,000; or 500,000, or at any MOI that provides for optimal transduction of the cell.

An AAV composition disclosed herein can be administered to a subject by any appropriate route including, without limitation, intravenous, intrathecal, intraperitoneal, subcutaneous, intramuscular, intranasal, topical or intradermal routes. In certain embodiments, the composition is formulated for administration via intravenous injection or subcutaneous injection.

### IV. AAV PACKAGING SYSTEMS

In another aspect, the instant disclosure provides packaging systems for recombinant preparation of a recombinant adeno-associated virus (rAAV) disclosed herein. Such packaging systems generally comprise: first nucleotide encoding one or more AAV Rep proteins; a second nucleotide encoding a capsid protein of any of the AAVs as disclosed herein; and a third nucleotide sequence comprising any of the rAAV genomes as disclosed herein, wherein the packaging system is operative in a cell for enclosing the transfer genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.).

Any AAV Rep protein can be employed in the packaging systems disclosed herein. In certain embodiments of the packaging system, the Rep nucleotide sequence encodes an AAV2 Rep protein. Suitable AAV2 Rep proteins include, without limitation, Rep 78/68 or Rep 68/52. In certain embodiments of the packaging system, the nucleotide sequence encoding the AAV2 Rep protein comprises a nucleotide sequence that encodes a protein having a minimum percent sequence identity to the AAV2 Rep amino acid sequence of SEQ ID NO: 22, wherein the minimum percent sequence identity is at least 70% (e.g., at least 75%, at least 80%, at least 85%, at least 90%, at least 95%, at least 98%, at least 99%, or 100%) across the length of the amino acid sequence of the AAV2 Rep protein. In certain embodiments of the packaging system, the AAV2 Rep protein has the amino acid sequence set forth in SEQ ID NO: 22.

In certain embodiments of the packaging system, the packaging system further comprises a forth nucleotide sequence comprising one or more helper virus genes. In certain embodiments of the packaging system, the packaging system further comprises a third vector, e.g., a helper virus vector, comprising the forth nucleotide sequence comprising the one or more helper virus genes. The third vector may be an independent third vector, integral with the first vector, or integral with the second vector.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4 and VA. In certain embodiments of the packaging system, where the helper virus is HSV, the HSV genome comprises one or more of HSV genes selected from the group consisting of UL5/8/52, ICPO, ICP4, ICP22 and UL30/UL42.

In certain embodiments of the packaging system, the first, second, and/or third vector are contained within one or more plasmids). In certain embodiments, the first vector and the third vector are contained within a first plasmid. In certain embodiments the second vector and the third vector are contained within a second plasmid.

In certain embodiments of the packaging system, the first, second, and/or third vector are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g. with one or more recombinant helper viruses, such as a adenovirus, poxvirus (such as vaccinia virus), herpes virus (including HSV, cytomegalovirus, or baculovirus, containing a first, and a second, and optionally a third vector as described herein), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more capsid proteins as described herein, and with a transfer genome as described herein being delivered in the form of a plasmid or a recombinant helper virus).

Accordingly, the instant disclosure provides a packaging system for preparation of a recombinant AAV (rAAV), wherein the packaging system comprises a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a forth nucleotide sequence comprising one or more helper virus genes.

### V. EXAMPLES

The recombinant AAV vectors disclosed herein mediate highly efficient gene transfer in vitro and in vivo. The following examples demonstrate the efficient restoration of the expression of the ARSA gene (which is mutated in certain human diseases, such as metachromatic leukodystrophy) using an AAV-based vector as disclosed herein. These examples are offered by way of illustration, and not by way of limitation.

### Example 1: Human ARSA Transfer Vectors

This example provides human ARSA transfer vectors T-001, pHMI-5000, pHMI-5003, and pHMI-hARSA1-TC-002 for expression of human ARSA (hARSA) in a cell (e.g., a human cell or a mouse cell) to which the vector is transduced.

### a) T-001

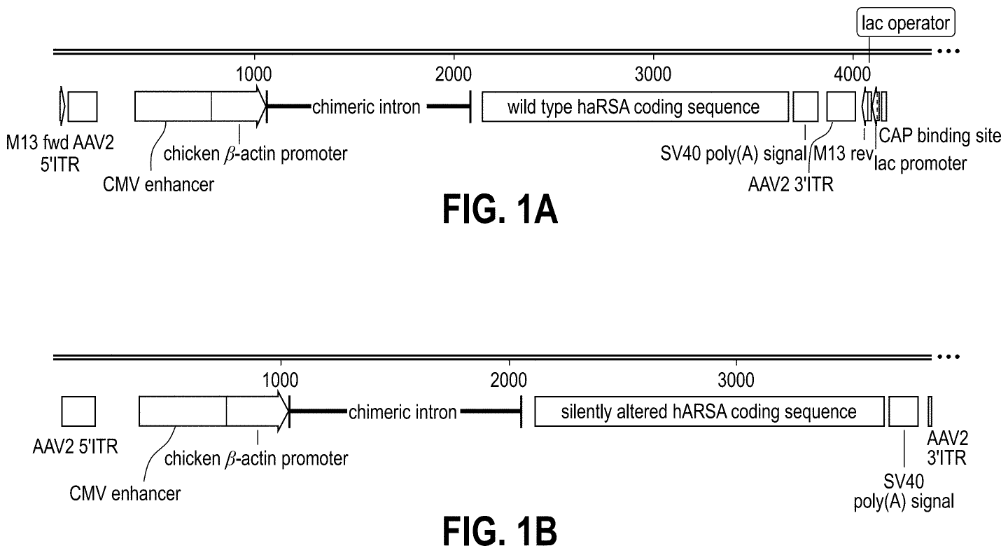

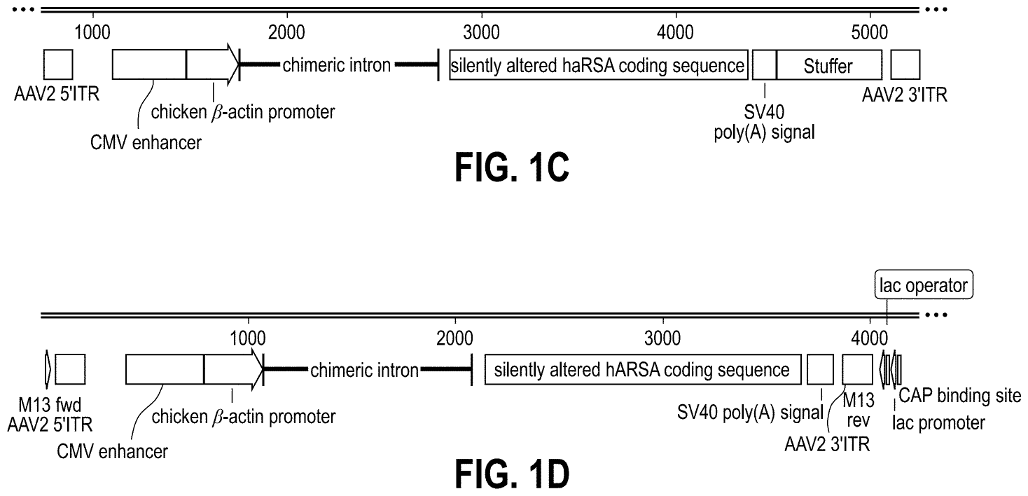

ARSA transfer vector TC-001, as shown in FIG. 1A, comprises 5′ to 3′ the following genetic elements: a 5′ ITR element, a transcriptional regulatory element comprising a CMV enhancer element, a chicken-β-actin promoter, and a chimeric intron sequence; a wild-type human ARSA coding sequence; an SV40 polyadenylation sequence; and a 3′ ITR element. The sequences of these elements are set forth in Table 1. This vector is capable of expressing a human ARSA protein in a cell (e.g., a human cell or a mouse cell) to which the vector is transduced.

b) pHMI-5000

ARSA transfer vector pHMI-5000, as shown in FIG. 1B, comprises 5′ to 3′ the following genetic elements: a 5′ ITR element; a transcriptional regulatory element comprising a CMV enhancer element, a chicken-β-actin promoter, and a chimeric intron sequence; a silently-altered human ARSA coding sequence; an SV40 polyadenylation sequence; and a 3′ ITR element. The sequences of these elements are set forth in Table 1. This vector is capable of expressing a human ARSA protein in a cell (e.g., a human cell or a mouse cell) to which the vector is transduced.

c) pHMI-5003

ARSA transfer vector pHMI-5003, as shown in FIG. 1C, comprises 5′ to 3′ the following genetic elements: a 5′ ITR element; a transcriptional regulatory element comprising a CMV enhancer element, a chicken-β-actin promoter, and a chimeric intron sequence; a silently-altered human ARSA coding sequence; an SV40 polyadenylation sequence; a non-coding stuffer sequence, and a 3′ ITR element. The sequences of these elements are set forth in Table 1. This vector is capable of expressing a human ARSA protein in a cell (e.g., a human cell or a mouse cell) to which the vector is transduced.

d) pHMI-hARSA1-TC-002

ARSA transfer vector pHMI-hARSA1-TC-002, as shown in FIG. 1D, comprises 5′ to 3′ the same genetic elements as pHMI-5000. The sequences of these elements are set forth in Table 1. The difference between pHMI-hARSA1-TC-002 and pHMI-5000 lies in the vector backbone sequence. This vector is capable of expressing a human ARSA protein in a cell (e.g., a human cell or a mouse cell) to which the vector is transduced.

TABLE 1
Genetic elements in human ARSA transfer vectors T-
001, pHMI-5000, pHMI-5003, and pHMI-hARSA1-TC-002

| | | | | pHMI- |
|---|---|---|---|---|
| | | | | hARSA1-TC- |
| Genetic | T-001 | pHMI-5000 | pHMI-5003 | 002 |
| Element | SEQ ID NO: | | | |
| 5′ ITR element | 18 | 18 | 18 | 18 |
| Enhancer | 58 | 58 | 58 | 58 |
| element | | | | |
| Promoter | 25 | 25 | 25 | 25 |
| sequence | | | | |
| Intron sequence | 32 | 32 | 32 | 32 |
| Transcriptional | 36 | 36 | 36 | 36 |
| regulatory | | | | |
| element | | | | |
| Human ARSA | 24 | 14 | 14 | 14 |
| coding sequence | | | | |
| SV40 | 42 | 42 | 42 | 42 |
| polyadenylation | | | | |
| sequence | | | | |
| Stuffer sequence | N/A | N/A | 39 | N/A |
| 3′ ITR element | 19 | 19 | 19 | 19 |
| Transfer genome | 41 | 44 | 46 | 44 |
| (from promoter | | | | |
| to | | | | |
| polyadenylation | | | | |
| sequence) | | | | |
| Transfer genome | 47 | 48 | 49 | 48 |
| (from 5′ ITR to | | | | |
| 3′ ITR) | | | | |
| Full vector | 50 | 51 | 52 | 53 |
| sequence | | | | |

The vectors disclosed herein can be packaged in an AAV capsid, such as, without limitation, an AAVHSC5, AAVHSC7, AAVHSC15 or AAVHSC17 capsid. The packaged viral particles can be administered to a wild-type animal, or an ARSA-deficient animal.

### Example 2: ARSA Gene Transfer in an ARSA(−/−) Mouse Model

In order to study the effect of ARSA gene transfer in mice, an ARSA(−/−) mouse model was generated. The ARSA(−/−) mouse model is an ARSA knock-out mouse produced by insertion of a neomycin cassette into exon 4 of the mouse ARSA gene (see, Hess et al., Proc. Natl. Acad. Sci. U.S.A. 1996, 93(25):14821-14826, incorporated by reference herein in its entirety). ARSA(−/−) mice develop similar but milder metachromatic leukodystrophy (MLD) compared to humans. ARSA(−/−) mice do not show evidence of widespread demyelination.

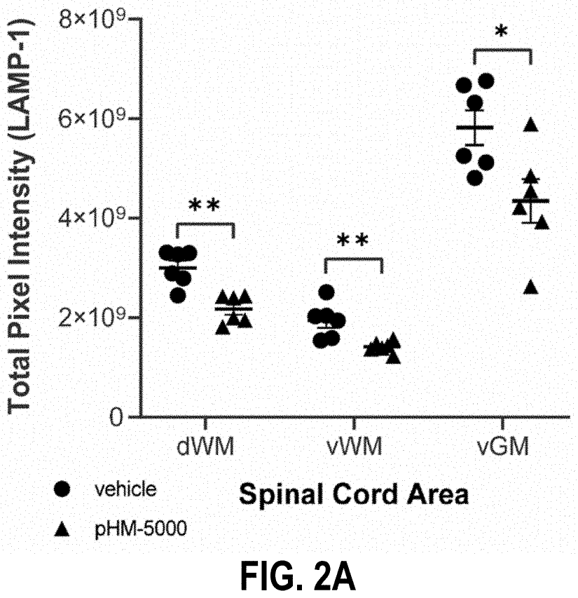

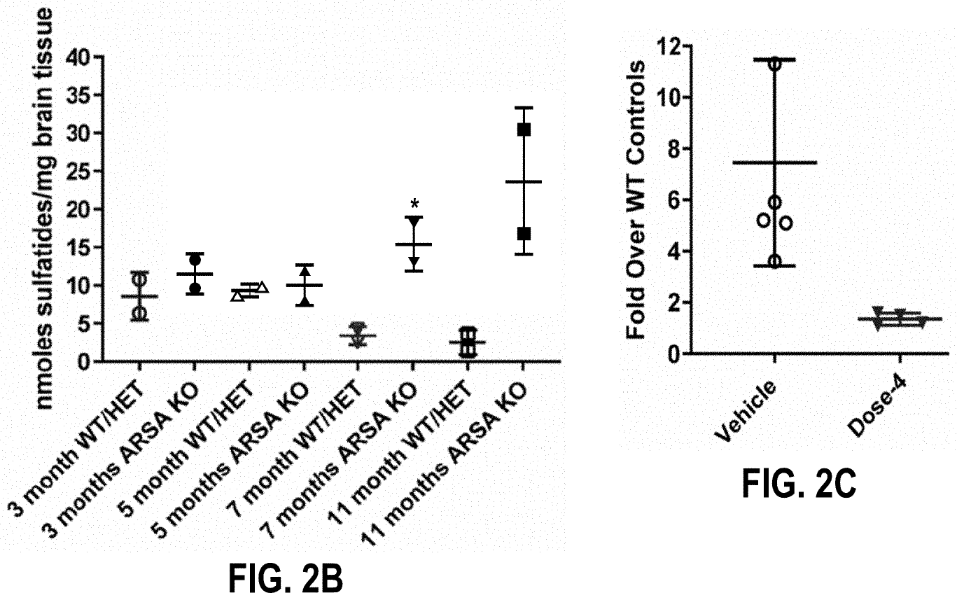

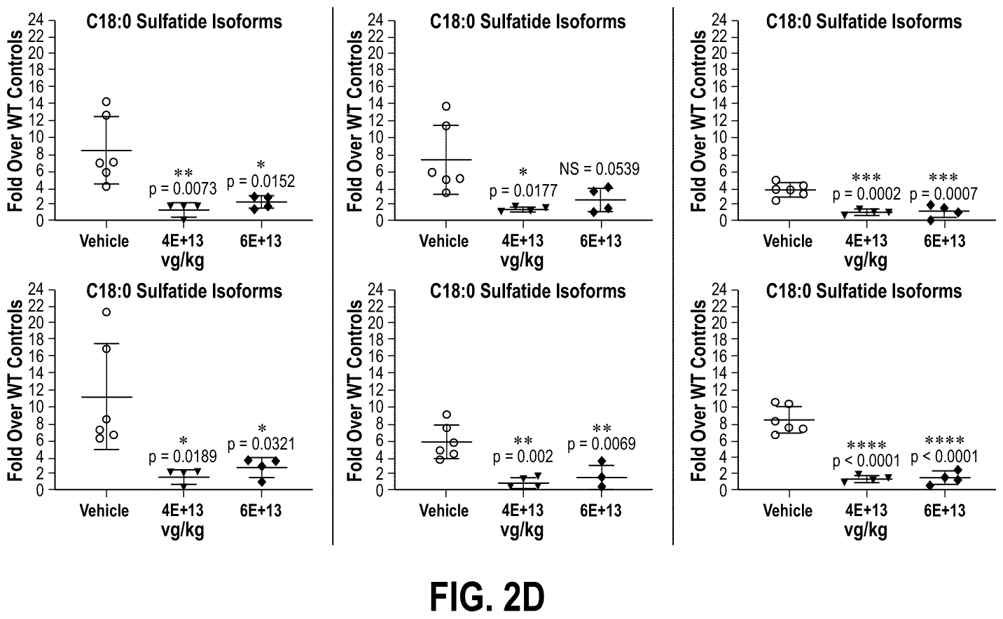

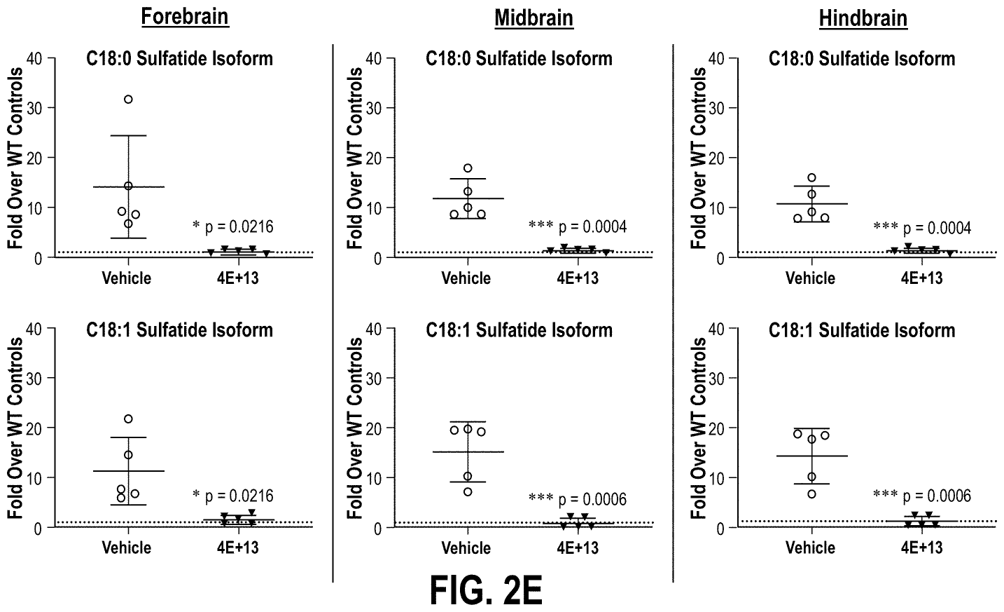

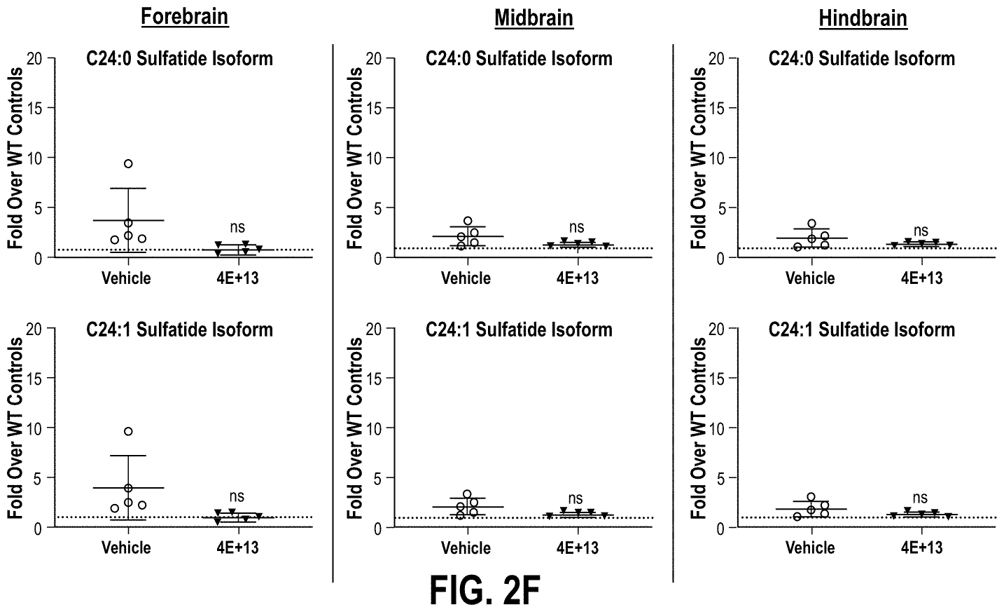

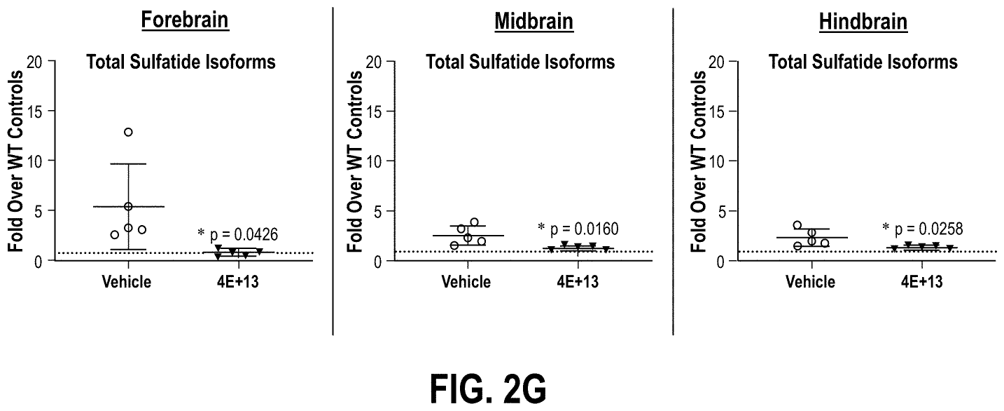

Various biomarkers can be used to investigate MLD. For example, the level of sulfatides in the brain can be measured. An increase in oligodendrocyte (C24:0) and neuronal (C18:0) sulfatide has been reported with accumulation increasing as the animal ages. The level of myelin and lymphocyte protein (MAL) mRNA transcript can be measured. MAL is expressed by oligodendrocytes and Schwann cells, stabilize glial-axon junctions, and has been implicated in the pathology of MLD. The level of MAL transcript has been reported to be reduced in ARSA(−/−) mice. Lysosomal-associated membrane protein (LAMP-1) is another biomarker that can be used to investigate MLD. LAMP-1 immunoreactivity has been investigated by immunohistochemistry on spinal cord tissue in ARSA(−/−) and wild type mice using an anti-LAMP-1 antibody, showing an increase in LAMP-1 immunoreactivity in ARSA(−/−) mice. FIG. 2A shows a quantification of total pixel intensity derived from LAMP-1 immunoreactivity investigated by immunohistochemistry (IHC) on spinal cord tissue from ARSA(−/−) mice. IHC was performed using an anti-LAMP-1 antibody in ARSA(−/−) mice treated with vehicle control or pHMI-5000 packaged in AAVHSC15 capsid. As shown in FIG. 2A, at 12 weeks post-dosing (4e13 vg/kg of pHMI-5000 packaged in AAVHSC15 capsid), a significant decrease in the level of LAMP-1 was detected compared to ARSA(−/−) animals dosed with vehicle control.

Brain tissue was weighed and homogenized in 250 uL of water in a Precellys bead homogenizer and a 10 uL aliquot of the homogenate was removed for Pierce BCA protein assay quantification. 760 uL of acetonitrile was added to each homogenate and the mixture was homogenized a second time. The homogenate was centrifuged at 14,000×g for 15 minutes and the centrifuge clarified supernatant was removed and diluted 5× in 75% acetonitrile for RapidFire-MS analysis. C19:0 sulfatide (Matreya cat #1888) was used as the internal standard and monitored together with C18:0, C18:1, C24:0 and C24:1 sulfatides in MRM mode on a Sciex API4000 triple quadrupole mass spectrometer. Each sample was injected 8 times with 8 different concentrations of C19:0 sulfatide IS to generate a unique standard curve for each sample which was used to calculate the concentration of each analyte. FIG. 2B shows the level of C18:0 sulfatides in the brains of control group mice (WT/Het) and ARSA(−/−) mice over time. The control group was a mix of wild type animals (ARSA(+/+)) and heterozygous animals (ARSA(+/−)). As shown in FIG. 2B, the level of C18:0 sulfatides in the brains of ARSA(−/−) mice accumulate over time, while the level of C18:0 sulfatides in the brains of control group mice largely remain unchanged over time. The data in FIG. 2B was generated from an analysis of two control group mice and two ARSA(−/−) mice. To investigate the effect of ARSA gene delivery on sulfatide accumulation in ARSA deficient mice, ARSA(−/−) mice were treated with 4e13 vg/kg of pHMI-hARSA1-TC-002 packaged in AAVHSC15 capsid (FIG. 2C). As shown in FIG. 2C, a significant decrease in brain sulfatide levels in treated ARSA(−/−) mice was observed at seven months post-dosing as compared to ARSA(−/−) mice treated with vehicle control.

C18:0 and C18:1 sulfatide isoform levels in the forebrain, midbrain, and hindbrain of ARSA(−/−) mice were determined seven months post-treatment with 4e13 vg/kg and 6e13 vg/kg of pHMI-5000 packaged in AAVHSC15 capsid, or a vehicle control (FIG. 2D). Sulfatide isoform levels are presented as fold over wild-type control animals of the same age. As shown in FIG. 2D, a significant decrease in brain sulfatide levels in all three brain regions of treated ARSA(−/−) mice was observed at seven months post-dosing as compared to ARSA(−/−) mice treated with a vehicle control. Methods and materials used were the same as above. Data was analyzed using an unpaired T-test.

C18:0 and C18:1 sulfatide isoform levels (FIG. 2E), C24:0 and C24:1 sulfatide isoform levels (FIG. 2F), and total sulfatide isoform levels (FIG. 2G) in the forebrain, midbrain, and hindbrain of ARSA(−/−) mice were determined 52 weeks post-treatment with 4e13 vg/kg of pHMI-5000 packaged in AAVHS15 capsid, or vehicle control. Methods and materials used were the same as above. Data was analyzed using an unpaired T-test.

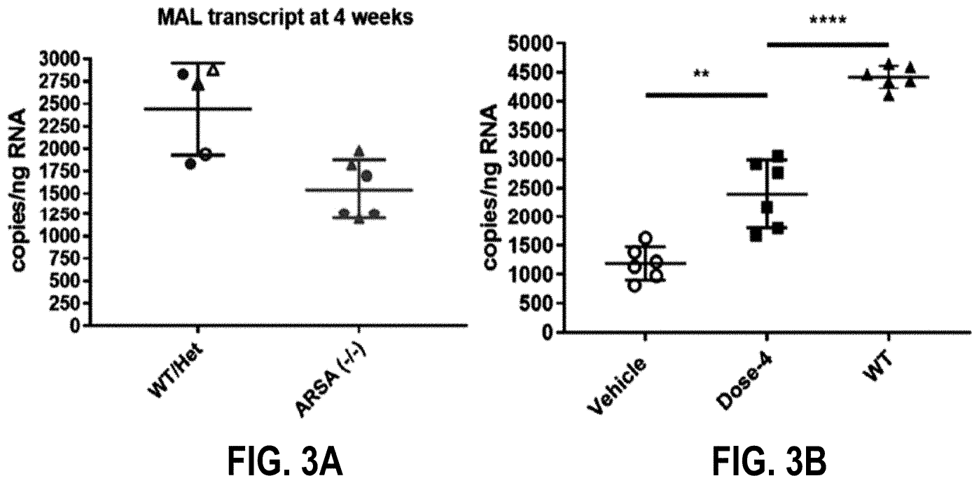

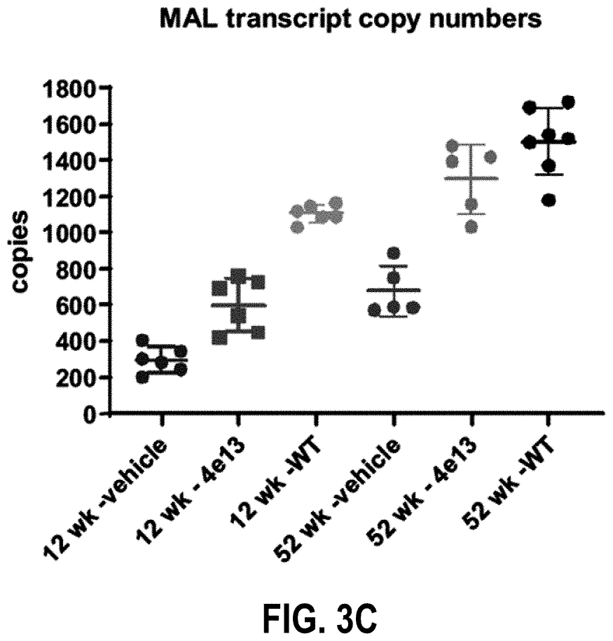

FIG. 3A shows the level of MAL transcript at four weeks in control group mice (WT/Het) and ARSA(−/−) mice. The control group was a mix of wild type animals (ARSA(+/+)) and heterozygous animals (ARSA(+/−)). Mouse total RNA was prepared with Trizol extraction followed by Qiagen RNEasy column purification. RNA was used as a template for cDNA synthesis using a ThermoFisher High Capacity cDNA Kit to produce transcript. MAL transcript was assessed using droplet digital PCR and primer/probe sets specific to mouse Myelin and Lymphocyte Protein (MAL) with copy number normalized to mouse HPRT1. As shown, at four weeks, the level of MAL transcript is decreased in the ARSA(−/−) mice compared to the heterozygous mice. The data in FIG. 3 was generated from an analysis of five control group mice and six ARSA(−/−) mice. To investigate the effect of ARSA gene delivery on the level of MAL transcript in ARSA deficient mice, ARSA(−/−) mice were treated with 4e13 vg/kg of pHMI-5000 packaged in AAVHSC15 capsid (FIG. 3B). As shown in FIG. 3B, a significant increase in MAL transcript levels in treated ARSA(−/−) mice was observed at three months post-dosing as compared to wild type mice and vehicle treated ARSA(−/−) mice.

The level of MAL transcript copy numbers in ARSA(−/−) mice treated with 4e13 vg/kg of pHMI-5000 packaged in AAVHSC15 capsid was determined (FIG. 3C). FIG. 3C shows the copy number of MAL transcript detected in wild type mice, or ARSA(−/−) mice administered vehicle control or 4e13 vg/kg of pHMI-5000 packaged in AAVHSC15 capsid, at 12 or 52 weeks post-dose. Methods and materials used were the same as above. Data was analyzed using an unpaired T-test. In FIG. 3C, statistical significance between animal groups are as follows: 12 week vehicle vs. treated animals, p=0.0012; 12 week treated vs wild type animals, p<0.0001; 52 week vehicle vs. treated animals, p=0.0004; and 52 week treated vs. wild type animals, not significant.

To investigate if therapeutic levels of hARSA activity can be achieved, transfer vector T-001 packaged in AAV9 capsid (see, PCT Publication No. WO2002/052052, incorporated by reference herein in its entirety) was administered into ARSA(−/−) mice. Anti-ARSA immunoreactivity of brain slices obtained from untreated control ARSA(−/−) mice, and ARSA(−/−) mice administered with transfer vector T-001 packaged in AAV9 capsid, show that hARSA enzyme activity at therapeutic levels (10%) was achieved at a dose of 2e13 vector genomes per kilogram body weight (vg/kg). Anti-ARSA immunoreactivity of brain slices obtained from treated ARSA(−/−) mice also show a dose dependent increase in ARSA enzyme activity in the brain.

### Example 3: ARSA Gene Transfer in an ARSA(−/−) Mouse Model

This example provides experimental data relating to the use of the human ARSA transfer vector pHMI-5000. As described herein, the transfer vector pHMI-5000 comprises a silently altered human ARSA coding sequence, which was shown to exhibit significantly improved expression of the ARSA protein.

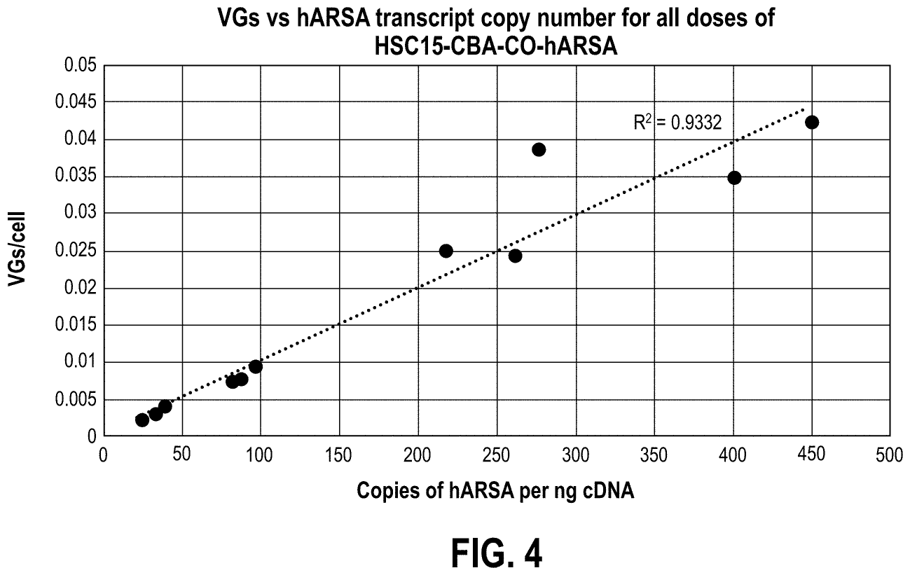

FIG. 4 is a plot showing that correlation between the number of vector genomes per transduced cell in the brain, and the number of copies of hARSA per ng of cDNA. Mouse genomic DNA was prepared using QIAamp Fast DNA Tissue Kit from Qiagen. VG counts were determined by droplet digital PCR and primer/probe sets specific to the coding region of the codon optimized human ARSA vector genome with normalization to endogenous mouse genomic sequence. Mouse total RNA was prepared as described herein and ARSA transcript was assessed using droplet digital PCR and the same primer/probe set used to determine VG counts with copy number normalized to mouse GUSB. As shown, for cells transduced using the transfer vector pHMI-5000 packaged in AAVHSC15 capsid, the number of vector genomes detected per transduced cell strongly correlates with the number of copies of hARSA per ng of cDNA (R²=0.9332).

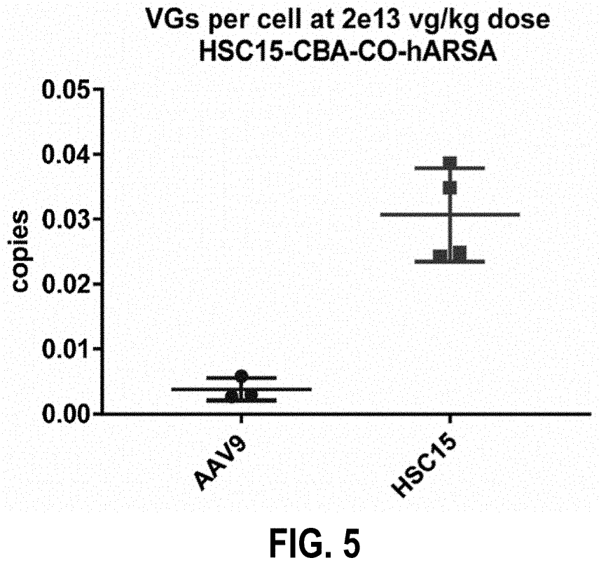

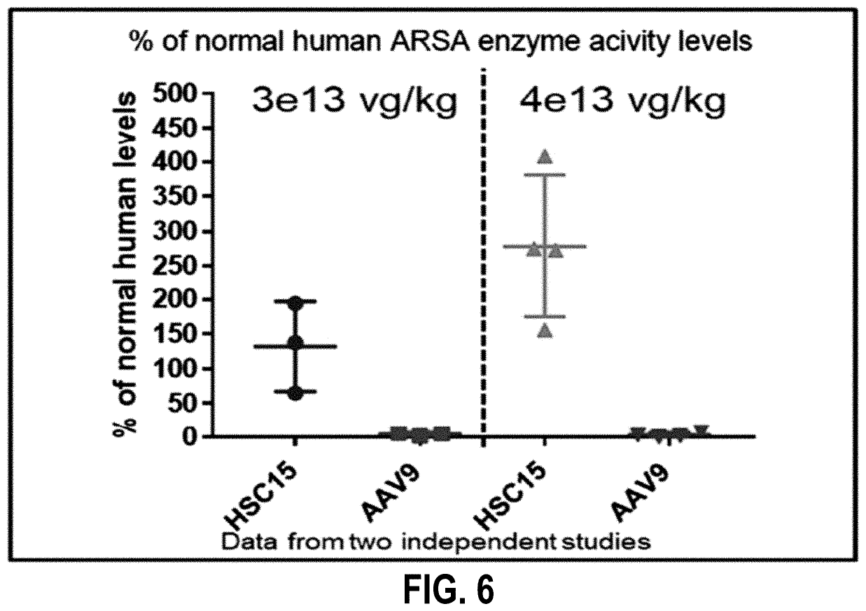

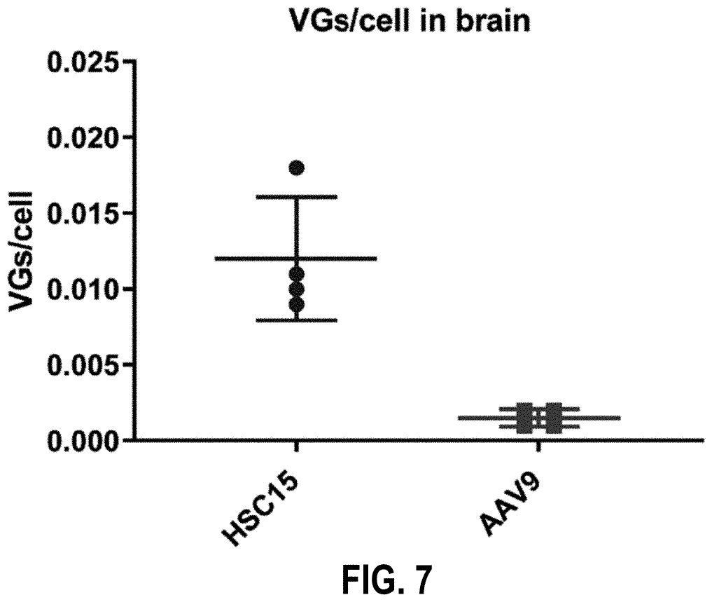

It was found that, in a comparison between AAVHSC15 and AAV9 capsid mediated delivery, AAVHSC15 significantly outperformed AAV9 in the brain. FIG. 5 shows the number of vector genomes per transduced cell in the brain at a dose of 2e13 vg/kg for transfer vector pHMI-5000 packaged in either AAV9 or AAVHSC15 capsid. As shown, ten-fold higher vector genome counts per cell were observed when the transfer vector pHMI-5000 was packaged in AAVHSC15 capsid, compared to AAV9 capsid. FIG. 6 shows the percent of normal human ARSA enzyme activity levels measured for transfer vector pHMI-5000 packaged in either AAV9 or AAVHSC15 capsid administered at the indicated doses. FIG. 7 shows the number of vector genomes per transduced brain cell in mice administered transfer vector pHMI-5000 packaged in either AAV9 or AAVHSC15 at 4e13 vg/kg.

pHMI-5000 packaged in AAVHSC15 capsid demonstrated a stronger and broader brain and spinal cord expression profile, compared to pHMI-5000 packaged in AAV9 capsid. Anti-ARSA immunoreactivity experiments show that much higher levels were detected in brain slices of mice intravenously administered pHMI-5000 packaged in AAVHSC15 capsid, compared to mice intravenously administered pHMI-5000 packaged in AAV9 capsid, in each case at a dose of 3e13 vg/kg.

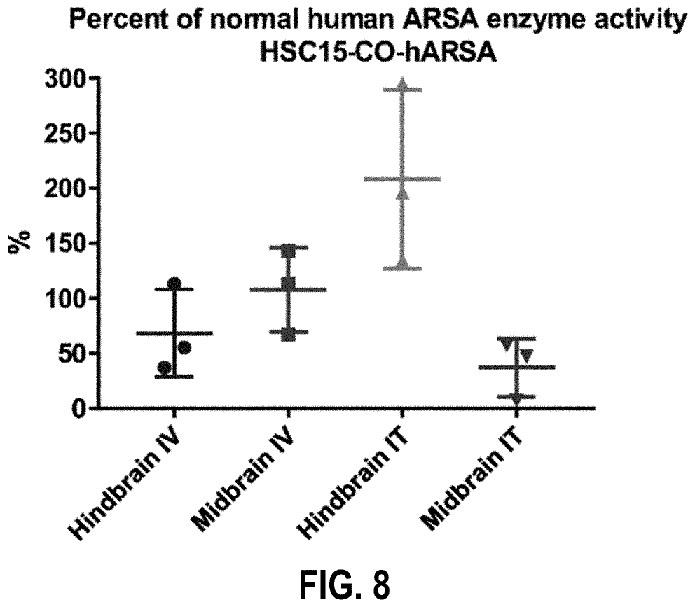

To evaluate the effect of route of administration on the biodistribution of hARSA in the brain, transfer vector pHMI-5000 packaged in AAVHSC15 capsid was administered through intravenous (IV) and intrathecal (IT) routes at a dose of 4e13 vg/kg and 4e12 vg/kg, respectively. Anti-ARSA immunoreactivity was present in key central nervous system regions following an IV dose of pHMI-5000 packaged in AAVHSC15 in ARSA(−/−) mice. Anti-mouse ARSA (mARSA) or human ARSA (hARSA) was detected broadly, including but not limited to motor and sensory cortex, hippocampus (CA3 region), putamen, and cerebellum. A quantification of percent of normal human ARSA enzyme activity in hindbrain and midbrain following IV or IT administration of transfer vector pHMI-5000 packaged in AAVHSC15 is shown in FIG. 8.

In ARSA(−/−) mice administered pHMI-5000 packaged in AAVHSC15 capsid at 4e13 vg/kg for 4 weeks, a biologically relevant distribution of hARSA was detected in key physiological regions of the brain as well as throughout the rostro-caudal axis of the central nervous system (CNS). hARSA was detected using an anti-hARSA antibody, and was detected in the spinal cord, motor cortex, thalamus, hippocampus, and cerebellar nucleus. hARSA was also detected in: motor neurons and astrocytic profiles in the CNS; oligodendrocytes in the CNS (with high detection in the ascending fibers); cellular populations of the cerebral cortex in the CNS; and sensory neurons and Schwann cells of the peripheral nervous system (PNS). A similar biological distribution can be detected as early as 2 weeks post-treatment.

In mice administered pHMI-5000 packaged in AAVHSC15 capsid at 2e13 vg/kg, the same histological distribution was observed as seen in mice administered a dose of at 4e13 vg/kg or higher. In these experiments, hARSA was detected in the cellular cytoplasm in a punctate pattern typical of that of lysosomes.

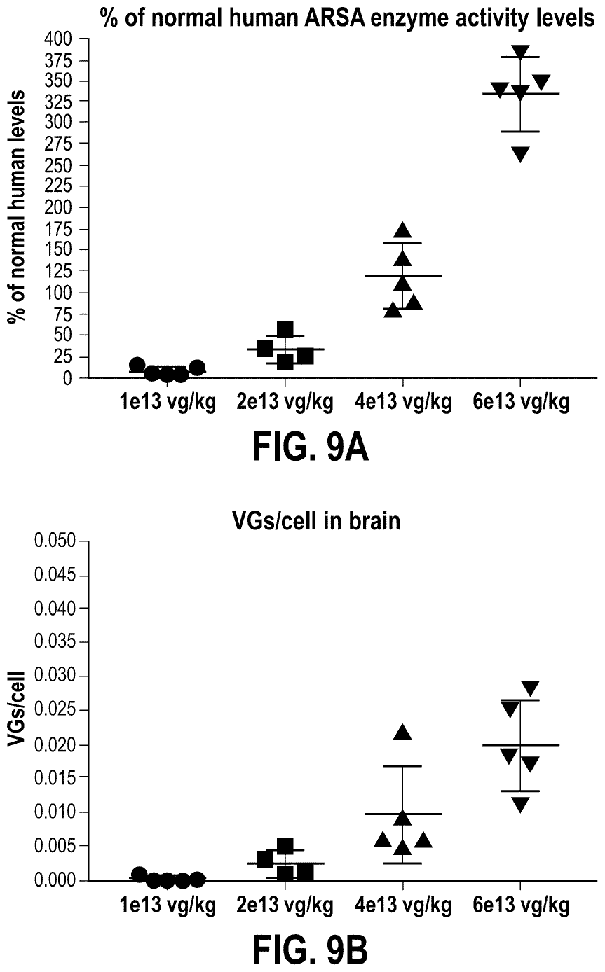

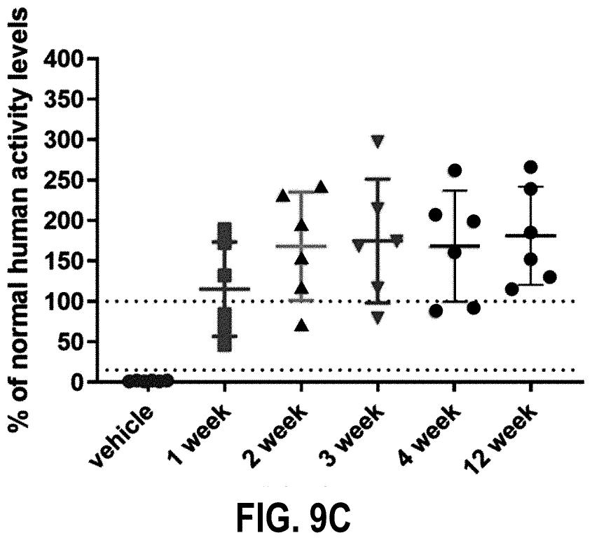

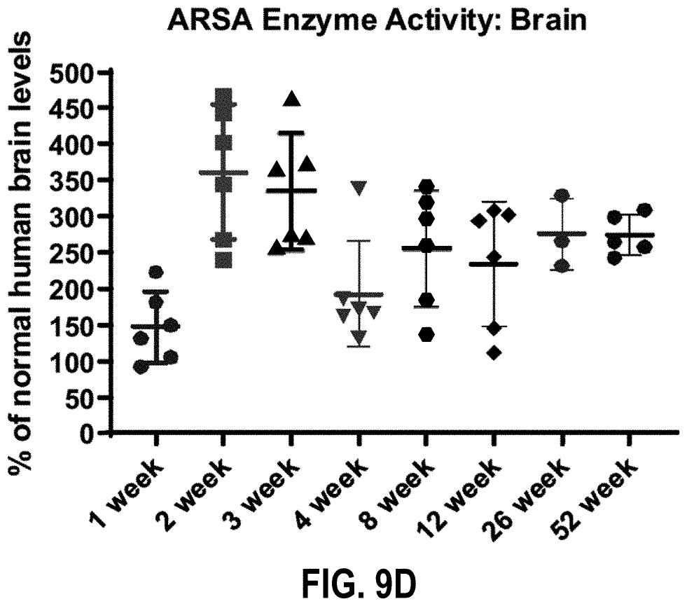

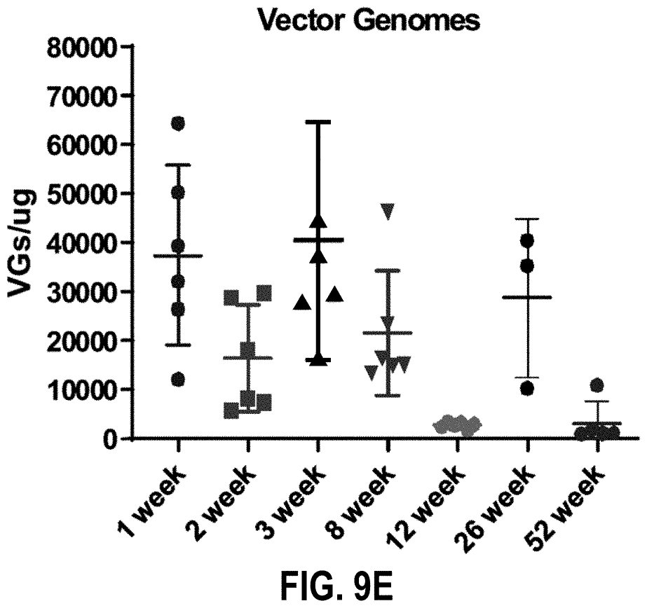

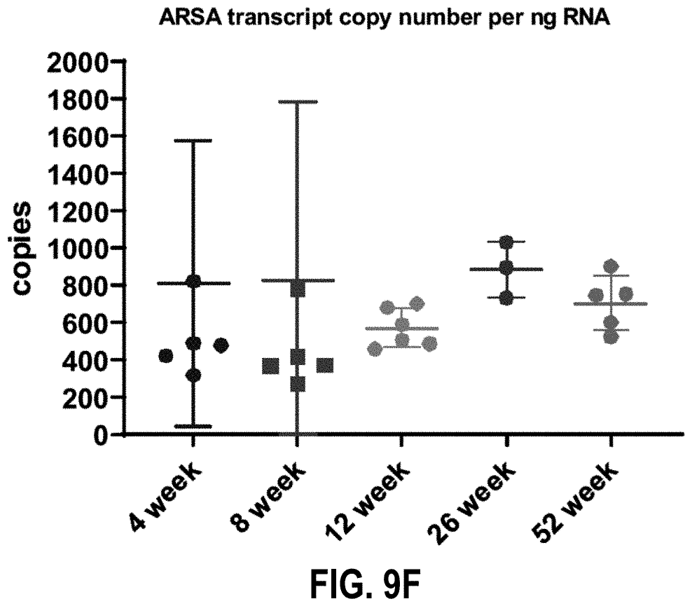

As shown in FIGS. 9A and 9B, the physiological level of human ARSA enzymatic activity was restored in the brains of treated ARSA(−/−) mice at 4 weeks post-dosing. Brain lysates from ARSA(−/−) mice were used for evaluating hARSA enzyme activity. A dose-range finding study showed that hARSA enzyme activity correlated with the dose of IV administration of transfer vector pHMI-5000 packaged in AAVHSC15 capsid. Enzymatic activity was detected in treated animals, but not in vehicle control animals. For the tested doses, the enzymatic activity levels (about 40-145%) were well above the therapeutic target of about 10-15%, as previously determined in the clinic (see, Patil and Maegawa, Drug Des. Devel. Ther. 2013, 7:729-745). FIG. 9A shows the percentage of normal hARSA activity achieved by administration of transfer vector pHMI-5000 packaged in AAVHSC15 capsid to ARSA(−/−) mice at the indicated doses. As shown, a dose-dependent response of hARSA activity was achieved. FIG. 9B shows the number of vector genomes per cell in brain of ARSA(−/−) mice administered transfer vector pHMI-5000 packaged in AAVHSC15 capsid at the indicated doses. For the 1e13 vg/kg, 4e13 vg/kg, and 6e13 vg/kg doses, n=5 mice. For the 2e13 vg/kg dose, n=4 mice. All mice were 5 weeks of age and all males. In FIG. 9C, ARSA enzymatic activity was assessed using a colorimetric Arylsulfatase A-specific assay that measures the cleavage of sulfate from the soluble substrate p-nitrocatechol-sulfate (pNCS). Non-specific cleavage of sulfate from competing enzymes is eliminated by use of an Arlysulfatase A-specific immunoprecipitation step. The normal human ARSA enzyme activity in brain is determined by analysis of ARSA enzyme activity in the frontal cortex of two each normal human males and females. Human frontal cortex samples were purchased from BioiVT and are run in triplicate alongside test samples on each ARSA enzyme activity assay plate. Data is expressed as a percent of the average amount of desulfated pNCS (in ng), per mg of protein per hour. FIG. 9C shows that a single intravenous 4e13 vg/kg dose of pHMI-5000 packaged in AAVHSC15 capsid resulted in the detection of hARSA enzyme activity in the brains of neonate ARSA(−/−) mice, as early as 1 week post-treatment and up to 12 weeks post-treatment, at levels exceeding the established human therapeutic target of 10-15% (as indicated with dashed line). Material was collected at 1, 2, 3, 4, and 12 weeks post-dose. n=6 mice for each timepoint, 3 males and 3 females at 8 weeks of age.

In FIG. 9D, mouse total RNA was prepared with Trizol extraction followed by Qiagen RNEasy column purification. RNA was used as a template for cDNA synthesis using ThermoFisher High Capacity cDNA Kit to produce transcript. ARSA transcript was assessed using droplet digital PCR and primer/probe sets specific to codon optimized human ARSA transcript, with copy number normalized to mouse GUSB. FIG. 9D shows that a single intravenous 4e13 vg/kg dose of pHMI-5000 packaged in AAVHSC15 capsid resulted in the detection of normal levels of hARSA enzyme activity (via hARSA transcript analysis) in the brains of adult ARSA(−/−) mice, as early as 1 week post-treatment. Peak levels of hARSA enzymatic activity were observed between 2 and 3 weeks post-dose, followed by a steady-state plateau sustained out to 52 weeks post-treatment, at levels exceeding the established human therapeutic target of 10-15%. Material was collected at 1, 2, 3, 4, 8, 12, 26, and 52 weeks post-dose. FIG. 9E shows the number of vector genomes per ug of genomic DNA in brains of ARSA(−/−) mice administered a single intravenous 4e13 vg/kg dose of pHMI-5000 packaged in AAVHSC15 capsid. Material was collected at 1, 2, 3, 8, 12, 26, and 52 weeks post-dose. FIG. 9F shows the number of copies of ARSA transcript per ng of RNA in brains of ARSA(−/−) mice administered a single intravenous 4e13 vg/kg dose of pHMI-5000 packaged in AAVHSC15 capsid. Material was collected at 4, 8, 12, 26, and 52 weeks post-dose.

### Example 4: Human ARSA Transfer Vectors

This example provides human ARSA transfer vectors TC-013.pHMIA2 and TC-015.pKITR for expression of hARSA in a cell (e.g., a human cell or a mouse cell) to which the vector is transduced. In addition to expressing hARSA, these vectors are designed to also express human SUMF1. The coding sequences of hARSA and hSUMF1 are separated by a 2A element. In certain embodiments, the ribosomal skipping element (e.g., 2A element) encodes a peptide that further comprises a sequence of Gly-Ser-Gly at the N terminus, optionally wherein the sequence of Gly-Ser-Gly is encoded by the nucleotide sequence of GGCAGCGGA. While not wishing to be bound by theory, it is hypothesized that ribosomal skipping elements function by: terminating translation of the first peptide chain and re-initiating translation of the second peptide chain; or by cleavage of a peptide bond in the peptide sequence encoded by the ribosomal skipping element by an intrinsic protease activity of the encoded peptide, or by another protease in the environment (e.g., cytosol).

a) TC-013.pHMIA2

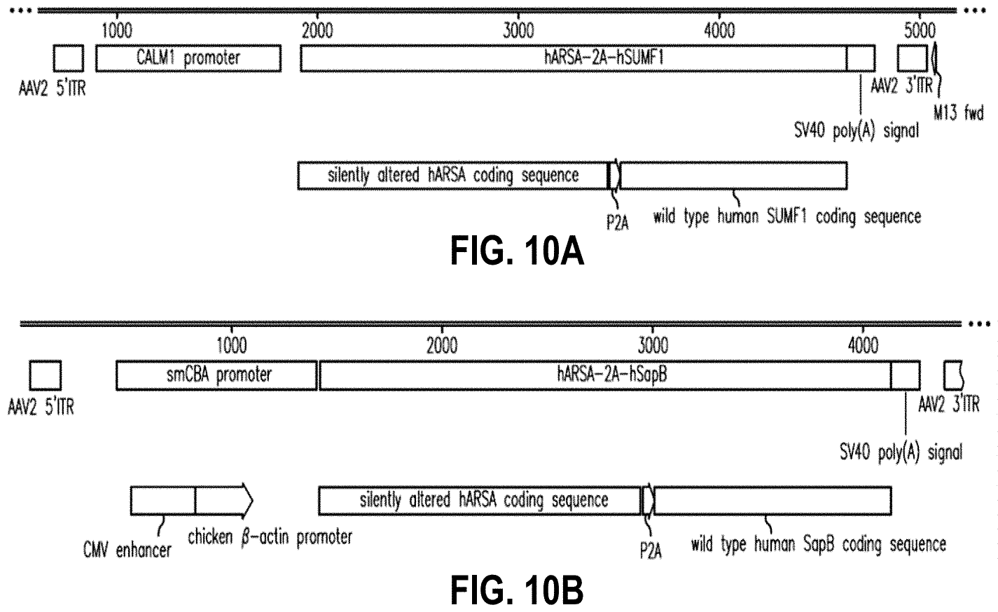

ARSA transfer vector TC-013.pHMIA2, as shown in FIG. 10A, comprises 5′ to 3′ the following genetic elements: a 5′ ITR element, a transcriptional regulatory element comprising a CALM1 promoter; a silently altered human ARSA coding sequence; a 2A element; a silently altered human SUMF1 coding sequence; and a 3′ ITR element. The sequences of these elements are set forth in Table 2. This vector is capable of expressing a human ARSA protein and a human SUMF1 protein in a cell (e.g., a human cell or a mouse cell) to which the vector is transduced.

b) TC-015.pKITR

ARSA transfer vector TC-015.pKITR, as shown in FIG. 10B, comprises 5′ to 3′ the following genetic elements: a 5′ ITR element, a transcriptional regulatory element comprising a smCBA promoter; a silently altered human ARSA coding sequence; a 2A element; a silently altered human SUMF1 coding sequence; and a 3′ ITR element. The sequences of these elements are set forth in Table 2. This vector is capable of expressing a human ARSA protein and a human SUMF1 protein in a cell (e.g., a human cell or a mouse cell) to which the vector is transduced.

TABLE 2
Genetic elements in human ARSA transfer
vectors TC-013.pHMIA2 and TC-015.pKITR

| | | |
|---|---|---|
| Genetic | TC-013.pHMIA2 | TC-015.pKITR |

| | |
|---|---|
| Element | SEQ ID NO: |

| | | |
|---|---|---|
| 5′ ITR element | 18 | 18 |
| Promoter sequence | 54 | 55 |
| Transcriptional regulatory | 54 | 55 |
| element | | |
| Human ARSA coding | 62 | 62 |
| sequence | | |
| 2A element | 63 | 63 |
| Human SUMF1 coding | 64 | 64 |
| sequence | | |
| hARSA-2A-hSUMF1 | 30 | 30 |
| sequence | | |
| 3′ ITR element | 19 | 19 |
| Transfer genome (from | 65 | 67 |
| promoter to SUMF1 coding | | |
| sequence) | | |
| Transfer genome (from 5′ | 68 | 69 |
| ITR to 3′ ITR) | | |
| Full vector sequence | 70 | 71 |

The vectors disclosed herein can be packaged in an AAV capsid, such as, without limitation, an AAVHSC5, AAVHSC7, AAVHSC15 or AAVHSC17 capsid. The packaged viral particles can be administered to a wild-type animal, or an ARSA-deficient animal.

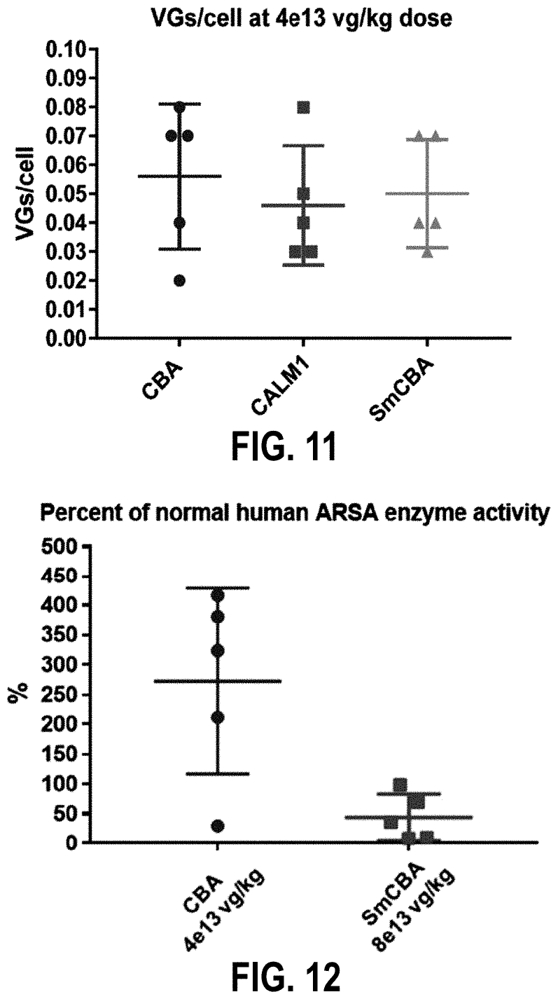

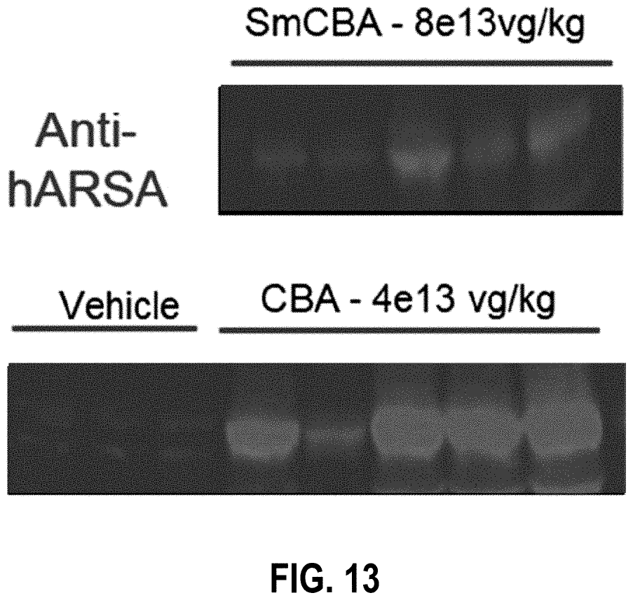

To evaluate the effect of promoters on hARSA expression in the brain, transfer vectors pHMI-5000, TC-013.pHMIA2, and TC-015.pKITR were packaged in AAVHSC15 capsid and administered to ARSA(−/−) mice intravenously. hARSA expression and enzyme activity was detected in brain with the pHMI-5000 vector (chicken-β-actin (CBA) promoter) administered at a dose of 4e13 vg/kg, and TC-015.pKITR (smCBA promoter) administered at a dose of 8e13 vg/kg, with similar viral genome per cell counts. The CBA promoter results in highest expression of hARSA at the lowest dose compared to other promoters tested. FIG. 11 shows the number of viral genomes transduced per cell for pHMI-5000 (CBA promoter), TC-013.pHMIA2 (CALM1 promoter), and TC-015.pKITR (smCBA promoter), in each case packaged in AAVHSC15 capsid and administered at a dose of 4e13 vg/kg (n=5 mice for each vector). FIG. 12 shows the percent of normal human ARSA enzyme activity detected for pHMI-5000 (CBA promoter) and TC-015.pKITR (smCBA promoter), in each case packaged in AAVHSC15 capsid and administered at a dose of 4e13 vg/kg (n=5 mice for each vector). FIG. 13 shows that expression of hARSA can be detected in brains of mice using an anti-hARSA antibody in Western blots for pHMI-5000 (CBA promoter) packaged in AAVHSC15 capsid and administered at a dose of 4e13 vg/kg, and TC-015.pKITR (smCBA promoter) packaged in AAVHSC15 capsid and administered at a dose of 8e13 vg/kg (n=5 mice for each vector).

### Example 5: Human ARSA Transfer Vectors

This example provides the human ARSA transfer vector pHMI-5004 for expression of hARSA in a cell (e.g., a human cell or a mouse cell) to which the vector is transduced. In addition to expressing hARSA, this vector is designed to also express human saposin B (SapB). The coding sequences of hARSA and SapB are separated by a 2A element.

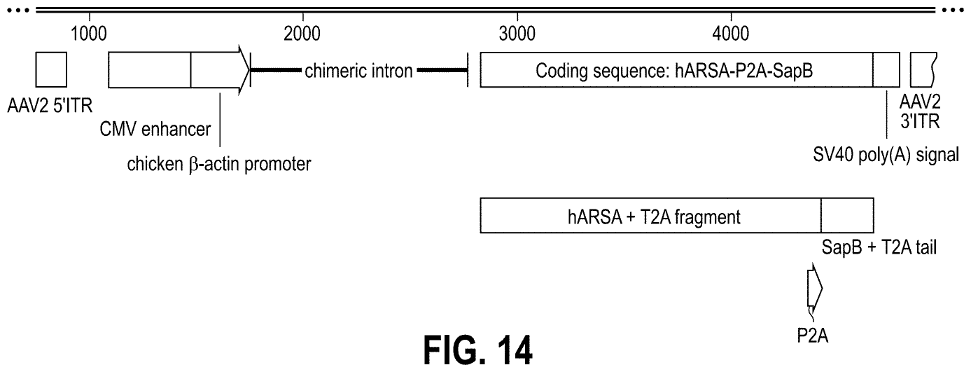

ARSA transfer vector pHMI-5004, as shown in FIG. 14, comprises 5′ to 3′ the following genetic elements: a 5′ ITR element; a transcriptional regulatory element comprising a CMV enhancer element, a chicken-β-actin promoter, and a chimeric intron sequence; a silently altered human ARSA coding sequence; a 2A element; a wild type human SapB coding sequence; and a 3′ ITR element. The sequences of these elements are set forth in Table 3. This vector is capable of expressing a human ARSA and/or SapB protein in a cell (e.g., a human cell or a mouse cell) to which the vector is transduced.

TABLE 3
Genetic elements in human ARSA transfer vector pHMI-5004

| Genetic Element | SEQ ID NO: |
|---|---|
| 5′ ITR element | 18 |
| Enhancer element | 58 |
| Promoter sequence | 25 |
| Intron sequence | 32 |
| Transcriptional regulatory element | 36 |
| Human ARSA coding sequence | 72 |
| 2A element | 63 |
| Human SapB coding sequence | 73 |
| hARSA-2A-hSapB sequence | 74 |
| SV40 polyadenylation sequence | 42 |
| 3′ ITR element | 19 |
| Transfer genome (from promoter to | 75 |
| polyadenylation sequence) | |
| Transfer genome (from 5′ ITR to 3′ ITR) | 76 |
| Full vector sequence | 77 |

### Example 6: ARSA Gene Transfer in Non-Human Primates

To investigate the effect of a single dose of AAVHSC-mediated ARSA gene delivery in non-human primates, six male naïve juvenile cynomolgus monkeys were dosed according to the experimental designs set forth in Tables 4 and 5.

TABLE 4
Experimental design for non-human primate studies

| | | Animals/ | Dose | | Volume | | Conc. | |
|---|---|---|---|---|---|---|---|---|
| Group | Route | Group | day | Dose (vg/kg) | (mL/kg) | | (vg/mL) | Necropsy |
| 1 | IV | 2 males | 1 | 0 | 5.0 | | — | Day |
| 2 | IV | 2 males | 1 | 4e13 | 5.0 | | 1.2e13 | 28/29 |
| 3 | CM | 2 males | 1 | Approx. 10% of | 0.5 | mL | Stock | |

| | |
|---|---|
| IV dose, given as | solution |
| fixed dose based | is 1.98 |
| on animal weight | vg/mL |
| (around 6e12 vg/kg) | |

TABLE 5
Experimental design for non-human primate studies

| | | | | Dose | |
|---|---|---|---|---|---|
| Animal | Weight (kg) | Treatment | Route | (vg/kg) | Vg/animal |
| 18C42 | 1.38 | Vehicle | IV | 0 | 0 |
| 18C17 | 1.55 | Vehicle | IV | 0 | 0 |
| 18C21 | 1.28 | AAVHSC15-pHMI-5005 | IV | 4e13 | 5.12e13 |
| 18C27 | 1.28 | AAVHSC15-pHMI-5005 | IV | 4e13 | 5.12e13 |
| 18C13 | 1.9 | AAVHSC15-pHMI-5005 | CM | 4e12 | 7.6e12 |
| 18C7 | 1.74 | AAVHSC15-pHMI-5005 | CM | 4e12 | 6.96e12 |

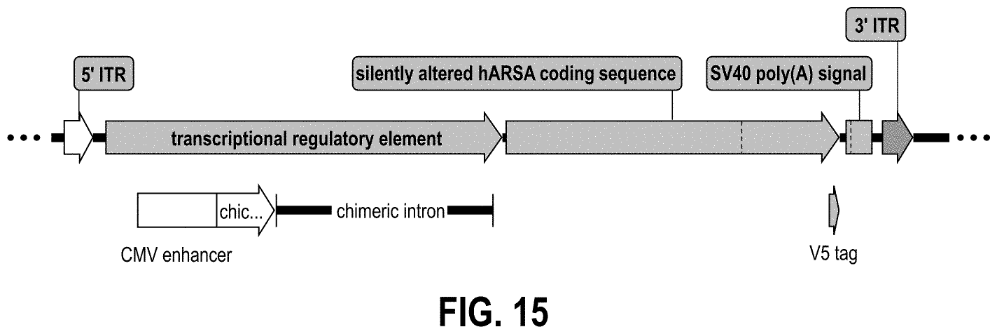

ARSA transfer vector pHMI-5005, as shown in FIG. 15, comprises 5′ to 3′ the following genetic elements: a 5′ ITR element; a transcriptional regulatory element comprising a CMV enhancer element, a chicken-β-actin promoter, and a chimeric intron sequence; a silently altered human ARSA coding sequence; a V5 tag; and a 3′ ITR element. The sequences of these elements are set forth in Table 6. This vector is capable of expressing a human ARSA protein in a cell (e.g., a human cell or a mouse cell) to which the vector is transduced.

TABLE 6
Genetic elements in human ARSA transfer vector pHMI-5005

| Genetic Element | SEQ ID NO: |
|---|---|
| 5′ ITR element | 18 |
| Enhancer element | 58 |
| Promoter sequence | 25 |
| Intron sequence | 32 |
| Transcriptional regulatory element | 36 |
| Human ARSA coding sequence | 14 |
| V5 tag | 78 |
| SV40 polyadenylation sequence | 42 |
| 3′ ITR element | 19 |
| Transfer genome (from promoter to | 79 |
| polyadenylation sequence) | |
| Transfer genome (from 5′ ITR to 3′ ITR) | 80 |
| Full vector sequence | 81 |

pHMI-5005 is a V5-tagged ARSA transfer vector. pHMI-5005 packaged in AAVHSC15 capsid was administered to non-human primates (NHP) according to the experimental design set forth in Tables 4 and 5. Administration was performed on Day 0 via 1-2 minute slow bolus intravenous injection (IV) via the cephalic/saphenous vein, or direct injection into the cisterna magna (CM). Viability checks were performed twice daily for signs of mortality and moribundity. Clinical observations were performed daily in the morning and on dose day after completion of the dose (15 min) and 4 hours post-dose. Blood for hematology and clinical chemistry was obtained immediately prior to dosing and at weeks 1, 2, and 4 post-dosing. At necropsy on days 28 and 29, following cerebrospinal fluid (CSF) and blood collections, animals were perfused with 1.0 L cold temperature saline to remove blood cells. Brain, liver, spinal cord (cervical and lumbar), cervical and lumbar dorsal root ganglion (DRG), trigeminal ganglia, kidney, sciatic nerve, peripheral lymph nodes, spleen, heart, lung, and testes were harvested at necropsy.

For bioanalytical analyses, serum is collected for V5 Elisa immediately prior to dosing, and at weeks 1, 2, and 4 (0.5 mL whole blood, processed to serum/split into two aliquots). 0.5 mL CSF was collected pre-dose (from Group 3 CM dosed animals) and 1-2 mL at necropsy (for all animals). 15 mL peripheral blood mononuclear cells (PBMC) were collected from whole blood prior to necropsy.

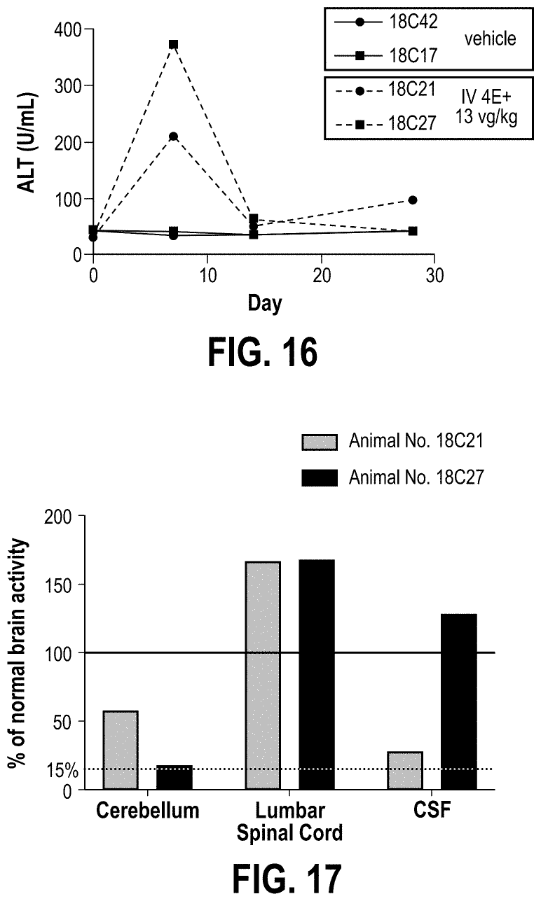

FIG. 16 shows an elevation in the level of alanine aminotransferase (ALT) in NHPs administered pHMI-5005 packaged in AAVHSC15 capsid. Elevated ALT returned to baseline levels by day 14 post-dosing.

NHPs that received a single IV dose of 4e13 vg/kg of pHMI-5005 packaged in AAVHSC15 (Group 2 animals) were sacrificed 28 and 29 days post-dosing. Human ARSA enzymatic activity levels were detected in the central nervous system (CNS) and cerebrospinal fluid (CSF) of sacrificed Group 2 animals (FIG. 17). As shown in FIG. 17, hARSA activity was detected at levels above the therapeutic threshold (15% of wild type human brain levels), as indicated by the dotted line. Immunofluorescence staining in the CNS and peripheral nervous system (PNS) of animal 18C27 (Group 2) confirms the presence of hARSA (via V5-tag detection), and in particular regions, including the dorsal root ganglion, spinal motor neurons, and cerebellum.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1.-40. (canceled)

41. A recombinant adeno-associated virus (rAAV) comprising:
(a) an AAV capsid comprising an AAV capsid protein; and
(b) a transfer genome comprising a transcriptional regulatory element operably linked to a silently altered ARSA coding sequence that comprises a nucleotide sequence set forth in SEQ ID NOs: 14, 62 or 72.

42. The rAAV of claim 41, wherein the silently altered ARSA coding sequence encodes an amino acid sequence set forth in SEQ ID NO: 23.

43.-44. (canceled)

45. The rAAV of claim 41, wherein the transcriptional regulatory element comprises:
a) one or more of the elements selected from the group consisting of a cytomegalovirus (CMV) enhancer element, a chicken-β-actin (CBA) promoter, a small chicken-β-actin (SmCBA) promoter, a calmodulin 1 (CALM1) promoter, a proteolipid protein 1 (PLP1) promoter, a glial fibrillary acidic protein (GFAP) promoter, a synapsin 2 (SYN2) promoter, a metallothionein 3 (MT3) promoter, and any combination thereof;
b) a nucleotide sequence at least 90% identical to a sequence selected from the group consisting of SEQ ID NO: 25, 32, 36, 54, 55, and 58;
c) a nucleotide sequence selected from the group consisting of SEQ ID NO: 25, 32, 36, 54, 55, and 58;
d) from 5′ to 3′ the nucleotide sequences set forth in SEQ ID NO: 58, 25, and 32; and/or
e) the nucleotide sequence set forth in SEQ ID NO: 36;
wherein the transfer genome optionally further comprises a polyadenylation sequence, optionally an exogenous polyadenylation sequence that:
is 3′ to the silently altered ARSA coding sequence, and optionally
comprises an SV40 polyadenylation sequence, optionally comprising the nucleotide sequence set forth in SEQ ID NO: 42.

46.-53. (canceled)

54. The rAAV of claim 41, wherein the transfer genome comprises a nucleotide sequence selected from the group consisting of SEQ ID NO: 41, 44, 46, 65, 67, 75, and 79.

55. The rAAV of claim 41, wherein the transfer genome comprises a 5′ inverted terminal repeat (5′ ITR) nucleotide sequence 5′ of the genome, and a 3′ inverted terminal repeat (3′ ITR) nucleotide sequence 3′ of the genome, optionally wherein the 5′ ITR nucleotide sequence has at least 95% sequence identity to SEQ ID NO: 18, and the 3′ ITR nucleotide sequence has at least 95% sequence identity to SEQ ID NO: 19.

56. (canceled)

57. The rAAV of claim 41, wherein the transfer genome comprises a nucleotide sequence selected from the group consisting of SEQ ID NO: 47, 48, 49, 68, 69, 76, and 80.

58. The rAAV of claim 41, wherein the nucleotide sequence of the transfer genome consists of a nucleotide sequence selected from the group consisting of SEQ ID NO: 47, 48, 49, 68, 69, 76, and 80.

59. (canceled)

60. The rAAV of claim 41, wherein the capsid protein comprises an amino acid sequence having at least 95% sequence identity with the amino acid sequence of amino acids 203-736 of SEQ ID NO: 2, 3, 4, 6, 7, 10, 11, 12, 13, 15, 16, or 17.

61. The rAAV of claim 60, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

62. The rAAV of claim 61, wherein:
(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R;
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C; or
(f) wherein the capsid protein comprises the amino acid sequence of amino acids 203-736 of SEQ ID NO: 2, 3, 4, 6, 7, 10, 11, 12, 13, 15, 16, or 17.

63. (canceled)

64. The rAAV of claim 41, wherein the capsid protein comprises an amino acid sequence having at least 95% sequence identity with the amino acid sequence of amino acids 138-736 of SEQ ID NO: 2, 3, 4, 5, 6, 7, 9, 10, 11, 12, 13, 15, 16, or 17, optionally wherein:
the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

65. (canceled)

66. The rAAV of claim 64, wherein:
(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 I(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is I or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

67. The rAAV of claim 41, wherein the capsid protein comprises:
a) the amino acid sequence of amino acids 138-736 of SEQ ID NO: 2, 3, 4, 5, 6, 7, 9, 10, 11, 12, 13, 15, 16, or 17; and/or
b) an amino acid sequence having at least 95% sequence identity with the amino acid sequence of amino acids 1-736 of SEQ ID NO: 2, 3, 4, 5, 6, 7, 8, 9, 10, 11, 12, 13, 15, 16, or 17; optionally wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

68.-69. (canceled)

70. The rAAV of claim 67, wherein:
(a) the amino acid in the capsid protein corresponding to amino acid 2 of SEQ ID NO: 16 is T, and the amino acid in the capsid protein corresponding to amino acid 312 of SEQ ID NO: 16 is Q;
(b) the amino acid in the capsid protein corresponding to amino acid 65 of SEQ ID NO: 16 is I, and the amino acid in the capsid protein corresponding to amino acid 626 of SEQ ID NO: 16 is Y;
(c) the amino acid in the capsid protein corresponding to amino acid 77 of SEQ ID NO: 16 is R, and the amino acid in the capsid protein corresponding to amino acid 690 of SEQ ID NO: 16 is K;
(d) the amino acid in the capsid protein corresponding to amino acid 119 of SEQ ID NO: 16 is L, and the amino acid in the capsid protein corresponding to amino acid 468 of SEQ ID NO: 16 is S;
(e) the amino acid in the capsid protein corresponding to amino acid 626 of SEQ ID NO: 16 is G, and the amino acid in the capsid protein corresponding to amino acid 718 of SEQ ID NO: 16 is G;
(f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(g) the amino acid in the capsid protein corresponding to amino acid 505 of SEQ ID NO: 16 is R, and the amino acid in the capsid protein corresponding to amino acid 687 of SEQ ID NO: 16 is R;
(h) the amino acid in the capsid protein corresponding to amino acid 346 of SEQ ID NO: 16 is A, and the amino acid in the capsid protein corresponding to amino acid 505 of SEQ ID NO: 16 is R;
(i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C; or
(j) the capsid protein comprises the amino acid sequence of amino acids 1-736 of SEQ ID NO: 2, 3, 4, 5, 6, 7, 8, 9, 10, 11, 12, 13, 15, 16, or 17.

71. (canceled)

72. A pharmaceutical composition comprising the rAAV of claim 41.

73. A polynucleotide comprising:
a) the nucleic acid sequence set forth in SEQ ID NO: 14, 62, or 72;
b) the nucleic acid sequence set forth in SEQ ID NO: 41, 44, 46, 65, 67, or 75; and/or
c) the nucleic acid sequence set forth in SEQ ID NO: 47, 48, 49, 68, 69, or 76.

74. A packaging system for preparation of an rAAV, wherein the packaging system comprises
(a) a first nucleotide sequence encoding one or more AAV Rep proteins;
(b) a second nucleotide sequence encoding a capsid protein of the AAV of claim 41; and
(c) a third nucleotide sequence comprising an rAAV genome sequence of the AAV of claim 41.

75.-79. (canceled)

80. A method for recombinant preparation of an rAAV, the method comprising introducing the packaging system of claim 74 into a cell under conditions whereby the rAAV is produced.

81.-83. (canceled)

84. A method for expressing an arylsulfatase A (ARSA) polypeptide in a cell, the method comprising transducing the cell with the recombinant adeno-associated virus (rAAV) of claim 41.

85. A method for treating a subject having metachromatic leukodystrophy (MLD), the method comprising administering to the subject an effective amount of the rAAV of claim 41.
